(19) United States
(12) Patent Application Publication
(10) Pub. No.: US 2026/0209287 A1
(43) Pub. Date: Jul. 23, 2026

(54) RECOMBINANT POLYPEPTIDE

(71) Applicant: HUMBLE BEE LIMITED, Dalmore, Dunedin (NZ)

(72) Inventors: Veronica HARWOOD-STEVENSON, Dunedin (NZ); Andrew WALKER, Dunedin (NZ); Thomas Walter Rizvi HARROP, Dunedin (NZ); Ryan GRAVES, Dunedin (NZ); James DICKSON, Dunedin (NZ)

(21) Appl. No.: 19/134,823

(22) PCT Filed: Dec. 1, 2023
(86) PCT No.: PCT/IB2023/062105

§ 371 Date: Jun. 2, 2025

(30) Foreign Application Priority Data
Dec. 2, 2022 (AU) 2022903684

(51) Int. Cl. C07K 14/435; A61L 27/34; C09D 189/00; D01F 4/02; D01F 6/90; D06M 15/15; D06N 3/12

(52) U.S. Cl. CPC C07K 14/43586; A61L 27/34; C09D 189/00; D01F 4/02; D01F 6/90; D06M 15/15; D06N 3/126; D10B 2401/063; D10B 2505/04; D10B 2509/00

(57) ABSTRACT

The invention relates generally to an isolated polypeptide from the solitary bee, Hylaeus nubilosus, a proteinaceous polymer comprising the polypeptide or a portion thereof, a composition comprising the polypeptide and/or polymer or portion thereof of either, and a method of recombinant production of the polypeptide and/or polymer or portion thereof.

### CROSS REFERENCE TO RELATED APPLICATIONS

This application is a 35 U.S.C. § 371 filing of International Patent Application No. PCT/IB2023/062105, filed Dec. 1, 2023, which claims priority to Australian Patent Application No. 2022903684, filed Dec. 2, 2022, the contents of each of which are incorporated herein by reference in their entirety.

### SEQUENCE LISTING

This application contains a sequence listing, which is submitted electronically via The United States Patent and Trademark Center Patent Center as an XML formatted sequence listing with a file name of “765285_BPIT-001US_ST26.xml” and a creation date of Jun. 2, 2025 and having a size of 101,180 bytes. The sequence listing submitted via Patent Center is part of the specification and is herein incorporated by reference.

### FIELD OF THE INVENTION

This invention generally relates to a polypeptide from the Colletid bee, Hylaeus nubilosus. The invention also relates to recombinant production of the polypeptide, to methods of making the polypeptide and to the use of the polypeptide to make various articles of manufacture comprising desirable properties.

### BACKGROUND

Synthetic polymers (components of “plastics”) are an exceptionally useful group of materials. Synthetic polymers are contained within many of the everyday products we dress in, sleep on, and create with. In many instances, synthetic polymers are used in the manufacture of various materials to impart desirable properties. Examples of such properties include imparting mechanical strength, heat resistance, wicking or the ability to repel water or resist wetting,

Industries where the use of synthetic polymers is prevalent include textiles, biomedical devices, and cosmetics. In one example, mass produced synthetic clothing is composed of various polymers that have been spun into synthetic fibers including polyester, nylon, vinyl, and acrylics. In some cases, these synthetic fibers are hydrophobic and are poor absorbents of water or sweat, impacting their wear comfort. In others, the fibers are hydrophilic and overly absorbent, again impacting wearability.

In other examples, synthetic polymers may be used to impart wicking, hygroscopic or hydrophilic properties to a textile or other material. Synthetic polymers currently being used for such applications are not eco-friendly. For example, polyether amines are used to form a hydrophilic coating on nylon clothing. Unfortunately, these polymers degrade over time, releasing harmful by-products that are toxic to aquatic life into the environment.

Environmental concerns associated with textile finishing chemicals have shifted the focus of major manufacturing companies toward green (bio-based) chemicals, which are eco-friendly. Green chemicals are produced using animal and plant fats/oils, making them eco-friendly and cost-efficient compared to their conventional counterparts. However, the additional weight of oil-based products, the need to reapply them, and the fluctuating availability and prices of raw material pose a challenge for the market players to achieve profitability and economies of scale.

Another important use of such coatings is in the medical device industry. Examples of various biomedical devices currently coated with hygroscopic/hydrophilic coatings include catheters, implants, tubes, lenses, and disposable plastic slides. In many examples, these coatings provide the coated biomedical devices (particularly those used in situ) with excellent biocompatibility, hydrophilicity, hydrophobicity and/or friction resistance, allowing for effective performance.

Typically, these coatings are composed of polyurethane, silicone, and polyethylene terephthalate materials,

Synthetic polymers are also used in the production of cosmetics/personal care products to impart properties like lubricity and viscosity. Phthalates are a group of chemicals called “everywhere” chemicals, found in products like nail polish, perfumes, deodorants, hair gels, shampoos, soaps, hair sprays, and body lotions. Phthalates have been identified as endocrine-disrupting chemicals in humans, causing hormonal imbalances, and numerous reproductive health, and developmental problems. Phthalates also bioaccumulate in fish, proving toxic to aquatic ecosystems and introducing hazards to humans,

The increasing public awareness of the environmental concerns associated with synthetic polymer use has led to an urgent need in the art for industries to provide alternatives that are non-toxic and eco-friendly.

It is an object of the present invention to provide a naturally occurring polypeptide or derivative thereof that is that may be used as an eco-friendly alternative for at least some of the synthetic polymers currently used in the various industries described above and/or to provide a method of making such a polypeptide or derivative thereof and/or to at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

Disclosed herein is a Hylaeus nubilosus polypeptide, also termed a “FUN” polypeptide. Also disclosed is a proteinaceous polymer comprising a H. nubilosus “FUN” polypeptide, or at least a portion of a Hylaeus nubilosus polypeptide. In some embodiments the proteinaceous polymer comprises a quasi-repeat domain. In some embodiments, the polymer is capable of assembly into a fiber. Also disclosed are compositions of such polypeptides and proteinaceous polymers and methods of producing and using said polypeptides, proteinaceous polymers and compositions.

Accordingly, in one aspect the present invention relates to an isolated polynucleotide encoding a polypeptide comprising at least 70% amino acid sequence identity to SEQ ID NO: 2.

In another aspect the invention relates to an isolated polynucleotide encoding a polypeptide comprising SEQ ID NO: 2.

In another aspect the invention relates to an isolated polypeptide comprising at least 70% amino acid sequence identity to SEQ ID NO: 2.

In another aspect the present invention relates to an isolated polynucleotide encoding a polypeptide comprising at least 70% amino acid sequence identity to

(SEQ ID NO: 6)
SESAVQSSKSSSGMSSQAQSQQQQX₁QLQQSQAQSQEQESQSAX₂.

In another aspect the invention relates to an isolated polynucleotide encoding a

polypeptide comprising
(SEQ ID NO: 6)
SESAVQSSKSSSGMSSQAQSQQQQX₁QLQQSQAQSQEQESQSAX₂.

In another aspect the invention relates to an isolated polypeptide comprising at least 70% amino acid sequence identity to

(SEQ ID NO: 6)
SESAVQSSKSSSGMSSQAQSQQQQX₁QLQQSQAQSQEQESQSAX₂.

In another aspect the invention relates to a vector that encodes an isolated polypeptide as described herein.

In another aspect the invention relates to a vector comprising an isolated polynucleotide as described herein.

In another aspect the invention relates to an isolated host cell comprising an isolated polypeptide, isolated polynucleotide, and/or vector as described herein.

In another aspect the invention relates to a proteinaceous polymer comprising a FUN polypeptide or portion thereof as described herein, wherein the proteinaceous polymer comprises at least one quasi-repeat domain.

In another aspect the invention relates to a composition comprising an isolated polypeptide, isolated polynucleotide, vector and/or proteinaceous polymer as described herein, and a carrier, diluent or excipient.

In another aspect the invention relates to a method of making an isolated FUN polypeptide or portion thereof selected from the group consisting of SEQ ID NO: 2, 4, 6, 8, 10, 12, 14, 16, 18, 20, 22, 24, 50 and 52, the method comprising heterologously expressing the FUN polypeptide in an isolated host cell, and optionally purifying the FUN polypeptide,

In another aspect the invention relates to a method of making a proteinaceous polymer comprising a FUN polypeptide or portion thereof selected from the group consisting of SEQ ID NO: 2, 4, 6, 8, 10, 12, 14, 16, 18, 20, 22, 24, 50 and 52, the method comprising heterologously expressing the FUN polypeptide or portion thereof in an isolated host cell under conditions that result in the expression of the FUN polypeptide or portion thereof, and optionally purifying the proteinaceous polymer.

In another aspect the invention relates to a polypeptide as described herein made by a method as described herein.

Various embodiments of the different aspects of the invention as discussed above are also set out below in the detailed description of the invention, but the invention is not limited thereto.

Other aspects of the invention may become apparent from the following description which is given by way of example only and with reference to the accompanying drawings.

### BRIEF DESCRIPTION OF THE DRAWINGS

The invention will now be described by way of example only and with reference to the drawings in which:

FIG. 1: ClustalW alignment of glutamine-rich sequences TRINITY_DN0_c0_g1_118 (SEQ ID NO: 34), TRINITY_DN0_c0_g1_113 (SEQ ID NO: 33), TRINITY_DN0_c0_g1_119 (SEQ ID NO: 35), TRINITY_DN0_c0_g1_120 (SEQ ID NO: 36), TRINITY_DN220917_c0_g1_11 (SEQ ID NO: 37), TRINITY_DN0_c0_g1_19 (SEQ ID NO: 38), TRINITY_DN0_c0_g1_11 (SEQ ID NO: 39), TRINITY_DN0_c0_g1_12 (SEQ ID NO: 40), TRINITY_DN0_c0_g1_116 (SEQ ID NO: 41), TRINITY_DN0_c0_g1_111_a (SEQ ID NO: 42), TRINITY_DN0_c0_g1_i11_b (SEQ ID NO: 43), TRINITY_DN0_c0_g1_110 (SEQ ID NO: 44), TRINITY_DN16733_c0_g1_12 (SEQ ID NO: 45) and TRINITY_DN0_c4_g1_12 (SEQ ID NO: 46) that were identified as significant from different entries in the transcript database (version 2).

FIG. 2: Consensus sequence alignment. Alignment of the FUN_069765. 2 sequence (from the long read sequence assembly), SEQ ID NO: 2, with the assembled proteomic sequences from the nest material mass spectrometry analysis, SEQ ID NO: 33, Consensus sequence: X=mismatches, Z=glutamic acid or glutamine.

FIG. 3: Summary flowchart of the Nest Material Gene identification. Data and samples are indicated by the unbroken box borders. Analysis and software programs are in bold in dashed boxes. Findings are in the boxes outlined with dots. Arrows imply a directional relationship between the boxes. Dashed arrows highlight the relationship between the findings.

FIG. 4: SDS-PAGE showing purified FUN polypeptide. The last three lanes exhibit IMAC elution of FUN polypeptide bands at an apparent molecular weight of ~100 kDa on the SDS-PAGE with a purity estimate of >95%.

FIG. 5: FTIR FUN polypeptide coating on foil. FTIR of FUN polypeptide & silk fibroin coated on aluminium foil with and without ethanol treatment.

FIG. 6: FTIR-FUN polypeptide coating on glass FTIR of FUN polypeptide coated with and without ethanol treatment on glass substrate.

FIG. 7. Scanning electron microscopy (SEM) images of FUN polypeptide coating. The image on the right-hand side is a magnified cross section of the first image depicting a thread-like structure along the film plane.

FIG. 8: Wettability—FUN polypeptide water contact angle profiles of FUN polypeptide FUN polypeptide upon contact and after 30 seconds. Comparison of water contact angle profile of FUN polypeptide and silk fibroin coated on glass substrate with and without ethanol treatment,

FIG. 9: Washability—The washability of FUN polypeptide coating and its ability to withstand water and PBS was assessed. The FUN polypeptide coating remained intact (shown by the coverage of Coomassie stain) after being soaked by water or PBS solutions for 24 hours.

FIG. 10: Sequence coverage with Glu-C digestion: Bold texts represent high confidence peptide match, normal text indicates moderate confidence, italic indicates poor confidence and underlined represents unobserved region.

FIG. 11: Combined sequence coverage with Glu-C and Trypsin digestions: Reduced and alkylated Trypsin digest yielded coverage at the C-terminal region with two high confidence peptides (bold) and one moderate peptide match (normal), giving an overall coverage of 95.78%.

FIG. 12: Alkylated Trypsin digest yielded an overall coverage of 99.81%,

Alkylated trypsin digestion picked up the entire stretch except the last cysteine residue, giving an overall coverage of 99.81%.

FIG. 13: Photographs of tubes and confocal images of A) oil/buffer emulsion (control); B) oil/HnM1M7-01 in buffer emulsion after days 1 and 2.

FIG. 14: Photographs of tubes and confocal images of A) oil/buffer emulsion (control) with no spheres observed in the water phase; B) oil/HnM1M7-01 in buffer emulsion showing stable spheres on day 7, reflecting the emulsion stabilising effect of HnM1M7-01.

FIG. 15: Water contact angles (measured in degrees) of glass slides with and without HnM1M7-03 coating,

FIG. 16: Water contact angles (measured in degrees) of glass slides with and without HnM7-06 coating.

FIG. 17: SEM surface morphology of wet spun nylon and HnP1-nylon (1, 5, and 10 wt. %) fibers.

FIG. 18: SEM cross-sectional morphology of wet spun nylon and HnP1-nylon (1, 5, and 10 HnP1 wt. %) fibers.

FIG. 19: SEM surface morphology of wet spun silk and HnP1-silk (1, 2, 5, and 10 wt. % HnP1) fibers,

FIG. 20: SEM cross-sectional morphology of wet spun silk and HnP1-silk (1, 2, 5, and 10 wt. %) fibers.

FIG. 21: Water contact angles (measured in degrees) of wet spun nylon and HnP1-nylon (1, 2, 5, and 10 HnP1 wt. %) fibers.

FIG. 22: Water contact angles (measured in degrees) of wet spun silk and HnP1-silk (1, 2, 5, and 10 HnP1 wt. %) fibers.

### DETAILED DESCRIPTION OF THE INVENTION

### Definitions

The following definitions are presented to better define the present invention and as a guide for those of ordinary skill in the art in the practice of the present invention.

Unless otherwise specified, all technical and scientific terms used herein are to be understood as having the same meanings as is understood by one of ordinary skill in the relevant art to which this disclosure pertains.

The term “comprising” as used in this specification and claims means “consisting at least in part of;” that is to say when interpreting statements in this specification and claims which include “comprising”, the features prefaced by this term in each statement all need to be present but other features can also be present. Related terms such as “comprise” and “comprised” are to be interpreted in a similar manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

The term “vector” as used herein refers to any type of polynucleotide molecule that may be used to manipulate genetic material so that it can be amplified, replicated, manipulated, partially replicated, modified and/or expressed, but not limited thereto. In some embodiments a vector may be used to transport a polynucleotide comprised in that vector into a cell or organism.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, SIRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to nucleic acids, nucleic acid molecules, nucleotide sequences and polynucleotide sequences is to be similarly understood.

The term “gene” as used herein refers to gene, the biological unit of heredity, self-reproducing and located at a definite position (locus) on a particular chromosome. In one embodiment the particular chromosome is a eukaryotic or bacterial chromosome. The term bacterial chromosome is used interchangeably herein with the term bacterial genome,

The term “endogenous” as used herein refers to a constituent of a cell, tissue or organism that originates or is produced naturally within that cell, tissue or organism. An “endogenous” constituent may be any constituent including but not limited to a polynucleotide, a polypeptide including a non-ribosomal polypeptide, a fatty acid or a polyketide, but not limited thereto.

The term “exogenous” as used herein refers to any constituent of a cell, tissue or organism that does not originate or is not produced naturally within that cell, tissue or organism. An exogenous constituent may be, for example, a polynucleotide sequence that has been introduced into a cell, tissue or organism, or a polypeptide expressed in that cell, tissue or organism from that polynucleotide sequence,

“Naturally occurring” as used herein with reference to a polynucleotide sequence according to the invention refers to a primary polynucleotide sequence that is found in nature. A synthetic polynucleotide sequence that is identical to a wild polynucleotide sequence is, for the purposes of this disclosure, considered a naturally occurring sequence. What is important for a naturally occurring polynucleotide sequence is that the actual sequence of nucleotide bases that comprise the polynucleotide is found or known from nature.

For example, a wild-type polynucleotide sequence is a naturally occurring polynucleotide sequence, but not limited thereto. A naturally occurring polynucleotide sequence also refers to variant polynucleotide sequences as found in nature that differ from wild type. For example, allelic variants and naturally occurring recombinant polynucleotide sequences due to hybridization or horizontal gene transfer, but not limited thereto.

“Non-naturally occurring” as used herein with reference to a polynucleotide sequence according to the invention refers to a polynucleotide sequence that is not found in nature. Examples of non-naturally occurring polynucleotide sequences include artificially produced mutant and variant polynucleotide sequences, made for example by point mutation, insertion, or deletion, but not limited thereto. Non-naturally occurring polynucleotide sequences also include chemically evolved sequences. What is important for a non-naturally occurring polynucleotide sequence according to the invention is that the actual sequence of nucleotide bases that comprise the polynucleotide is not found or known from nature.

The term, “wild type” when used herein with reference to a polynucleotide refers to a naturally occurring; non-mutant form of a polynucleotide. A mutant polynucleotide means a polynucleotide that has sustained a mutation as known in the art, such as point mutation, insertion, deletion, substitution, amplification or translocation, but not limited thereto.

The term, “wild type” when used herein with reference to a polypeptide refers to a naturally occurring, non-mutant form of a polypeptide. A wild type polypeptide is a polypeptide that is capable of being expressed from a wild type polynucleotide.

The term “coding sequence” (CDS) or “open reading frame” (ORF) refers to the sense strand of a genomic DNA sequence or a cDNA sequence that is capable of producing a transcription product and/or a polypeptide under the control of appropriate regulatory sequences. The CDS is identified by the presence of a 5′ translation start codon and a 3′ translation stop codon. When inserted into a genetic construct or an expression cassette, a “coding sequence” (CDS) is capable of being expressed when it is operably linked to a promoter sequence and/or other regulatory elements.

“Operably-linked” means that the sequence to be expressed is placed under the control of regulatory elements.

“Regulatory elements” as used herein refers to any nucleic acid sequence element that controls or influences the expression of a polynucleotide insert from a vector, genetic construct or expression cassette and includes promoters, transcription control sequences, translation control sequences, origins of replication, tissue-specific regulatory elements, temporal regulatory elements, enhancers, polyadenylation signals, repressors and terminators. Regulatory elements can be “homologous” or “heterologous” to the polynucleotide insert to be expressed from a genetic construct, expression cassette or vector as described herein. When a genetic construct, expression cassette or vector as described herein is present in a cell, a regulatory element can be “endogenous”, “exogenous”, “naturally occurring” and/or “non-naturally occurring” with respect to the cell.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to non-transcribed cis-regulatory elements upstream of the coding region that regulate the transcription of a polynucleotide sequence. Promoters comprise cis-initiator elements which specify the transcription initiation site and conserved boxes. In one non-limiting example, bacterial promoters may comprise a “Pribnow box” (also known as the −10 region), and other motifs that are bound by transcription factors and promote transcription. Promoters can be homologous or heterologous with respect to polynucleotide sequence to be expressed. When the polynucleotide sequence is to be expressed in a cell, a promoter may be an endogenous or exogenous promoter. Promoters can be constitutive promoters, inducible promoters or regulatable promoters as known in the art.

“Homologous” as used herein with reference to polynucleotide regulatory elements, means a polynucleotide regulatory element that is a native and naturally occurring polynucleotide regulatory element. A homologous polynucleotide regulatory element may be operably linked to a polynucleotide of interest such that the polynucleotide of interest can be expressed from a vector according to the invention.

“Homologous” as used herein with reference to polynucleotide or polypeptide in a host organism means that the polynucleotide or polypeptide is a native and naturally occurring polynucleotide or polynucleotide within that host organism. A homologous polynucleotide may be operably linked to a homologous or heterologous regulatory element so that a homologous polypeptide may be expressed from a vector comprising the homologous polynucleotide as described herein.

“Heterologous” as used herein with reference to polynucleotide regulatory elements, means a polynucleotide regulatory element that is not a native and naturally occurring polynucleotide regulatory element. A heterologous polynucleotide regulatory element is not normally associated with the CDS to which it is operably linked. A heterologous regulatory element may be operably linked to a polynucleotide of interest such that the polynucleotide of interest can be expressed from a polynucleotide or vector according to the invention. Such promoters may include promoters normally associated with other genes, ORFs or coding regions, and/or promoters isolated from any other bacterial, viral, eukaryotic, or mammalian cell.

“Heterologous” as used herein with reference to a polynucleotide or polypeptide in a host organism (i.e., a “heterologous polynucleotide” or “heterologous polypeptide”) means a polynucleotide or polypeptide that is not a native and naturally occurring polynucleotide or polypeptide in that host organism. A heterologous polynucleotide may be operably linked to a heterologous or homologous regulatory element so that a heterologous polypeptide may be expressed from a vector comprising the heterologous polynucleotide as described herein.

The terms “heterologously expressing” and “heterologous expression” mean the expression of a heterologous polypeptide in a host cell.

A “functional variant or fragment thereof” of a polypeptide is a subsequence of the polypeptide that performs a function that is required for the biological activity or binding of that polypeptide and/or provides the three-dimensional structure of the polypeptide. The term may refer to a polypeptide, an aggregate of a polypeptide such as a dimer or other multimer, a fusion polypeptide, a polypeptide fragment, a polypeptide variant, or a functional polypeptide derivative thereof that is capable of performing the polypeptide activity,

“Isolated” as used herein with reference to polynucleotide or polypeptide sequences describes a sequence that has been removed from its natural cellular environment, An isolated molecule may be obtained by any method or combination of methods as known and used in the art, including biochemical, recombinant, and synthetic techniques. The polynucleotide or polypeptide sequences may be prepared by at least one purification step.

“Isolated” when used herein in reference to a cell or host cell describes a cell or host cell that has been obtained or removed from an organism or from its natural environment and is subsequently maintained in a laboratory environment as known in the art. The term encompasses single cells, per se, as well as cells or host cells comprised in a cell culture and can include a single cell or single host cell.

The term “recombinant” refers to a polynucleotide sequence that is removed from sequences that surround it in its natural context and/or is recombined with sequences that are not present in its natural context. A “recombinant” polypeptide sequence is produced by translation from a “recombinant” polynucleotide sequence.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polypeptides useful in the invention have biological activities that are the same or similar to those of a corresponding wild-type molecule, i.e., the parent polypeptides or polynucleotides,

In certain embodiments, variants of the polypeptides described herein have biological activities that are similar, or that are substantially similar to their corresponding wild-type molecules. In certain embodiments the similarities are similar activity and/or binding specificity.

In certain embodiments, variants of polypeptides described herein have biological activities that differ from their corresponding wild-type molecules. In certain embodiments the differences are altered activity and/or binding specificity.

The term “variant” with reference to polynucleotides and polypeptides encompasses all forms of polynucleotides and polypeptides as defined herein.

Variant polynucleotide sequences preferably exhibit at least 50%, at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 8 nucleotide positions, preferably at least 10 nucleotide positions, preferably at least 15 nucleotide positions, preferably at least 20 nucleotide positions, preferably at least 27 nucleotide positions, preferably at least 40 nucleotide positions, preferably at least 50 nucleotide positions, preferably at least 60 nucleotide positions, preferably at least 70 nucleotide positions, preferably at least 80 nucleotide positions, preferably over the entire length of a polynucleotide used in or identified according to a method of the invention.

Polynucleotide variants also encompass those which exhibit a similarity to one or more of the specifically identified sequences that is likely to preserve the functional equivalence of those sequences, and which could not reasonably be expected to have occurred by random chance.

Polynucleotide sequence identity and similarity can be determined readily by those of skill in the art.

Variant polynucleotides also encompass polynucleotides that differ from the polynucleotide sequences described herein but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

The term “variant” with reference to polypeptides also encompasses naturally occurring, recombinantly and synthetically produced polypeptides. Variant polypeptide sequences preferably exhibit at least 35%, preferably at least 40%, preferably at least 50%, preferably at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 2 amino acid positions, preferably at least 3 amino acid positions, preferably at least 4 amino acid positions, preferably at least 5 amino acid positions, preferably at least 7 amino acid positions, preferably at least 10 amino acid positions, preferably at least 15 amino acid positions, preferably at least 20 amino acid positions, preferably over the entire length of a polypeptide used in or identified according to a method of the invention.

Polypeptide variants also encompass those which exhibit a similarity to one or more of the specifically identified sequences that is likely to preserve the functional equivalence of those sequences, and which could not reasonably be expected to have occurred by random chance.

Polypeptide sequence identity and similarity can be determined readily by those of skill in the art.

A variant polypeptide Includes a polypeptide wherein the amino acid sequence differs from a polypeptide herein by one or more conservative amino acid or non-conservative substitutions, deletions, additions, or insertions which do not affect the biological activity of the peptide.

Conservative substitutions typically include the substitution of one amino acid for another with similar characteristics as known and used in the art.

Analysis of evolved biological sequences has shown that not all sequence changes are equally likely, reflecting at least in part the differences in conservative versus non-conservative substitutions at a biological level. For example, certain amino acid substitutions may occur frequently, whereas others are very rare. Evolutionary changes or substitutions in amino acid residues can be modelled by a scoring matrix also referred to as a substitution matrix. Such matrices are used in bioinformatics analysis to identify relationships between sequences and are known to the skilled worker.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g., D-amino acids or non-naturally occurring synthetic amino acids, e.g., beta or gamma amino acids and cyclic analogs.

Substitutions, deletions, additions or insertions may be made by mutagenesis methods known in the art. A skilled worker will be aware of methods for making phenotypically silent amino acid substitutions. See for example Bowie et al., 1990, Science 247, 1306,

The term “peptide” includes chains of amino acids linked by peptide bonds. The term “peptide” can also refer to a “protein” or “polypeptide” which molecules comprising amino acids arranged in a linear chain that may also be folded into a globular form. As used herein, a protein generally refers to a molecule comprising more than about 200 amino acids, up to a full length sequence translated from a gene; a polypeptide generally refers to a molecule comprising more than about 100 amino acids; and a peptide generally refers to a molecule comprising from about 2 to about 100 amino acids. For convenience, the terms “protein,” “polypeptide” and “peptide” are used interchangeably herein. As contemplated herein the protein may represent any macromolecule containing amines or thiols. In this regard, a “protein or peptide” may comprise amino acid sequences comprising at least one of the common amino acids found in naturally occurring proteins, or at least one modified or unusual amino acid. Proteins, polypeptides and peptides, including FUN polypeptides and portions thereof as described herein, can be made by multiple techniques as known to those of skill in the art, including the expression of proteins, polypeptides or peptides through standard molecular biological techniques, the isolation of proteins or peptides from natural sources, or the chemical synthesis of proteins or peptides.

A polypeptide as used herein can also refer to a polypeptide that has been modified during or after synthesis, for example, by biotinylation, benzylation, glycosylation, phosphorylation, amidation, by derivatization using blocking/protecting groups and the like. Such modifications may increase stability or activity of the polypeptide.

The terms “modulate(s) expression”, “modulated expression” and “modulating expression” of a polynucleotide or polypeptide, are intended to encompass the situation where genomic DNA corresponding to a polynucleotide to be expressed according to the invention is modified thus leading to modulated expression of a polynucleotide or polypeptide of the invention. Modification of the genomic DNA may be through genetic transformation or other methods known in the art for inducing mutations. The “modulated expression” can be related to an increase or decrease in the amount of messenger RNA and/or polypeptide produced and may also result in an increase or decrease in the activity of a polypeptide due to alterations in the sequence of a polynucleotide and polypeptide produced.

The terms “modulate(s) activity”, “modulated activity”, and “modulating activity” of a polynucleotide or polypeptide are intended to encompass the situation where genomic DNA corresponding to a polynucleotide to be expressed according to the invention is modified thus leading to modulated expression of a polynucleotide or modulated expression or activity of polypeptide of the invention. Modification of the genomic DNA may be through genetic transformation or other methods known in the art for inducing mutations. The “modulated activity” can be related to an increase or decrease in the amount of messenger RNA and/or polypeptide produced and may also result in an increase or decrease in the functional activity of a polypeptide due to alterations in the sequence of a polynucleotide and polypeptide produced.

The phrase “a polypeptide or portion thereof” in the context of a proteinaceous polymer as described herein refers to a polypeptide as described herein or a portion of that polypeptide that is incorporated into the proteinaceous polymer. The polypeptide or portion thereof comprises a quasi-repeat domain.

The term “quasi-repeat domain” and grammatical variations thereof as used herein refers to an imperfect repeat.

It is intended that reference to a range of numbers disclosed herein (for example 1 to 10) also incorporates reference to all related numbers within that range (for example, 1, 1.1, 2, 3, 3,9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION

The present invention relates generally to a polypeptide from the nesting material protein of the solitary Colletid bee, Hylaeus nubilosus and portions, functional analogs, variants and/or derivatives thereof. The invention also relates generally to a polynucleotide encoding such a polypeptide, a proteinaceous polymer comprising such a polypeptide or portion thereof comprising a quasi-repeat domain, methods of making the polypeptide and a proteinaceous polymer comprising the polypeptide or a portion thereof including by heterologous expression of the polynucleotide in an appropriate isolated host cell.

Bees in the genus Hylaeus (Hymenoptera: Colletidae) produce a nesting material described as ‘cellophane-like’ (Almeida, E. A. B. Colletidae nesting biology (Hymenoptera: Apoidea). Apidologie 39, 16-29 (2008)).

Previously published results on nesting materials produced by closely related bees suggest the material is a unique composite of lipid polymer and protein biopolymers. In an effort to better understand the basis for the observed properties, the inventors undertook an investigation of the nesting material, seeking to identify the constituents of this material that are responsible for its surprising properties.

Molecular analyses of the Dufour's, labial, mandibular glands, and nesting material of H. nubilosus have identified a single major protein component of the bee nesting material of H. nubilosus (>10%), termed FUN_069765-T1 (termed “FUN” polypeptide herein). FUN is a silk-like protein but is not similar or homologous to the silks known to be produced by Hymenoptera (ants, wasps, and bees). The dissimilarity is due to the properties of FUN and the way that FUN is produced. For example, larval honeybees (Apis mellifera) produce silk rich in proteins in their labial glands for end-capping cells prior to pupation. Honeybee silk is made of proteins that predominantly fold into a-helices, which then assemble into larger super secondary structures, coiled coils. The honeybee proteins are composed of four small fibroin subunits ~30 kDa long and comprise ~30% alanine.

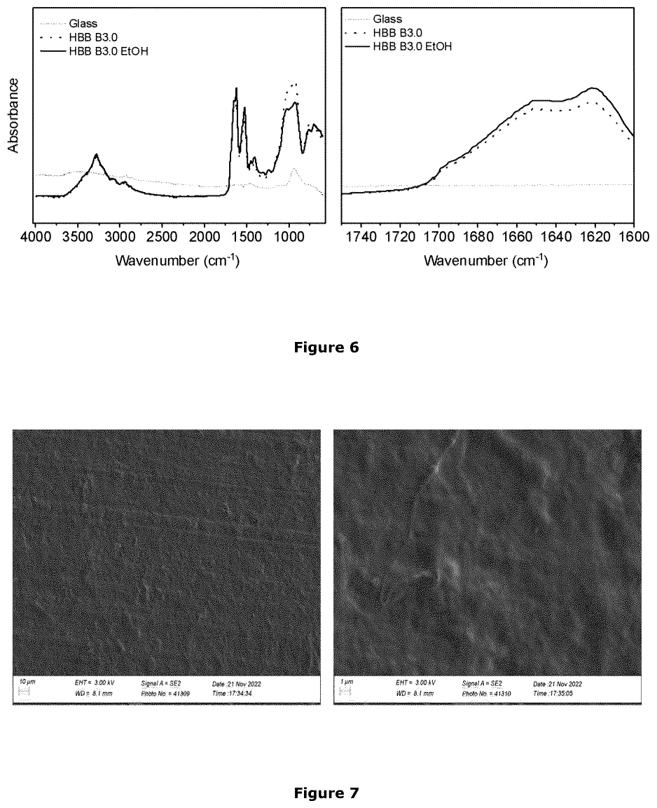

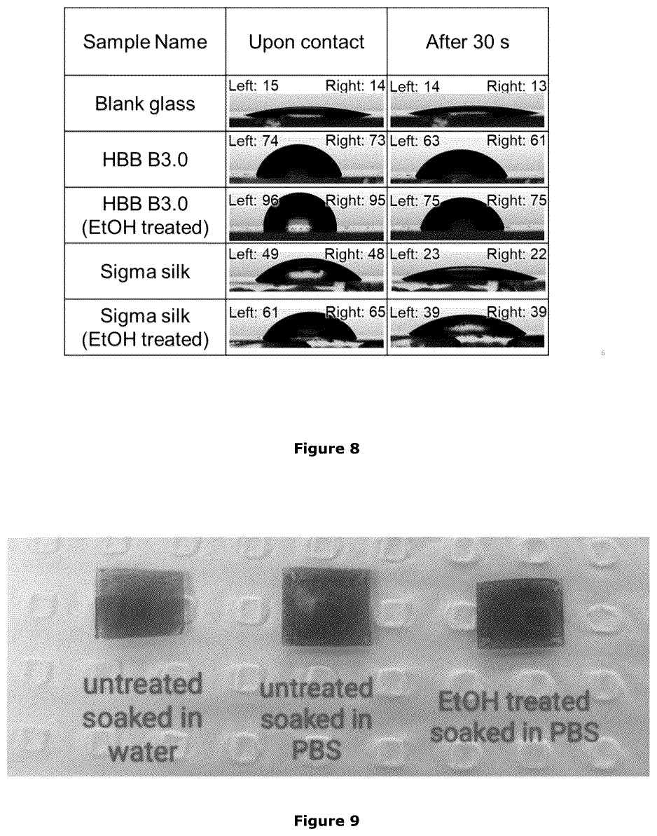

In contrast, FUN from H. nubilosus takes a β-sheet rich structure which is the same structure as in silk fibers made by the silkworm (Bombyx mori) or dragline silk of araneomorph spiders, but different to the α-helical structure that dominates larval honeybee silk (FIGS. 5 and 6). The FUN polypeptide is rich in glutamine and serine, which is not a feature of honeybee silk, silkworm silk, or spider silk, Although one asparagine-rich silk protein (asparagine and glutamine are similar amino acids) has been reported from a distantly related parasitic wasp (Cotesia glomerata), silk proteins rich in glutamine are not known.

Using a combined genomic sequencing, transcriptomic sequencing, and proteomics approach, the inventors have determined the nucleic and amino acid sequences of the FUN polypeptide, including numerous sequences rich in glutamine and serine. Without wishing to be bound by theory, the applicant believes that the FUN polypeptide is comprised in a proteinaceous polymer that is itself comprised in, or comprises, the Hylaeus nubilosus nesting material. Also, without wishing to be bound by theory, the inventors believe that the polypeptide and/or a proteinaceous polymer comprising the polypeptide or at least a portion thereof imparts important structural and functional properties to the Hylaeus nubilosus nest material.

The applicant believes that the FUN polypeptides and variants, analogues and derivatives thereof as described herein, has numerous applications in materials in which known synthetic polymers and biopolymers (including silk proteins) are normally employed.

As described herein, the applicants are the first to provide a silk or silk-like protein from H. nubilosus. To achieve this goal, the applicants analysed the genomic sequence of H. nubilosus and identified a single predicted coding sequence for the major protein associated with the nest material: (CDSs): FUN_069765-T1 genomic DNA (SEQ ID NO: 25) and FUN_069765-T1cDNA (SEQ ID NO: 1), The predicted amino acid sequence of FUN_069765-T1 polypeptide from H. nubilosus is shown in SEQ ID NO: 2. This full-length polypeptide (i.e., SEQ ID NO: 2) as well as other amino acid sub-sequences and functional variants as disclosed herein and described by primary sequence including SEQ ID NO: 4, 6, 8, 10, 12, 14, 16, 18, 20, 22, 24, 50 and 52 (but not necessarily limited thereto) are all referred to herein as FUN polypeptides.

Additionally, polypeptides produced according to SEQ ID NO: 12 as described herein are referred to in this document by different names including: “FSS (FUNSecSf9) with secretion tag cleaved”; “FSS”; “HBB B3.0”; and “HnM1M7-01”. All names describe the same polypeptide and are used synonymously,

Polypeptides produced according to SEQ ID NO:4 as described herein are referred to in this document by “Consensus sequence 43 mer” or “HnP1”. Both names describe the same polypeptide and are used synonymously.

The applicants are also the first to provide methods of heterologous expression of a silk-like protein from H. nubilosus in an isolated host cell.

The gene for the full-length FUN polypeptide from H. nubilosus was first identified by the inventors as described in the examples herein.

An analysis of the predicted coding sequence (i.e., the cDNA) of the full-length FUN polypeptide (SEQ ID NO: 2) using the Evolutionary Failure Mode (EFM) Calculator reveals a highly diverse array of hundreds of repeated DNA motifs, ranging in frequency and length from two copies of a 770 bp motif through to 117 copies of a 16 bp sequence.

The predicted primary amino acid sequence of the full-length FUN polypeptide (SEQ ID NO: 2) reveals a large number of repeated amino acid sequence motifs—both perfect (or identical) repeats and imperfect (or quasi-) repeats—that are organised in a complex fashion. The RADAR (Rapid Automatic Detection and Alignment of Repeats) tool, which allows for mismatches and gaps, identifies several categories of repeat, but the imperfect and fragmented nature of many of these repeats leads to a complex picture of the overall architecture of this protein,

Using these initial surveys as a basis for a more extensive analysis it appears that almost the entire protein can be organised as a consecutive series of peptide domains (which are also termed FUN polypeptides herein) each built around a fundamental, repeating 43 amino acid motif that is found exclusively in the QS-repeat regions and has the following consensus (with a >50% threshold):

(SEQ ID NO: 6)
SESAVQSSKSSSGMSSQAQSQQQQX₁QLQQSQAQSQEQESQSAX₂.

In some embodiments X₁ and X₂ are independently any amino acid. By accommodating a limited number of substitutions at key residues as well as gaps, the entire protein, apart from the first 137 amino acids (the non-repetitive region) and the spacer repeats, can be organised according to this repeating 43-mer consensus,

In one embodiment X₁=A or S. In one embodiment X₂=E, G or Q.

Seventy-five peptides from the QS-repeat regions conform to a strict 43 residue length of the consensus and these seventy-five repeats are designated as Class A repeats.

Interspersed amongst these Class A repeats are shorter versions of the consensus, also located in the QS-repeat regions, that are 35-36 residues in length, which can be broadly categorised into two classes; those having a tetra-glutamine (QQQQ) motif (designated Class B) and confined to the second QS-repeat region, see for example amino acid residues 635-670 of SEQ ID NO: 2 having the amino acid sequence:

(SEQ ID NO: 18)
SESAVQSSKSSSGMSSQAQSQQQQAQSQEQESQSAQ,

and those lacking the tetra-glutamine motif (Class C), found scattered throughout the molecule, see for example amino acid residues 1508-1543 of SEQ ID NO: 2 having the amino acid sequence:

(SEQ ID NO: 20)
SESDVQSSKSSRGMSSHAQSQQSQAQSQEQESQSAQ.

The Class A, B and C repeats are, by definition, found exclusively in the QS-repeat regions, which are interspersed with a small number of irregular repeats, designated as spacer repeats (Class X), which can be aligned to each other, but cannot be aligned to the QS-repeat region sequences. These irregular spacer repeats contain the majority of the proline and cysteine residues found in the protein.

Except for the final spacer repeat, which forms the C-terminus of the protein, all other spacer repeats are followed by a variable length repeat, ranging from 23 to 38 residues in length, designated Class D, for example, amino acid residues 3087-3109 of SEQ ID NO: 2 having the amino acid sequence: MSSRAQSQQSQAQSQEQESQSAQ (SEQ ID NO: 22).

Also, the spacer repeats are all preceded by another variable length repeat, ranging from 5 to 40 residues in length, designated Class E; see for example amino acid residues 2249-2259 of SEQ ID NO: 2 having the amino acid sequence: SESAVQSSKSS (SEQ ID NO: 24).

Despite their highly variable lengths, both Class D and E repeats can be aligned to the 43-mer consensus.

The repeat region of the FUN polypeptide comprises amino acid residues 138 to 5451 of SEQ ID NO: 2 and is organised as a series of consecutive peptide domains aligned to the 43-mer and spacer repeat consensus motifs.

Immediately following the non-repetitive N-terminal domain (NTD or N, amino acid residues 18-137 of SEQ ID NO: 2) is a 21-mer peptide (amino acid residues 138-158 of SEQ ID NO: 2) that, while it cannot be assigned to any of the other repeat classes, is still able to be aligned with the 43-mer consensus (designated Class F).

### Protein Synthesis

A FUN polypeptide and/or various portions thereof as described herein can be made by chemical synthesis using methods such as solution phase synthesis or solid phase peptide synthesis, with or without chemical ligation, followed by purification of the resulting peptides. In some embodiments the FUN polypeptide or portion thereof as described herein is made by chemical synthesis.

### Protein Expression

To confirm the function and characterize the properties of the FUN polypeptide, the inventors constructed a series of expression vectors. These vectors were transformed into an appropriate host for the heterologous production of FUN_069765-T1 protein.

Following transformation and expression of the FUN polypeptide and various portions thereof as described herein in an appropriate host, the inventors determined the chemical phenotypes of the transformants initially by normal-phase thin-layer chromatography (TLC, results not shown) and subsequently by reversed-phase liquid chromatography-mass spectrometry (LC-MS) analysis of cell extracts. The inventors purified the newly expressed metabolites, as determined by high-resolution mass spectrometry (HRMS), by semi-preparative reversed-phase high-performance liquid chromatography (HPLC) and subjected compounds to nuclear magnetic resonance (NMR) spectroscopic analysis (1H, 13C, and HSQC, HMBC, COSY) for final identification.

Based on the work described herein, the inventors disclose the use of heterologous expression to produce recombinant FUN polypeptides. Overall, the inventors' work described herein confirms that heterologous expression of at least a portion of the complete FUN polypeptide from H. nubilosus in a heterologous host is a viable method that can be employed to produce this polypeptide, including relevant portions, variants, analogues, and derivatives thereof artificially using a recombinant biosynthetic system. As noted above, the polypeptides described herein by SEQ ID NO: including SEQ ID NO: 2, 4, 6, 8, 10, 12, 14, 16, 18, 20, 22, 24, 50 and 52 are all termed FUN polypeptides.

In one aspect the present invention relates to an isolated polynucleotide encoding a polypeptide comprising at least 70% amino acid sequence identity to SEQ ID NO: 2.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 2.

In one embodiment the isolated polynucleotide encodes a polypeptide comprising the amino acid sequence of SEQ ID NO: 2. In one embodiment the isolated polynucleotide encodes a polypeptide consisting essentially of or consisting of the amino acid sequence of SEQ ID NO: 2

In one embodiment the polypeptide comprises from 1 to 75 copies of SEQ ID NO: 6.

In one embodiment the polypeptide comprises at least one copy of SEQ ID NO: 18.

In one embodiment the polypeptide comprises at least one copy of SEQ ID NO: 20.

In one embodiment the polypeptide comprises at least one copy of a SEQ ID NO: 22.

In one embodiment the polypeptide comprises at least one copy of SEQ ID NO: 24.

In one embodiment the isolated polypeptide comprises at least one copy of SEQ ID NO: 52.

In one embodiment the polypeptide comprises at least two copies of SEQ ID NO: 6 separated by at least one copy of SEQ ID NO: 24 and at least one copy of SEQ ID NO: 22 In one embodiment the polypeptide has the following organization SEQ ID NO: 6 OR SEQ ID NO: 20-SEQ ID NO:24-SPACER-SEQ ID NO:22-SEQ ID NO: 6 OR SEQ ID NO:20).

In one embodiment the isolated polypeptide comprises, consists essentially of or consists of at least 25% serine, 25% glutamine and 5% glutamic acid residues.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 1. In one embodiment the isolated polynucleotide comprises, consists, or consists essentially of SEQ ID NO: 1.

In another aspect the invention relates to an isolated polynucleotide encoding a polypeptide comprising SEQ ID NO: 2.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 1. In one embodiment the isolated polynucleotide comprises, consists, or consists essentially of SEQ ID NO: 1.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 2.

The isolated polynucleotide molecules described herein can be isolated from a biological sample using a variety of techniques known to those of ordinary skill in the art. By way of example, such polynucleotides can be isolated through use of the polymerase chain reaction (PCR) as known in the art. The nucleic acid molecules described can be amplified using primers, as defined herein, derived from the polynucleotide sequences as described herein.

Further methods for isolating polynucleotides include the use of all, or portions of, a polynucleotide as described herein as hybridization probes. The technique of hybridizing labelled polynucleotide probes to polynucleotides immobilized on solid supports such as nitrocellulose filters or nylon membranes, can be used to screen genomic or cDNA libraries. Similarly, probes may be coupled to beads and hybridized to the target sequence. Isolation can be affected using known art protocols such as magnetic separation. The choice of appropriately stringent hybridization and wash conditions is believed to be within the skill of those in the art.

Polynucleotide fragments may be produced by techniques well-known in the art, such as restriction endonuclease digestion and oligonucleotide synthesis.

A partial polynucleotide sequence may be used as a probe, in methods well-known in the art to identify the corresponding full-length polynucleotide sequence in a sample. Such methods include PCR-based methods, 5′RACE and hybridization-based methods, and computer/database-based methods as known in the art. Detectable labels such as radioisotopes, fluorescent, chemiluminescent and bioluminescent labels may be used to facilitate detection. Inverse PCR also permits the acquisition of unknown sequences, flanking the polynucleotide sequences disclosed herein, starting with primers based on a known region as known and used in the art. The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized as known in the art. Primers and primer pairs which allow amplification of polynucleotides of the invention, are also contemplated as embodiments disclosed herein.

Variants (including orthologues) may be identified by the methods described. Variant polynucleotides may be identified using PCR-based methods as known in the art. Typically, the polynucleotide sequence of a primer, useful to amplify variants of polynucleotide molecules by PCR, may be based on a sequence encoding a conserved region of the corresponding amino acid sequence,

Further methods for identifying variant polynucleotides include the use of all or portions of the specified polynucleotides as hybridization probes to screen genomic or cDNA libraries as described above. Typically probes based on a sequence encoding a conserved region of the corresponding amino acid sequence may be used. Hybridization conditions may also be less stringent than those used when screening for sequences identical to the probe.

In another aspect, the invention relates to an isolated polypeptide comprising at least 70% amino acid sequence identity to SEQ ID NO: 2.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 2.

In one embodiment the isolated polypeptide comprises SEQ ID NO: 2. In one embodiment the isolated polypeptide consists or consists essentially of SEQ ID NO: 2.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the isolated polypeptide comprises from 1 to 75 copies of SEQ ID NO: 6.

In one embodiment the isolated polypeptide comprises at least one copy of SEQ ID NO: 18.

In one embodiment the isolated polypeptide comprises at least one copy of SEQ ID NO: 20.

In one embodiment the isolated polypeptide comprises at least one copy of a SEQ ID NO: 22.

In one embodiment the isolated polypeptide comprises at least one copy of SEQ ID NO: 24.

In one embodiment the isolated polypeptide comprises at least one copy of SEQ ID NO: 52.

In one embodiment the polypeptide comprises at least two copies of SEQ ID NO: 6 separated by at least one copy of SEQ ID NO: 24 and at least one copy of SEQ ID NO: 22 In one embodiment the polypeptide has the following organisation SEQ ID NO: 6 OR SEQ ID NO: 20-SEQ ID NO:24-SPACER-SEQ ID NO:22-SEQ ID NO: 6 OR SEQ ID NO:20).

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 1. In one embodiment the isolated polynucleotide comprises, consists or consists essentially of SEQ ID NO: 1.

In one embodiment the nucleic acid sequence further comprises a heterologous regulatory element. In one embodiment the regulatory element comprises a nucleic acid sequence encoding a signal peptide.

In another aspect the present invention relates to an isolated polynucleotide encoding a polypeptide comprising at least 70% amino acid sequence identity to

(SEQ ID NO: 6)
SESAVQSSKSSSGMSSQAQSQQQQX₁QLQQSQAQSQEQESQSAX₂.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 6.

In one embodiment the isolated polynucleotide encodes a polypeptide comprising the amino acid sequence of SEQ ID NO: 6. In one embodiment the isolated polynucleotide encodes a polypeptide consisting essentially of or consisting of the amino acid sequence of SEQ ID NO: 6

In one embodiment X₁ and X₂ are independently any amino acid. In one embodiment X₁=A or S. In one embodiment X₂=E, G or Q.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the isolated polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 6. In one embodiment the polymer comprises between two and 75 copies of SEQ ID NO: 6.

In one embodiment the polypeptide comprises at least two copies of SEQ ID NO: 6 separated by at least one copy of SEQ ID NO: 24 and at least one copy of SEQ ID NO: 22 In one embodiment the polypeptide has following organization SEQ ID NO: 6 OR SEQ ID NO: 20-SEQ ID NO:24-SPACER-SEQ ID NO:22-SEQ ID NO: 6 OR SEQ ID NO:20).

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 5. In one embodiment the isolated polynucleotide comprises, consists, or consists essentially of SEQ ID NO: 5.

In another aspect the invention relates to an isolated polynucleotide encoding a polypeptide comprising SESAVQSSKSSSGMSSQAQSQQQQX₁QLQQSQAQSQEQESQSAX₂ (SEQ ID NO: 6). In one embodiment X₁ or X₂ are independently any amino acid. In one embodiment X₁=A or S. In one embodiment X₂=E, G or Q.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 6. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to

(SEQ ID NO: 6)
SESAVQSSKSSSGMSSQAQSQQQQX₁QLQQSQAQSQEQESQSAX₂.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 6.

In one embodiment the isolated polypeptide comprises SEQ ID NO: 6. In one embodiment the isolated polypeptide consists or consists essentially of SEQ ID NO: 6.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 6. In one embodiment the polymer comprises between two and 75 copies of SEQ ID NO: 6,

In one embodiment the polypeptide comprises at least two copies of SEQ ID NO: 6 separated by at least one copy of SEQ ID NO: 24 and at least one copy of SEQ ID NO: 22 In one embodiment the polypeptide has following organization SEQ ID NO: 6 OR SEQ ID NO: 20-SEQ ID NO:24-SPACER-SEQ ID NO:22-SEQ ID NO: 6 OR SEQ ID NO:20.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 5. In one embodiment the isolated polynucleotide comprises, consists, or consists essentially of SEQ ID NO: 5.

In another aspect the invention relates to an isolated polynucleotide encoding a polypeptide comprising at least 70% amino acid sequence identity to

(SEQ ID NO: 4)
MSSQAQSQQQQSQLQQSQAQSQEQESQSAX₁SGSDVX₂X₃SKSSSG.

In one embodiment X₁, X₂ and X₃ is independently any amino acid. In one embodiment X₁=G or Q. In one embodiment X₂=Q or E. In one embodiment X₃=S or V.

In one embodiment the isolated polynucleotide encodes a polypeptide comprising at least 75%, preferably at least 80%, preferably at least 85%, preferably at least 90%, preferably at least 95%, preferably at least 99% amino acid sequence identity to SEQ ID NO: 4.

In one embodiment the isolated polynucleotide encodes a polypeptide comprising the amino acid sequence of SEQ ID NO: 4. In one embodiment the isolated polynucleotide encodes a polypeptide consisting essentially of or consisting of the amino acid sequence of SEQ ID NO: 4.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 4.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 3. In one embodiment the isolated polynucleotide comprises, consists, or consists essentially of SEQ ID NO: 3.

In another aspect the invention relates to an isolated polynucleotide encoding a polypeptide comprising MSSQAQSQQQQSQLQQSQAQSQEQESQSAX₁SGSDVX₂X₃SKSSSG (SEQ ID NO: 4). In one embodiment X₁, X₂ and X₃ is independently any amino acid. In one embodiment X₁=G or Q. In one embodiment X₂=Q or E. In one embodiment X₃=S or V.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 4. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to

(SEQ ID NO: 4)
MSSQAQSQQQQSQLQQSQAQSQEQESQSAX₁SGSDVX₂X₃SKSSSG.

In one embodiment X₁, X₂ and X₃ is independently any amino acid. In one embodiment X₁=G or Q. In one embodiment X₂=Q or E. In one embodiment X₃=S or V.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 4.

In one embodiment the isolated polypeptide comprises SEQ ID NO: 4. In one embodiment the isolated polypeptide consists or consists essentially of SEQ ID NO: 4.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 6.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 3. In one embodiment the isolated polynucleotide comprises, consists or consists essentially of SEQ ID NO: 3.

In another aspect the invention relates to an isolated polynucleotide encoding a polypeptide comprising at least 70% amino acid sequence identity to SEQ ID NO: 8.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 8.

In one embodiment the isolated polynucleotide encodes a polypeptide comprising the amino acid sequence of SEQ ID NO: 8. In one embodiment the isolated polynucleotide encodes a polypeptide consisting essentially of or consisting of the amino acid sequence of SEQ ID NO: 8.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 8.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 7. In one embodiment the isolated polynucleotide comprises, consists, or consists essentially of SEQ ID NO: 7.

In another aspect the invention relates to an isolated polynucleotide encoding a polypeptide comprising SEQ ID NO: 8.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 8. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 8

In one embodiment the isolated polypeptide comprises at least 75%, preferably at least 80%, preferably at least 85%, preferably at least 90%, preferably at least 95%, preferably at least 99% amino acid sequence identity to SEQ ID NO: 8.

In one embodiment the isolated polypeptide comprises SEQ ID NO: 8. In one embodiment the isolated polypeptide consists or consists essentially of SEQ ID NO: 8.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 8,

In one embodiment the isolated polypeptide is encoded by a nucleic acid sequence comprising at least 75%, preferably at least 80%, preferably at least 85%, preferably at least 90%, preferably at least 95%, preferably at least 99% nucleic acid sequence identity to SEQ ID NO: 7. In one embodiment the isolated polynucleotide comprises, consists, or consists essentially of SEQ ID NO: 7.

In another aspect the invention relates to an isolated polynucleotide encoding a polypeptide comprising at least 70% amino acid sequence identity to SEQ ID NO: 10.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 10.

In one embodiment the isolated polynucleotide encodes a polypeptide comprising the amino acid sequence of SEQ ID NO: 10. In one embodiment the isolated polynucleotide encodes a polypeptide consisting essentially of or consisting of the amino acid sequence of SEQ ID NO: 10.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 10.

In one embodiment the isolated polypeptide is encoded by a nucleic acid sequence comprising at least 75%, preferably at least 80%, preferably at least 85%, preferably at least 90%, preferably at least 95%, preferably at least 99% nucleic acid sequence identity to SEQ ID NO: 9. In one embodiment the isolated polynucleotide comprises, consists, or consists essentially of SEQ ID NO: 9.

In another aspect the invention relates to an isolated polynucleotide encoding a polypeptide comprising SEQ ID NO: 10.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 10. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 10,

In one embodiment the isolated polypeptide comprises at least 75%, preferably at least 80%, preferably at least 85%, preferably at least 90%, preferably at least 95%, preferably at least 99% amino acid sequence identity to SEQ ID NO: 10.

In one embodiment the isolated polypeptide comprises SEQ ID NO: 10. In one embodiment the isolated polypeptide consists or consists essentially of SEQ ID NO: 10.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 10.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 9. In one embodiment the isolated polynucleotide comprises, consists, or consists essentially of SEQ ID NO: 9.

In another aspect the invention relates to an isolated polynucleotide encoding a polypeptide comprising at least 70% amino acid sequence identity to (SEQ ID NO: 12).

In one embodiment the isolated polynucleotide encodes a polypeptide comprising at least 75%, preferably at least 80%, preferably at least 85%, preferably at least 90%, preferably at least 95%, preferably at least 99% amino acid sequence identity to SEQ ID NO: 12.

In one embodiment the isolated polynucleotide encodes a polypeptide comprising the amino acid sequence of SEQ ID NO: 12. In one embodiment the isolated polynucleotide encodes a polypeptide consisting essentially of or consisting of the amino acid sequence of SEQ ID NO: 12.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 12.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 11. In one embodiment the isolated polynucleotide comprises, consists, or consists essentially of SEQ ID NO: 11.

In another aspect the invention relates to an isolated polynucleotide encoding a polypeptide comprising (SEQ ID NO: 12).

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 12. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to (SEQ ID NO: 12).

In one embodiment the isolated polypeptide comprises at least 75%, preferably at least 80%, preferably at least 85%, preferably at least 90%, preferably at least 95%, preferably at least 99% amino acid sequence identity to SEQ ID NO: 12.

In one embodiment the isolated polypeptide comprises SEQ ID NO: 12. In one embodiment the isolated polypeptide consists or consists essentially of SEQ ID NO: 12.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 12.

In one embodiment the isolated polypeptide is encoded by a nucleic acid sequence comprising at least 75%, preferably at least 80%, preferably at least 85%, preferably at least 90%, preferably at least 95%, preferably at least 99% nucleic acid sequence identity to SEQ ID NO: 11. In one embodiment the isolated polynucleotide comprises, consists, or consists essentially of SEQ ID NO: 11.

In another aspect the invention relates to an isolated polynucleotide encoding a polypeptide comprising at least 70% amino acid sequence identity to SEQ ID NO: 14.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 14.

In one embodiment the isolated polynucleotide encodes a polypeptide comprising the amino acid sequence of SEQ ID NO: 14. In one embodiment the isolated polynucleotide encodes a polypeptide consisting essentially of or consisting of the amino acid sequence of SEQ ID NO: 14.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2.

In one embodiment the isolated polypeptide is encoded by a nucleic acid sequence comprising at least 75%, preferably at least 80%, preferably at least 85%, preferably at least 90%, preferably at least 95%, preferably at least 99% nucleic acid sequence identity to SEQ ID NO: 13. In one embodiment the isolated polynucleotide comprises, consists, or consists essentially of SEQ ID NO: 13.

In another aspect the invention relates to an isolated polynucleotide encoding a polypeptide comprising SEQ ID NO: 14.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 14. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 14.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 14.

In one embodiment the isolated polypeptide comprises SEQ ID NO: 14. In one embodiment the isolated polypeptide consists or consists essentially of SEQ ID NO: 14.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 14.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 13. In one embodiment the isolated polynucleotide comprises, consists, or consists essentially of SEQ ID NO: 13.

In another aspect the invention relates to an isolated polynucleotide encoding a polypeptide comprising at least 70% amino acid sequence identity to SEQ ID NO: 16.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 16.

In one embodiment the isolated polynucleotide encodes a polypeptide comprising the amino acid sequence of SEQ ID NO: 16. In one embodiment the isolated polynucleotide encodes a polypeptide consisting essentially of or consisting of the amino acid sequence of SEQ ID NO: 16.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 16.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 15. In one embodiment the isolated polynucleotide comprises, consists, or consists essentially of SEQ ID NO: 15.

In another aspect the invention relates to an isolated polynucleotide encoding a polypeptide comprising SEQ ID NO: 16.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 16. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 16.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 16.

In one embodiment the isolated polypeptide comprises SEQ ID NO: 16. In one embodiment the isolated polypeptide consists or consists essentially of SEQ ID NO: 16,

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 16.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 15. In one embodiment the isolated polynucleotide comprises, consists, or consists essentially of SEQ ID NO: 15.

In another aspect the present invention relates to an isolated polynucleotide encoding a polypeptide comprising at least 70% amino acid sequence identity to SEQ ID NO: 18.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 18.

In one embodiment the isolated polynucleotide encodes a polypeptide comprising the amino acid sequence of SEQ ID NO: 18. In one embodiment the isolated polynucleotide encodes a polypeptide consisting essentially of or consisting of the amino acid sequence of SEQ ID NO: 18.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the isolated polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 18. In one embodiment the polymer comprises between two and 6 copies of SEQ ID NO: 18.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 17. In one embodiment the isolated polynucleotide comprises, consists, or consists essentially of SEQ ID NO: 17.

In another aspect the invention relates to an isolated polynucleotide encoding a polypeptide comprising SEQ ID NO: 18.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 18. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 18.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 18.

In one embodiment the isolated polypeptide comprises SEQ ID NO: 18. In one embodiment the isolated polypeptide consists or consists essentially of SEQ ID NO: 18.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 18. In one embodiment the polymer comprises between two and 6 copies of SEQ ID NO: 18.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 17. In one embodiment the isolated polynucleotide comprises, consists, or consists essentially of SEQ ID NO: 17.

In another aspect the present invention relates to an isolated polynucleotide encoding a polypeptide comprising at least 70% amino acid sequence identity to SEQ ID NO: 20.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 20.

In one embodiment the isolated polynucleotide encodes a polypeptide comprising the amino acid sequence of SEQ ID NO: 20. In one embodiment the isolated polynucleotide encodes a polypeptide consisting essentially of or consisting of the amino acid sequence of SEQ ID NO: 20.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the isolated polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 20. In one embodiment the polymer comprises between two and 29 of SEQ ID NO: 20.

In one embodiment the polypeptide comprises at least two copies of SEQ ID NO: 20 separated by at least one copy of SEQ ID NO: 24 and at least one copy of SEQ ID NO: 22

In one embodiment the polypeptide has the following structure SEQ ID NO: 6 OR SEQ ID NO: 20-SEQ ID NO:24-SPACER-SEQ ID NO:22-SEQ ID NO: 6 OR SEQ ID NO: 20.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 19. In one embodiment the isolated polynucleotide comprises, consists, or consists essentially of SEQ ID NO: 19.

In another aspect the invention relates to an isolated polynucleotide encoding a polypeptide comprising SEQ ID NO: 20.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 20. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 20,

In one embodiment the isolated polypeptide comprises at least 75%, preferably at least 80%, preferably at least 85%, preferably at least 90%, preferably at least 95%, preferably at least 99% amino acid sequence identity to SEQ ID NO: 20.

In one embodiment the isolated polypeptide comprises SEQ ID NO: 20. In one embodiment the isolated polypeptide consists or consists essentially of SEQ ID NO: 20.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2.

In one embodiment the polymer comprises at least two copies of SEQ ID NO: 20. In one embodiment the polymer comprises between two and 29 of SEQ ID NO: 20.

In one embodiment the polypeptide comprises at least two copies of SEQ ID NO: 20 separated by at least one copy of SEQ ID NO: 24 and at least one copy of SEQ ID NO: 22 In one embodiment the polypeptide has the following structure SEQ ID NO: 6 OR SEQ ID NO: 20-SEQ ID NO:24-SPACER-SEQ ID NO:22-SEQ ID NO: 6 OR SEQ ID NO:20.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 19. In one embodiment the isolated polynucleotide comprises, consists, or consists essentially of SEQ ID NO: 19.

In another aspect the present invention relates to an isolated polynucleotide encoding a polypeptide comprising at least 70% amino acid sequence identity to SEQ ID NO: 22.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 22,

In one embodiment the isolated polynucleotide encodes a polypeptide comprising the amino acid sequence of SEQ ID NO: 22. In one embodiment the isolated polynucleotide encodes a polypeptide consisting essentially of or consisting of the amino acid sequence of SEQ ID NO: 22.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the isolated polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 22. In one embodiment the polymer comprises between two and 10 copies of SEQ ID NO: 22.

In one embodiment the isolated polypeptide is comprised in a proteinaceous polymer, the polymer comprising at least one copies of SEQ ID NO: 22 located between two copies of SEQ ID NO: 6.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 21. In one embodiment the isolated polynucleotide comprises, consists, or consists essentially of SEQ ID NO: 21.

In another aspect the invention relates to an isolated polynucleotide encoding a polypeptide comprising SEQ ID NO: 22.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 22. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 22.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 22.

In one embodiment the isolated polypeptide comprises SEQ ID NO: 22. In one embodiment the isolated polypeptide consists or consists essentially of SEQ ID NO: 22.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2.

In one embodiment the polymer comprises at least two copies of SEQ ID NO: 22. In one embodiment the polymer comprises between two and 10 of SEQ ID NO: 22.

In one embodiment the isolated polypeptide is comprised in a proteinaceous polymer, the polymer comprising at least one copy of SEQ ID NO: 22 located between at two copies of SEQ ID NO: 6.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 21. In one embodiment the isolated polynucleotide comprises, consists, or consists essentially of SEQ ID NO: 21.

In another aspect the present invention relates to an isolated polynucleotide encoding a polypeptide comprising at least 70% amino acid sequence identity to SEQ ID NO: 24.

In one embodiment the isolated polynucleotide encodes a polypeptide comprising at least at least 75%, preferably at least 80%, preferably at least 85%, preferably at least 90%, preferably at least 95%, preferably at least 99% amino acid sequence identity to SEQ ID NO: 24.

In one embodiment the isolated polynucleotide encodes a polypeptide comprising the amino acid sequence of SEQ ID NO: 24. In one embodiment the isolated polynucleotide encodes a polypeptide consisting essentially of or consisting of the amino acid sequence of SEQ ID NO: 24.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the isolated polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 24. In one embodiment the polymer comprises between two and 11 copies of SEQ ID NO: 24.

In one embodiment the isolated polypeptide is comprised in a proteinaceous polymer, the polymer comprising at least one copies of SEQ ID NO: 24 located between two copies of SEQ ID NO: 6.

In one embodiment the isolated polynucleotide comprises a nucleic acid sequence comprising at least at least 75%, preferably at least 80%, preferably at least 85%, preferably at least 90%, preferably at least 95%, preferably at least 99% nucleic acid sequence identity to SEQ ID NO: 23. In one embodiment the isolated polynucleotide comprises, consists, or consists essentially of SEQ ID NO: 23.

In another aspect the invention relates to an isolated polynucleotide encoding a polypeptide comprising SEQ ID NO: 24.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 24. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to SEQ ID NO: 24.

In one embodiment the isolated polypeptide comprises at least 75%, preferably at least 80%, preferably at least 85%, preferably at least 90%, preferably at least 95%, preferably at least 99% amino acid sequence identity to SEQ ID NO: 24.

In one embodiment the isolated polypeptide comprises SEQ ID NO: 24. In one embodiment the isolated polypeptide consists or consists essentially of SEQ ID NO: 24.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2.

In one embodiment the polymer comprises at least two copies of SEQ ID NO: 24. In one embodiment the polymer comprises between two and 11 of SEQ ID NO: 24.

In one embodiment the isolated polypeptide is comprised in a proteinaceous polymer, the polymer comprising at least one copy of SEQ ID NO: 24 located between at two copies of SEQ ID NO: 6.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 23. In one embodiment the isolated polynucleotide comprises, consists or consists essentially of SEQ ID NO: 23.

Specifically contemplated as embodiments of each of the isolated polynucleotide aspects set forth above, the isolated polynucleotide further comprises a heterologous regulatory element. In one embodiment the regulatory element comprises a nucleic acid sequence encoding a signal peptide. Transcription of the polynucleotide comprising a nucleic acid sequence encoding a signal peptide allows for the secretion of the expressed polypeptide and/or proteinaceous polymer from a host cell.

In another aspect the invention relates to an isolated polynucleotide encoding a polypeptide comprising at least 70% amino acid sequence identity to (SEQ ID NO: 50).

In one embodiment the isolated polynucleotide encodes a polypeptide comprising at least 75%, preferably at least 80%, preferably at least 85%, preferably at least 90%, preferably at least 95%, preferably at least 99% amino acid sequence identity to SEQ ID NO: 50.

In one embodiment the isolated polynucleotide encodes a polypeptide comprising the amino acid sequence of SEQ ID NO: 50. In one embodiment the isolated polynucleotide encodes a polypeptide consisting essentially of or consisting of the amino acid sequence of SEQ ID NO: 50.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 50.

In one embodiment the isolated polynucleotide comprises a nucleic acid sequence comprising at least 75%, preferably at least 80%, preferably at least 85%, preferably at least 90%, preferably at least 95%, preferably at least 99% nucleic acid sequence identity to SEQ ID NO: 49. In one embodiment the isolated polynucleotide comprises, consists, or consists essentially of SEQ ID NO: 49.

In one embodiment the polynucleotide further encodes a His-tag. In one embodiment the His tag is at the N-terminus or the C-terminus of the polypeptide.

In another aspect the invention relates to an isolated polynucleotide encoding a polypeptide comprising (SEQ ID NO: 50). In one embodiment the isolated polynucleotide encodes a polypeptide consisting essentially of or consisting of SEQ ID NO: 50.

In one embodiment the polynucleotide further encodes a His-tag. In one embodiment the His tag is at the N-terminus or the C-terminus of the polypeptide.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 50. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to (SEQ ID NO: 50).

In one embodiment the isolated polypeptide comprises at least 75%, preferably at least 80%, preferably at least 85%, preferably at least 90%, preferably at least 95%, preferably at least 99% amino acid sequence identity to SEQ ID NO: 50.

In one embodiment the isolated polypeptide comprises SEQ ID NO: 50. In one embodiment the Isolated polypeptide consists or consists essentially of SEQ ID NO: 50.

In one embodiment the polynucleotide further encodes a His-tag. In one embodiment the His tag is at the N-terminus or the C-terminus of the polypeptide.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 6.

In one embodiment the isolated polypeptide is encoded by a nucleic acid sequence comprising at least at least 75%, preferably at least 80%, preferably at least 85%, preferably at least 90%, preferably at least 95%, preferably at least 99% nucleic acid sequence identity to SEQ ID NO: 49. In one embodiment the isolated polynucleotide comprises, consists or consists essentially of SEQ ID NO: 49.

In one embodiment the polynucleotide further encodes a His-tag. In one embodiment the His tag is at the N-terminus or the C-terminus of the polypeptide.

In another aspect the invention relates to an isolated polynucleotide encoding a polypeptide comprising at least 70% amino acid sequence identity to (SEQ ID NO: 52).

In one embodiment the isolated polynucleotide encodes a polypeptide comprising at least 75%, preferably at least 80%, preferably at least 85%, preferably at least 90%, preferably at least 95%, preferably at least 99% amino acid sequence identity to SEQ ID NO: 52.

In one embodiment the isolated polynucleotide encodes a polypeptide comprising the amino acid sequence of SEQ ID NO: 52. In one embodiment the isolated polynucleotide encodes a polypeptide consisting essentially of or consisting of the amino acid sequence of SEQ ID NO: 52.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 52.

In one embodiment the isolated polynucleotide comprises a nucleic acid sequence comprising at least 75%, preferably at least 80%, preferably at least 85%, preferably at least 90%, preferably at least 95%, preferably at least 99% nucleic acid sequence identity to SEQ ID NO: 51. In one embodiment the isolated polynucleotide comprises, consists, or consists essentially of SEQ ID NO: 51.

In one embodiment the polynucleotide further encodes a His-tag. In one embodiment the His tag is at the N-terminus or the C-terminus of the polypeptide.

In another aspect the invention relates to an isolated polynucleotide encoding a polypeptide comprising (SEQ ID NO: 52). In one embodiment the isolated polynucleotide encodes a polypeptide consisting essentially of or consisting of SEQ ID NO: 52.

In one embodiment the polynucleotide further encodes a His-tag. In one embodiment the His tag is at the N-terminus or the C-terminus of the polypeptide.

Specifically contemplated as embodiments of this aspect of the invention are all of the embodiments set forth in the previous aspects of the invention directed to the isolated polynucleotide encoding a polypeptide comprising at least 70% amino acid sequence identity to SEQ ID NO: 52. The skilled person also recognizes that the disclosure above related to an isolated polynucleotide comprising, consisting essentially of or consisting of SEQ ID NO: 1 is equally and appropriately applied to this aspect of the invention.

In another aspect the invention relates to an isolated polypeptide comprising at least 70% amino acid sequence identity to (SEQ ID NO: 52).

In one embodiment the isolated polypeptide comprises at least 75%, preferably at least 80%, preferably at least 85%, preferably at least 90%, preferably at least 95%, preferably at least 99% amino acid sequence identity to SEQ ID NO: 52.

In one embodiment the isolated polypeptide comprises SEQ ID NO: 52. In one embodiment the isolated polypeptide consists or consists essentially of SEQ ID NO: 52.

In one embodiment the polynucleotide further encodes a His-tag. In one embodiment the His tag is at the N-terminus or the C-terminus of the polypeptide.

In one embodiment the polypeptide is comprised in a Colletid bee nesting material. In one embodiment the Colletid bee is a Hylaeus spp. bee. In one embodiment the Colletid bee is Hylaeus nubilosus. In one embodiment the polypeptide or portion thereof is hygroscopic or hydrophilic. In one embodiment the polypeptide or portion thereof is hydrophobic. In one embodiment the polypeptide or portion thereof is amphipathic.

In one embodiment the polypeptide is comprised in a proteinaceous polymer. In one embodiment the polymer comprises SEQ ID NO: 2 or at least a portion of SEQ ID NO: 2. In one embodiment the polymer comprises at least two copies of SEQ ID NO: 6.

In one embodiment the isolated polypeptide is encoded by a nucleic acid sequence comprising at least 75%, preferably at least 80%, preferably at least 85%, preferably at least 90%, preferably at least 95%, preferably at least 99% nucleic acid sequence identity to SEQ ID NO: 51. In one embodiment the isolated polynucleotide comprises, consists or consists essentially of SEQ ID NO: 51.

In one embodiment the polynucleotide further encodes a His-tag. In one embodiment the His tag is at the N-terminus or the C-terminus of the polypeptide.

In one embodiment of any and/or all of the above polypeptide aspects, the polypeptide is a FUN polypeptide or functional portion, analogue or derivative thereof.

In one embodiment of any and/or all of the above polypeptide aspects, the polypeptide is a recombinant FUN polypeptide or functional portion, analogue or derivative thereof,

In another aspect the invention relates to a vector that encodes an isolated polypeptide according to the invention.

In another aspect the invention relates to a vector comprising an isolated polynucleotide according to the invention.

In one embodiment the vector is selected from the group consisting of plasmids, BACs, (PACs), YACs, bacteriophage, phagemids, and cosmids. In one embodiment the vector is a plasmid.

In one embodiment the vector is selected from the group consisting of plasmids, BACs, PACs, YACs, bacteriophage, phagemids, and cosmids. Preferably the vector is a plasmid. In one embodiment the vector is an expression vector. In one embodiment the vector is PET or pFastBac.

Examples of suitable expression vectors include, but are not limited to, plasmid DNA vectors, viral DNA vectors (such as adenovirus and adeno-associated virus), or viral RNA vectors (such as retroviral vectors). In some embodiments the plasmid and/or phage vectors may be selected from the following vectors or variants thereof including pET, pFastBac, pUC18, pU19, Mp18, Mp19, ColE1, PCR1 and pKRC; lambda gt10 and M13 plasmids such as pBR322, pACYC184, pT127, RP4, p1] 101, SV40 and BPV. Additional non-limiting examples of vectors include cosmids, YACS, BACs, shuttle vectors such as pSA3, and PAT28 transposons.

Suitable viral vectors include but are not limited to vectors derived from adenovirus (AV); adeno-associated virus (AAV); retroviruses (e.g., lentiviruses (LV), Rhabdoviruses, murine leukaemia virus); herpes virus, and the like, Viral vectors employed herein can be appropriately modified by pseudotyping with envelope proteins or other surface antigens from other viruses, or by substituting different viral capsid proteins, as known, and used in the art.

The vector can be constructed to drive the expression of a polypeptide as described herein, either in vitro or in vivo. In one embodiment, the vector comprises a polynucleotide of the invention operatively linked to 5′ or 3′ untranslated regulatory sequences. The design of a vector will depend on various factors including the host cells in which the operatively linked polynucleotide is to be expressed and the desired level of polynucleotide expression.

Likewise, the selection of various promoters, enhancers and/or other genetic elements for the vector will depend on various factors including the host cells and expression levels discussed above. In one embodiment, the vector comprises a homologous promoter operatively linked to a polynucleotide of the invention. In another embodiment, the vector comprises a heterologous promoter operatively linked to a polynucleotide of the invention. In one embodiment, the homologous or heterologous promoter is an inducible, repressible, or regulatable promoter. A suitable promoter may be chosen and used under the appropriate conditions to direct high-level expression of a polynucleotide of the invention. Many such elements are described in the literature and are available through commercial suppliers.

By way of example only, promoters useful in the vector can be any suitable eukaryotic or prokaryotic promoter. In one embodiment, the eukaryotic promoter can be a eukaryotic RNA polymerase I (pol I), RNA polymerase II (pol II), or RNA polymerase III (pol III).

Expression levels of an operably linked polynucleotide in a particular cell type will be determined by the nearby presence (or absence) of specific gene regulatory sequences (e.g., enhancers, silencers and the like). Any suitable promoter/enhancer combination (see: Eukaryotic Promoter Data Base EPDB) can be used to drive expression of a polynucleotide of the invention,

Additional promoters useful in expression cassettes include B-lactamase, alkaline phosphatase, tryptophan, and tac promoter systems which are all well-known in the art. Yeast promoters include 3-phosphoglycerate kinase, enolase, hexokinase, pyruvate decarboxylase, glucokinase, and glyceraldehyde-3-phosphate dehydrogenase but are not limited thereto.

Prokaryotic promoters useful in expression cassettes include constitutive promoters as known in the art (such as the int promoter of bacteriophage lamda and the bla promoter of the beta-lactamase gene sequence of pBR322) and regulatable promoters (such as lacZ, recA and gal). A ribosome binding site upstream of the CDS may also be required for expression.

Enhancers useful in a vector as described herein include SV40 enhancer, cytomegalovirus early promoter enhancer, globin, albumin, insulin and the like.

In one embodiment, a vector may be driven by a T3, T7 or SP6 cytoplasmic expression system.

In another aspect the invention relates to an isolated host cell comprising an isolated polypeptide, isolated polynucleotide, and/or vector according to the invention.

In one embodiment the isolated host cell is a prokaryotic or eukaryotic cell.

In one embodiment the prokaryotic cell is selected from the group consisting of strains of Escherichia coli (E. coli), Pseudomonas, Bacillus, Serratia, Klebsiella, Streptomyces, Listeria, Salmonella and Mycobacteria, but are not limited thereto.

In one embodiment the eukaryotic cell is an animal cell, a plant cell, a fungal cell, or a protist cell.

In one embodiment the eukaryotic cell is a fungal cell. In one embodiment the fungal cell is a yeast cell. In one embodiment the yeast cell is a Pichia pastoris or Saccharomyces spp cell. In one embodiment the fungal cell is an Aspergillus spp. cell. In one embodiment the Aspergillus spp is Aspergillus niger.

In one embodiment the animal cell is an insect cell or a mammalian cell. In one embodiment the animal cell is a non-human animal cell. In one embodiment the mammalian cell is a non-human mammalian cell.

In one embodiment the insect cell comprises a polynucleotide as described herein in a viral vector, preferably a baculovirus. In one embodiment the insect cell is an Sf9 or High Five cell.

In another aspect the invention relates to a proteinaceous polymer comprising a FUN polypeptide or portion thereof as described herein, wherein the proteinaceous polymer comprises at least one quasi-repeat domain.

In one embodiment the proteinaceous polymer comprises from two to about 10, 20, 30, 50, 50, 60, 70, 80, 90, 100, 200, 300, 400, 500, 600, 700 800, 900, and about 1000 copies of the quasi-repeat domain.

In one embodiment the quasi-repeat domain is comprised in a FUN polypeptide or portion thereof as described herein.

In one embodiment the proteinaceous polymer comprises a glutamine composition of >25%, 26%, 27%, 28%, 29%, preferably >30% of the amino acid sequence of the polymer, a serine composition of >25%, 26%, 27%, 28%, 29%, preferably >30% of the amino acid sequence of the polymer, and a glutamate composition of >2%, 3%, 4%, preferably >5% of the amino acid sequence of the polymer.

In one embodiment the proteinaceous polymer comprises a glutamine composition of >30% of the amino acid sequence of the polymer, a serine composition of >30% of the amino acid sequence of the polymer, and a glutamate composition of >5% of the amino acid sequence of the polymer.

In one embodiment the proteinaceous polymer comprises a glutamine composition of about 30% of the amino acid sequence of the polymer, a serine composition of about 30% of the amino acid sequence of the polymer, and a glutamate composition of about 5% of the amino acid sequence of the polymer.

In another aspect the invention relates to a composition comprising an isolated polypeptide, isolated polynucleotide, proteinaceous polymer and/or vector as described herein, and a carrier, diluent, or excipient.

In one embodiment, the composition consists essentially of the isolated polypeptide, isolated polynucleotide, proteinaceous polymer and/or vector as described herein.

In one embodiment the composition is a cosmetic composition. In one embodiment the cosmetic composition is a hair or skin care composition.

In another aspect the invention relates to a method of making an isolated FUN polypeptide or portion thereof selected from the group consisting of SEQ ID NO: 2, 4, 6, 8, 10, 12, 14, 16, 18, 20, 22, 24, 50 and 52, the method comprising heterologously expressing the FUN polypeptide in an isolated host cell, and optionally purifying the FUN polypeptide.

In one embodiment expression is from a FUN polynucleotide or portion thereof selected from the group consisting of SEQ ID NO: 1, 3, 5, 7, 9, 11, 13, 15, 17, 19, 21, 22, 23, 25, 49 and 51.

In one embodiment the polynucleotide further encodes a His-tag. In one embodiment the His tag is at the N-terminus or the C-terminus of the polypeptide.

In one embodiment the FUN polynucleotide or portion thereof comprises at least one heterologous regulatory element. In one embodiment the FUN polynucleotide or portion thereof encodes a signal sequence that encodes a signal peptide. In one embodiment the signal sequences is a homologous sequence. In one embodiment the signal sequence is a non-homologous sequence.

In one embodiment the signal peptide directs the secretion of the FUN polypeptide or portion thereof.

In one embodiment the method comprises optionally purifying the FUN polypeptide after it is secreted from the isolated host cell.

In one embodiment the method comprises optionally purifying the FUN polypeptide from the isolated host cell.

In one embodiment the isolated host cell is a fungal cell, a bacterial cell or an insect cell. In one embodiment the FUN polypeptide or portion thereof is expressed from a FUN polynucleotide selected from the group consisting of SEQ ID NO: 1, 3, 5, 7, 9, 11, 13, 15, 17, 19, 21, 22, 23, 25, 49 and 51.

In one embodiment the FUN polynucleotide is comprised in a vector, preferably a baculovirus vector.

In one embodiment the polynucleotide further encodes a His-tag. In one embodiment the His tag is at the N-terminus or the C-terminus of the polypeptide.

In one embodiment the isolated host cell is a bacterial cell. In one embodiment the bacterial cell is E. coli.

In one embodiment the isolated host cell is a fungal cell. In one embodiment the fungal cell is an Aspergillus niger cell.

In another aspect the invention relates to an isolated FUN polypeptide selected from the group consisting of SEQ ID NO: 2, 4, 6, 8, 10, 12, 14, 16, 18, 20, 22, 24, 50 and 52, made by a method of the invention.

In another aspect the invention relates to a method of making a proteinaceous polymer comprising a FUN polypeptide or portion thereof selected from the group consisting of SEQ ID NO: 2, 4, 6, 8, 10, 12, 14, 16, 18, 20, 22, 24, 50 and 52, the method comprising heterologously expressing the FUN polypeptide or portion thereof in an isolated host cell under conditions that result in the expression of the FUN polypeptide or portion thereof, and optionally purifying the polymer from the host cell.

In one embodiment the proteinaceous polymer is an isolated proteinaceous polymer.

In one embodiment the FUN polynucleotide or portion thereof is expressed from a polynucleotide sequence comprising at least one heterologous regulatory element.

In one embodiment the FUN polynucleotide or portion thereof is expressed from a polynucleotide sequence comprising at least one signal sequence.

In one embodiment the signal sequence encodes a signal peptide or portion thereof. In one embodiment the signal peptide or portion thereof directs the secretion of the proteinaceous polymer from the isolated host cell.

In one embodiment the method comprises purifying the polymer after it is secreted from the host cell.

In one embodiment the method comprises purifying the polymer from the isolated host cell.

In one embodiment the isolated host cell is an isolated host cell as contemplated in the previously described aspects and embodiments of the invention.

In one embodiment the polymer comprises at least two copies of the FUN polypeptide.

In one embodiment the isolated host cell is a fungal cell, a bacterial cell or an insect cell. In one embodiment the FUN polypeptide or portion thereof is expressed from a FUN polynucleotide selected from the group consisting of SEQ ID NO: 1, 3, 5, 7, 9, 11, 13, 15, 17, 19, 21, 22, 23, 25, 49 and 51.

In one embodiment the FUN polynucleotide is comprised in a vector, preferably a baculovirus vector.

In one embodiment the isolated host cell is a bacterial cell. In one embodiment the bacterial cell is E. coli.

In one embodiment the isolated host cell is a fungal cell. In one embodiment the fungal cell is an Aspergillus niger cell.

In another aspect the invention relates to the use of a FUN polypeptide or portion thereof as described herein to coat or to form a coating on an article of manufacture. In one embodiment the coating is hydrophilic, hydrophobic or amphipathic. In one embodiment the coating is hydrophobic. In one embodiment the coating is hydrophilic. In one embodiment the coating is amphipathic.

In another aspect the invention relates to the use of a FUN polypeptide or portion thereof as described herein to make a film on an article of manufacture.

In one embodiment the film is hydrophilic, hydrophobic, or amphipathic. In one embodiment the film is hydrophobic. In one embodiment the film is hydrophilic. In one embodiment the coating is amphipathic.

In one embodiment the article of manufacture is selected from the group consisting of textiles or textile components or parts thereof and biomedical devices or components or parts thereof.

In one embodiment the article of manufacture or component or part thereof is a synthetic fiber. In one embodiment the component or part thereof is a synthetic polymer. In one embodiment the synthetic fiber or synthetic polymer is selected from the group consisting of polyester, spandex, rayon, nylon, acrylic, microfiber, neoprene, polyamide, acetate, polyvinyl chloride (PVC) and synthetic or “faux” leather or fur fibers and polymers.

In one embodiment the synthetic fiber is nylon.

In one embodiment the article of manufacture or component or part thereof is a natural fiber. In one embodiment the natural fiber is selected from the group consisting of cotton, wool, silk, coir, alpaca, flax, hemp, bamboo, sisal, and jute.

In one embodiment the natural fiber is silk.

In one embodiment article of manufacture is a textile, preferably a natural textile or a synthetic textile.

In one embodiment the article of manufacture or component or part thereof is or is comprised in or on a biomedical device. In one embodiment the biomedical device is an implantable biomedical device. In one embodiment the implantable biomedical device is selected from the group consisting of cardiovascular devices including cardioverter defibrillators, pacemakers and left ventricular assist devices, breast implants, cochlear implants, intraocular lenses, joint replacements including hip implants, catheters, dialysis tubing, contraceptive intrauterine devices, stents, sutures, staples, bandages, and wound dressings.

In one embodiment the article of manufacture is an air filtration device, component, or part thereof. In one embodiment the component or part thereof is an air filter. In one embodiment the component or part thereof is a synthetic or natural fiber and/or polymer. In one embodiment the synthetic or natural fiber and/or polymer is comprised in the air filter. In one embodiment the synthetic or natural fiber and/or polymer is in the form of a nanofiber.

Specifically contemplated as embodiments within the method and use aspects of this invention are various embodiments set out above with regards to the choice of the FUN polypeptide and portions thereof, polynucleotides encoding FUN polypeptides or portions thereof, appropriate polynucleotide regulatory sequences including signal sequences, host cells, and/or vectors that allow the expression and purification of a FUN polypeptide or portion thereof as described herein.

The invention will now be illustrated in a non-limiting way by reference to the following examples.

### EXAMPLES

### Example 1

Sourcing, Collection, and Transport of Hylaeus nubilosus Bees and their Nesting Material

Hylaeus nubilosus were collected in Queensland, Australia. A total of 13 Hylaeus nubilosus specimens were collected. The Dufour's, venom and salivary glands, and whole heads, were dissected from all 13 specimens and then either stored in DCM or ethanol.

Nest material from Hylaeus nubilosus was collected on the inside of vacated (after the larvae hatch and leave the nest) paper nesting straws (~10-20 cm length, ~0.525 cm internal diameter). Bees emerging from nesting straws were confirmed as H. nubilosus by visual observation by a trained entomologist. After emergence and identification, straws were frozen for at least 48 h, and stored frozen until required. Nest material was collected from the inside of the straws with scalpel and tweezers, and any debris was carefully removed.

### Example 2

### Identification of the Gene Encoding the Bee Nest Material Protein Using Multiple Sequencing Approaches

### Transcriptome: RNA Extraction and Sequencing

Hylaeus samples were immobilised at −20° C. for 3 minutes. Hylaeus sample dissection was performed as per the following: the heads of Hylaeus samples were cut off and transferred into RNAlater, these samples were used as an indication of transcripts enriched in the mandibular grand. The salivary and Dufour's glands were dissected into RNAlater. These samples were stored at −80° C. after dissection. RNA was extracted from the Dufour's, mandibular and salivary gland pooled samples set up triplicate for the Dufour's and Mandibular glands (2 sets of 10 and 1 set of 11 individual bees) and replicates for the salivary glands (1 sets of 10 and 1 set of 11 individual bees). The tissue was homogenised using a Tissulyser II. RNA was extracted from each of the pooled samples using the RNeasy mini kit, Sequencing was performed using an Illumina NextSeq sequencer producing 150 bp paired end reads. The RNA sequencing produced a total of 451,975,167 paired end reads (136.5 Gb of total data),

### Proteomics: Mass Spectroscopy Sequencing of the Wild Hylaeus Nest Material

Mass spectroscopy analysis was performed on the washed and enzymatically digested nesting material to identify the proteins present. The nesting material was initially washed with water, diluted organic solvent and an 8M urea solution before digesting the proteins with trypsin/chymotrypsin to generate a fingerprint of the peptide fragments. All samples were analysed by liquid chromatography coupled tandem mass spectrometry using an LTQ-Orbitrap as well as a 5600+TripleTOF mass spectrometer. Data analysis was performed using the inhouse Mascot server and the ProteomeDiscoverer 2.4 software package.

Mass spectra were searched against both transcript nucleotide sequences and predicted amino acid sequences. Both searches identified several high confidence matches to glutamine- and serine-rich peptide sequences. A BLAST search revealed that glutamine-rich sequences did not have homology to any other known protein sequences in the NCBI database (see Sequence Table 4).

Genomics: GDNA & RNA Extraction, cDNA Generation and Sequencing

Genomic DNA extraction & sequencing: The bees were flash frozen using liquid nitrogen and stored at −80° C. DNA was extracted using the following protocol. The lysis buffer was prepared by adding 1.5 μL of RNase A (100 mg/mL) to 1438.5 μL of the G2 Buffer. The bee tissue was disrupted by crushing with a DNAse-free pestle. The lysis buffer was added to the pulverised tissue. The samples were incubated at 37° C. for 30 minutes while nutating the tube. 60 μL of 20 mg/ml Proteinase K was added to the sample tube. The sample was then incubated at 50° C. for 2 hours while nutating. The sample tube was then centrifuged at max speed (12,000 g). The supernatant was subsequently transferred into a 15 ml Falcon tube. The sample was then diluted using the G2 buffer increasing the total volume to 3 ml. The QIAGEN Genomic-tip 20/G was equilibrated using 1 ml of the QTB buffer. The lysate from the falcon tube was then applied to the Genomic-tip allowing the Genomic-tip to drain, any additional remaining lysate was then added to the genomic tip. The QIAGEN Genomic-tip was then washed with 1 ml of the QC Buffer 4 times. The DNA in QC buffer was then aliquoted into three DNA Lobind 1.5 ml tubes. A 666 μL of QF buffer was added to each of the tubes. Then, 666 μL of SPRI reagent and 3 μL of SPRIselect beads were added to each of the three tubes (making sure to bring the beads up to room temperature before use and vortex immediately before use), The samples were then nutated for 10 minutes on a rocker at room temperature. The tubes were then placed on a magnet till the liquid cleared and the beads were clustered into a pellet. The supernatant was then discarded. The beads were then washed with 70% ethanol and incubated at room temperature for 30 seconds. The wash steps were then repeated. The beads were then air dried at room temperature for 30s. The pellet was then eluted using 42 μL of DNAse-free water at 50° C. by gently flicking the tube. The beads were then collected using a magnet. The samples were then pipetted into a fresh DNA Lobind 1.5 ml tube. The gDNA concentration was then measured using a Nanodrop. The size profile of the gDNA extraction was validated by running 100 ng on a 1% agarose gel and measured against a high MW DNA marker. The samples were then flash-frozen in liquid nitrogen and then stored at −80° C. DNA extraction produced 3 μg of HMW DNA. The gDNA sequencing was performed using an Oxford nanopore promethION sequencer. Subsequent analysis was undertaken using the fastq formatted sequences, which passed the default quality filtering cutoffs.

RNA extraction, cDNA generation and sequencing: The bees were flash-frozen using liquid nitrogen and stored at −80° C. RNA was extracted from 1 whole female Hylaeus using a Trizol based extraction method. First, the cuticle was removed from the bee from which RNA was extracted, and the remaining tissue was transferred into a DNA Lobind 1.5 ml tube. 50 μL of TRIzol reagent was transferred into the tube containing the samples. The tissue was then disrupted by crushing with a DNAse-free pestle. An additional 200 μl of TRIzol reagent was used to rinse the pestle, washing the remaining tissue into the tube. The sample was then incubated at room temperature for 2 minutes. A 50 μL of chloroform was added to the tube containing the sample. The tube containing the sample was vigorously shaken for 15 seconds and subsequently incubated at room temperature for 5 minutes. The sample tube was then centrifuged at 12000 g for 15 minutes at 4° C. The upper aqueous phase, containing the RNA, was transferred to a new DNA Lobind 1.5 ml tube. 0.125 ml of isopropanol was then added to the tube containing the aqueous phase. The sample was mixed through inversion, which resulted in the precipitation of the RNA. The sample was then incubated at room temperature for 2 minutes. The sample was subsequently centrifuged at 12,000 g for 10 minutes at 4° C. The supernatant was then removed and discarded. The sample was then washed with 250 μL of 75% ethanol. The sample was centrifuged at 7,500 g for 5 minutes at 4° C. The supernatant was then removed and discarded. The sample was then air dried till the RNA pellet until all droplets had evaporated. The RNA pellet was then gently resuspended in 32 μL of RNAse-free water by flicking the tube. The RNA concentration was assessed by analysing the A 260/280 and A 260/230 ratios on a Nanodrop. The sample was then flash frozen in liquid nitrogen and stored at −80° C. till. The cDNA was produced from the RNA sample using reverse transcription after poly A RNA sequence enrichment. The cDNA read data was base called using Version 4 of Guppy generating fastq sequence files. Subsequent analysis was undertaken using the fastq formatted sequences, which passed the default quality filtering cutoffs.

Assembly of the Genome from the gDNA Sequencing

Genome assembly is the process through which DNA sequencing reads are pieced together to accurately represent the nucleotide sequence and structure of an organism's genome. Genome sequencing and assembly facilitate the identification of genes, including the nest material gene in Hylaeus nubilosus.

‘Porechop’ was used to trim adapters from the gDNA sequence files (Version 0.2.4; Wick et al., 2017), Porechop used the ‘--discard_middle’ parameter. Flye (version 2.8; Kolmogorov et al., 2019) was used to assemble the genome of Hylaeus. The ‘--nano-raw’ parameter was used in the assembly in concordance with the input data being Oxford nanopore sequence. Flye was executed with the predicted ‘genome-size’ parameter of 250M. This size was used as other Hymenoptera have genomes of ~250 MB in size. The Hylaeus nubilosus genome is 360 MB in size which is surprisingly large for a bee. Genome assembly statistics imply that the assembly is highly contiguous and is therefore an accurate representation of the genome of Hylaeus nubilosus. The genome assembly is composed of 2259 scaffolds which are comparatively contiguous. The Hylaeus nubilosus assembly had an N50 of 7 and L50 of 17978421 bp, indicative of a contiguous assembly. Busco (version 5.4.2; Simão et al., 2015) analysis of the assembly using the Hymenoptera database (hymenoptera_odb10) produced a busco score of 95.7% which strongly implies the genome is assembled to a high level of completeness.

TABLE 1
Genome Assembly Statistics

| | |
|---|---|
| Genome size (MB) | 360.631 |
| Number of scaffolds | 2259 |
| Scaffold N50 | 7 |
| Scaffold L50 (bp) | 17978421 |

### Annotation of the Hylaeus Genome

The Hylaeus genome was assembled using sequence data compiled from the RNA sequencing, genomic DNA sequencing and cDNA sequencing data sets, and proteomic analysis (peptide fragment sequencing). The ‘funannotate’ (Version 1.7.4; Palmer & Stajich, 2022) genome annotation software was used which leverages the ab-initio gene predictors Augustus and GeneMark. The genome was prepared for annotation by masking uninformative regions of the genome using the ‘funannotate mask’ scripts (Palmer & Stajich, 2022). The ‘funannotate mask’ script was executed using default parameters which uses the program ‘tantan’ to perform soft masking (Frith, 2011). All RNA sequence reads, including those from the salivary, mandibular and Dufour's gland, were combined into forwards and reverse sets and used in the training of ‘funannotate.’ Porechop was used to prepare the cDNA reads for annotation by trimming of adapters (Version 0.2.4; Wick et al., 2017). Porechop was executed using the ‘--discard_middle’ parameter. The processed cDNA was used as an input for funannoate annotation training.

‘Funannotate train’ is a wrapper for transcriptome assembly using trinity (Palmer & Stajich, 2022; Grabherr et al., 2011). The ‘funannotate train’ script used the masked genome assembly, nanopore cDNA and forward and reverse RNA sequence data as inputs. The ‘funannotate train’ script was executed using the ‘-stranded’ flag set to ‘RF’ (reverse-fowards). The max intron length was set to 10000 using the ‘--max_intronlen’ parameter. ‘Funannotate train’ generated a file containing transcripts in gff3 format which are used as inputs for ‘Funannoate predict’. ‘Funannotate predict’ trains and executes Augustus and GeneMark to produce gene predictions (Version 1.7.4; Palmer & Stajich, 2022).

‘Funannotate predict’ then uses Evidence Modeller to generate consensus gene models from the Augustus and GeneMark gene predictions. The masked Hylaeus nubilosus genome assembly, the transcripts gff3 generated by ‘funannotate train’ and the ‘fundb_20200227’ (generated by ‘Funanotate setup’) were used as inputs for ‘funannotate predict’. ‘Funannotate predict’ was executed with ‘--augustus_species’ hn, the ‘--optimize_augustus’ flag, the ‘--busco_seed_species’ set to honeybee1, the ‘--buso_db’ set to hymenoptera, the ‘--organism’ set to other, the ‘--repeats2evm’ flag and ‘--max_intronlen’ set to 50000. ‘Funannotate predict’ generated the Hylaeus gff3, mRNA-transcripts and protein annotation files as output. ‘Funannotate update’ updates an annotation using RNA-seq data (Version 1.7.4; Palmer & Stajich, 2022). ‘Funannotate update’ was executed with the predicted mRNA-transcripts as the input. ‘Funannotate annotate’ is used for functional annotation of genes.

‘Funannotate annotate’ functionally annotated the Hylaeus genome annotations using input generated by eggnog, iprscan and busco (Version 1.7.4; Palmer & Stajich, 2022; Huerta-Cepas et al., 2019; Version 5.44-79.0; Jones et al., 2014; Version 5.4.2; Simão et al., 2015). Eggnog used the Hylaeus protein database and a ‘eggnog_proteins’ diamond database. The eggnog ‘-m diamond’ parameter was used to set diamond as the search method. Iprscan (interproscan) is used to provide functional annotations (Jones et al., 2014). Iprscan was executed using the default parameters besides precalculation being disabled (‘-dp’ flag) to generate functional annotations, ‘Funannotate’ predicted 75,295 protein coding genes. Busco (Version 5.4.2; Simão et al., 2015) analysis of the predicted proteins using the Hymenoptera database (hymenoptera_odb10) produced a busco score of 72.4% (15.8% fragmented).

### Identification of the Gene Encoding the Bee Nest Material Protein

The nest material gene from Hylaeus was identified using a hidden Markov model constructed using the mass spec peptide sequence data from the Hylaeus nest material. The peptide sequences from the mass spec sequence data were aligned using Muscle, and a hidden Markov model was generated using hmmbuild from the ‘Hmmer’ software suite (Version v3.8.1551; Edgar, 2004; Version 3.3.2; Johnson et al., 2010). Hmmsearch was used to search the Hylaeus trinity transcriptome protein database with the nest material gene as the highest-scoring match. Salmon and Deseq2 were used to identify high expression of our gene of interest; the gene encoding the bee nest material protein was highly expressed in the salivary glands, making up ~10% of the expressed genes in this tissue.

### Results

Using the combination of transcriptomic, genomic, and proteomic sequencing has enabled the identification of a highly unique nest material gene. This gene has an uncharacteristic gene structure comprising 14 exons and unstructured introns, is highly repetitive, and has an extraordinarily high proportion of glutamine (30.5%) & serine (32.1%) content.

The longest uninterrupted sequence assembled from peptide identifications assigned to the same sequence database entry is 86 amino acids long:

(SEQ ID NO: 33)
...SSSGMSSQAQSQQQQAQLQQSQDQRQEQESQSAGSESAVKSSKSSS
GMSSQAQSQQQQAQLQQSQAQSQEEESESAQSESEVHVSK....

A search against cDNA sequences (mRNA transcripts) of Hylaeus nubilosus revealed various highly significant hits to glutamine- and serine-rich sequences and some other protein sequences.

A BLAST search against all protein sequences in nrNCBI did not show significant homology of the glutamine-rich sequences to any other known protein sequence.

Based on the findings above and as described elsewhere herein, the inventors believe they have identified a novel nest material polypeptide from H. nubilosus. The novel polypeptide comprises 5451 amino acid residues, has a predicted molecular weight of 578.4 kDa, a glutamine (Q) and serine(S) residue content of 30.5 and 32.1%, respectively,

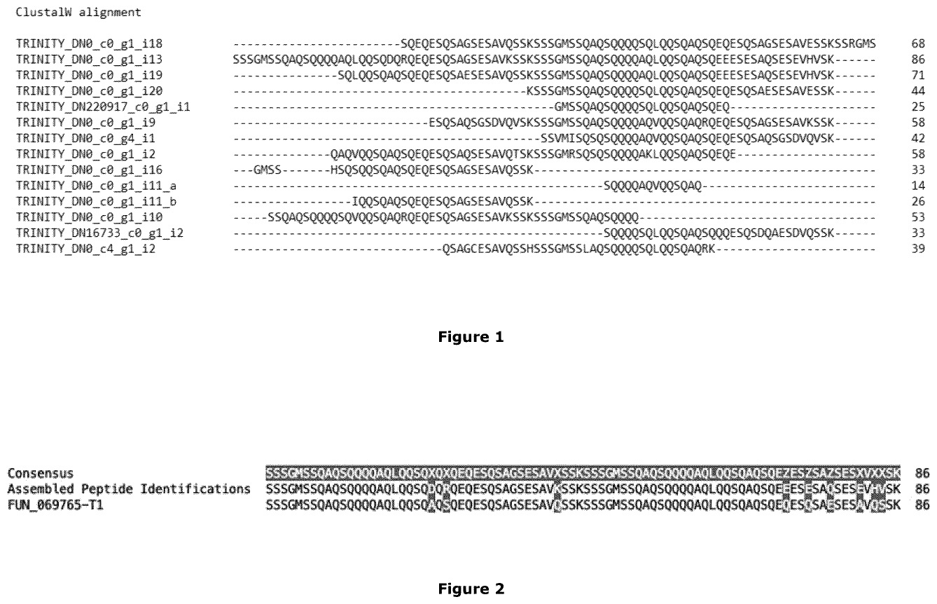

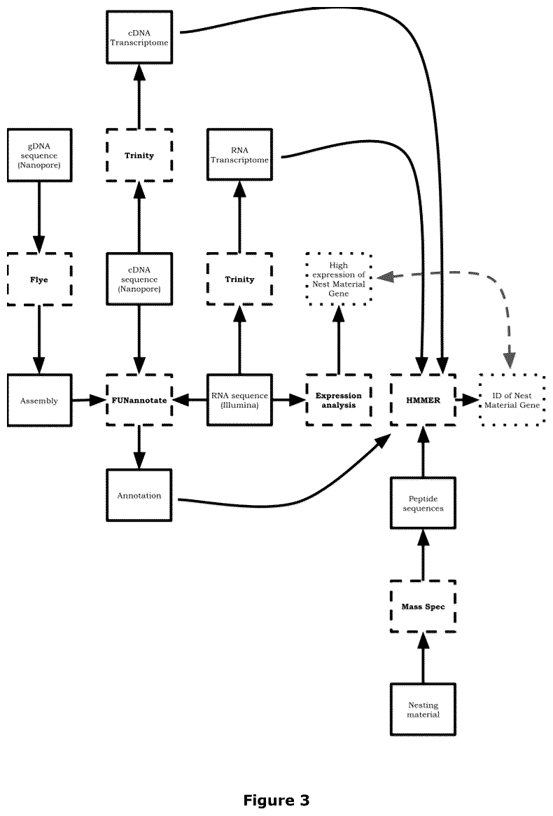

Alignment of the FUN Module 7 sequence (from the long-read sequence), SEQ ID NO 14, with the assembled proteomic sequences is shown in FIG. 1, with a promising alignment (note proteomic sequencing is challenging with lower fidelity than gene sequencing).

Mapping the predicted amino acid sequence of the long-read FUN_069765 sequence to the assembled proteomic sequence generated from the mass spec of the nest material=85%

Amino acid composition and sequence identified in proteome analysis,

SEQ ID NO: 25 is the genomic sequence (20,870 bp) for a putative H. nubilosus gene that encodes a nest material polypeptide (designated FUN_069765-T1 aka “FUN polypeptide”). Without wishing to be bound by theory the inventors believe that the FUN polypeptide forms a protein biopolymer component of the nesting material of H. nubilosus. The genomic sequence has annotated exons, introns, and untranslated regions (UTRs) and shows a predicted transcript (also designated FUN_069765-T1) and a conceptual amino acid translation.

### FUN_069765cDNA

SEQ ID NO: 1 (16,353 bp) is derived from the FUN_069765-T1 transcript sequence (I.e., after splicing of introns) and shows the FUN cDNA; i.e., only the predicted coding sequence for the FUN_069765-T1 polypeptide; it excludes any 5′ or 3′ UTRs.

SEQ ID NO: 2 is the predicted amino acid sequence of the FUN_069765-T1 polypeptide (5451 aa residues; theoretical MW=578.4 kDa) proposed to form a protein biopolymer component of the nesting material of H. nubilosus bees. SEQ ID NO: 2 is an amino acid sequence translation of the SEQ ID NO: 1, which is, in turn, derived by splicing out introns from the SEQ ID NO: 25.

### Bioinformatic Analysis of Protein Sequences

Translation of SEQ ID NO: 1 (FUN_069765cDNA) and subsequent bioinformatics analyses of the polypeptide (FUN) were performed, leading to the following observations:

The FUN polypeptide is composed of 5451 amino acids, with a theoretical molecular weight of 578,350 Da.

The majority of the protein (aa138-5451) is characterised by the presence of numerous repeated sequence motifs and, consequently, this region of the protein is designated the repeat region (see FIG. 2). The repeat region can, in turn, be further subdivided into QS-repeat regions (labelled QS in FIG. 2) that contain repeated sequences rich in glutamine (Q) and serine(S). Separating the QS-repeat regions are spacer repeats that are rich in proline (P) and cysteine (C). Details of these repeat sequences are discussed in more detail below.

In contrast, the first 137 amino acids of the protein lack any repeated sequence features. Using the SignalP 5.0 server (see Almagro Armenteros et al, 2019), the first 17 residues are predicted to be signal peptides consistent with this being a secreted protein. The predicted signal peptide is followed by an N-terminal domain (NTD or N, aa18-137) that is rich in serine (S, 16.67%), histidine (H, 15.83%), lysine (K, 11.67%) and glutamic acid (E, 10.00%).

### Predicted Amino Acid Composition of FUN 069765-T1

The full-length mature protein is rich in serine (S, 32.14%) and glutamine (Q, 30.49%) and poor in tyrosine (Y, 0.04%), phenylalanine (F, 0.07%), tryptophan (W, 0.28%), cysteine (C, 0.29%) and asparagine (N, 0.31%).

There is a distinct glutamine/asparagine bias, with glutamine occurring nearly 100 times more frequently than asparagine. Asparagine is completely absent from the QS-repeat regions,

Amino acids with large, aromatic side chains (Y, F and W) are all poorly represented and, in fact, are completely absent from the QS repeat regions, being found only in the signal peptide, N-terminal domain or spacer repeats. Similarly, cysteine and proline residues are also absent from the QS repeat regions, being found only in the signal peptide, N-terminal domain, or spacer repeats.

Sixteen cysteine residues are present. One potential N-linked glycosylation site (Asn 109-His-Thr) is present, located within the N-terminal domain.

### Example 3—Recombinant Expression and Purification of a FUN Polypeptide

The initial goal was to express a synthetic protein the properties of which could be compared to that of the nest material, To this purpose, the inventors selected the insect cell line Sf9 (derived from the fall armyworm Spodoptera frugiperda) for the expression since insects belong to the same phylum as bees. Secretory synthetic protein (SEQ No: 10) based on a fusion of two modules (module 1 and module 7) was designed, which should provide the basic elements of the nest material. Module 1 comprises of the native signal peptide (SEQ No: 47) that may be recognised by the Sf9 cellular machinery and be targeted for secretion into media. The N terminal histidine rich region of the native module 1 was replaced by a hexahistidine tag for affinity purification.

The cDNA of SEQ No: 10 was cloned into pFastBac Dual entry vector from Invitrogen. The plasmids are best propagated through E. coli DH5a cells or similar, Selection in either liquid LB or LB agar supplemented with 75 μg ml-1 ampicillin is preferred. Transformation of E. coli DH5α cells with entry vectors is performed by a basic chemical heat shock protocol. Recovery of propagated entry vectors was performed by a chemical alkaline lysis protocol. Competent E. coli DH10 Multibac cells (Geneva Biotech), containing bacmid (baculovirus genome vector plasmid) and a helper plasmid, were used to generate recombinant bacmids according to the manufacturer's protocol (Invitrogen). Recombinant bacmids (FUN polypeptide bacmid) from confirmed white DH10 Multibac cells are recovered from the cells by the alkaline lysis method. Insertion of the gene into the bacmid was verified by PCR.

Sf9 cells were transfected with recombinant bacmid DNA using insect genejuice transfection reagent (Merck) in 6-well plates. Sf9 cells were propagated at 28° C. in Sf900-III serum-free insect cell culture medium (Thermo fisher). Sf9 cells were grown either as monolayers on cover slips in 6 well plates or in shaker flasks agitated at 130 rpm. The cells were Incubated for 4 h at 28° C., rinsed, and incubated for another 72 hours. Following 72 hours of incubation the transfection mix was removed into a sterile falcon tube, residual Sf9 cells were pelleted by centrifugation (500 g) and the clarified media containing baculovirus taken in a fresh 6-well plate. The primary amplification plates were incubated at 28° C. for a further 72 hours. Amplified virus was harvested, cells removed by centrifugation at 500 g and the clarified viral stock supplemented with 2% fetal calf serum and stored at 4° C. For expression of FUN Polypeptide, Sf9 cells (~2.0-2.5×10⁶ cells/ml) were infected with the generated recombinant viruses.

Small scale protein expression was conducted in 200 ml volumes of Sf9 cells in serum free media. Cultures were infected with the empirically derived ratio of virus and incubated at 28° C. with shaking at 130 rpm. Cultures were routinely supplemented with sterile D-Glucose, L-Serine, and L-Glutamine to 5 mM at t48 and t72. Cultures were typically taken out to between 72-96 hours post infection and centrifuged at 1500 g before protein harvest.

FUN polypeptide expression was scaled up in 250 ml shake flasks to a total volume of 4.4 L. Media was harvested and stored frozen at −20° C. for several weeks before downstream processing. The 4.4 L of media was processed using NaCl-isopropanol method which was used for biomaterials generation (B2.0).

The 4.4 L of culture media was stored frozen at −20° C. in Schott bottles. The media was then thawed at 4° C. for 2 days for further processing. The NaCl-isopropanol fractionation was used to process the media (at pH ~5.8) as outlined below. A 500 ml media was measured in a polypropylene measuring cylinder and poured into 1 L Schott bottle. This was titrated to 2M NaCl concentration by gradual addition of salt with stirring. On complete dissolution of NaCl, a 500 ml of 100% isopropanol was added to the mixture (1M final NaCl concentration) with thorough stirring. The mixture was stirred for 30 minutes and spun at 6750 g for 45 minutes. This was repeated for the entire 4.4 L media and the spun pellets were recovered and resuspended in 1×PBS at pH 7.4 with 5% glycerol. The pellets in PBS taken in falcon tubes, were given another spin at 4000 g for 30 minutes and the supernatant was discarded (PBS wash). Pellet was mixed in a NuPAGE SDS loading buffer, boiled to 90° C. for 10 minutes, and loaded onto a precast 12% NuPAGE Bis-Tris gel (Thermo Fisher scientific). The electrophoresis was conducted at 160V for 40 minutes using 1×MOPS running buffer (Thermo Fisher scientific) and the protein bands were visualised by staining with Coomassie brilliant blue R-250.

The pellets were resuspended in PBS following which the slurry was flash-frozen in liquid nitrogen and stored at −80° C. Pellets were thawed on ice and spun at 4000 g for 30 minutes following which the supernatant was discarded. The product (wet weight ~11 g) was then shipped on cold chain.

SDS page gel analysis of NaCl-isopropanol precipitated material was used to determine that the purity of the expressed FUN polypeptide 2.0 was quite low (estimated purity of <30%) (not shown). Further purification of the FUN polypeptide was carried out as described below.

The culture media (harvested at t120) was spun at low g to pellet cells and debris. In order to reduce the working volume, NaCl-isopropanol fractionation was included as mentioned above. The pellet containing FUN polypeptide was resuspended in PBS+5% glycerol (⅕^{th} of starter culture media volume) with a minimum of 6M urea at pH=7.5 or 8. This was then taken for sonication to disperse the proteins and free up the His-tag further. Sonication was performed for a total of 1 minute (1 second on, 1 second off) at an amplitude of 10 (approximately 1600 J of energy applied). The media was then taken for centrifugation at 10,000×g for 20 minutes. The clarified supernatant was loaded onto a 5 mL NI-HisTrap FF (Fast Flow) column equilibrated with wash buffer (25 mM sodium phosphate, 300 mM NaCl, 10 mM imidazole, 5% glycerol, 6M urea). The protein was eluted from the column with an elution buffer (25 mM sodium phosphate, 300 mM NaCl, 300 mM imidazole, 5% glycerol, 6M urea), dialysed overnight, stirring, against a buffer (containing 25 mM NaP, 150 mM NaCl, and 5% glycerol at pH=7.5) to remove urea and imidazole.

### Composition

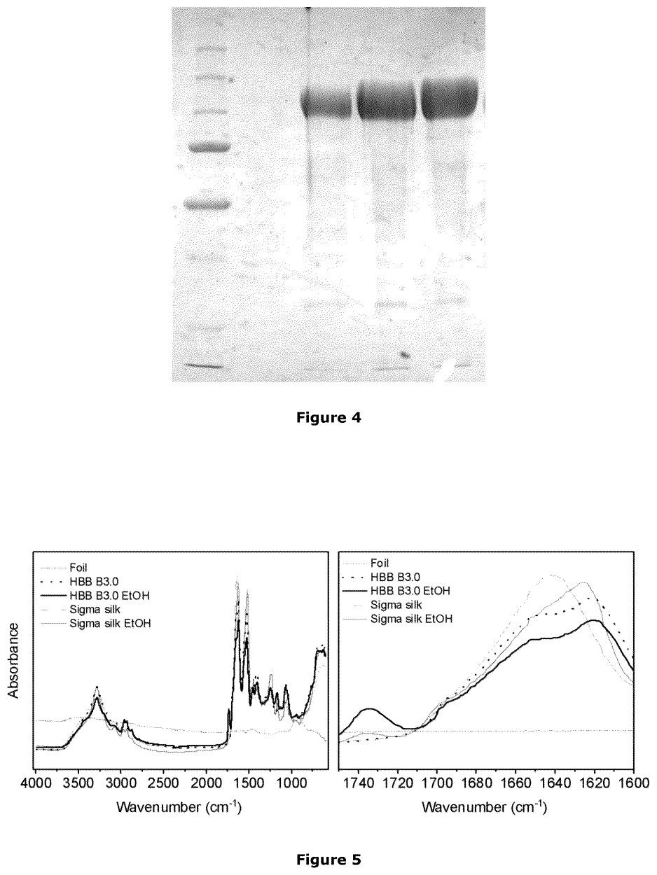

Further characterization was undertaken of the NaCl-isopropanol precipitated, centrifuged and filtered FUN polypeptide protein to elucidate the nature of this sample. Aside from monitoring the protein molecular weight using SDS-PAGE (FIG. 4), chemical characterisation was also performed using FT-IR (FIGS. 5 and 6).

### Preliminary Coating

Before attempting to spin fibers, typically coating and films are cast of the target protein, and the chemical and physical properties of the material are evaluated. Coatings were cast with FUN polypeptide material on different substrates using a drop casting method. The drop casting is a deposition method suitable for small-area film formation. The method is performed by dropping the 50-100 μL FUN polypeptide samples (~1 mg/mL) on the desired substrate and letting the solution evaporate for a desired amount of time. FUN polypeptide coatings were then characterised using a range of different techniques.

### Fourier-Transform Infrared Spectra (FTIR)

50 μL FUN polypeptide samples (~1 mg/mL) were drop-cast on 1×1 cm aluminium (FIG. 5) and glass coverslips (FIG. 6) which were subsequently mounted on a solid support (whole glass slide). The Fourier-transform infrared (FTIR) spectra of these samples were tested on a Bruker Lumos FTIR spectrophotometer with the attenuated total reflection (ATR) mode. The IR spectra were taken at an absorption at 400-3900 cm-1 at a resolution of 2 cm-1 and a total of 128 scans. Significant β-sheets formation was observed from the FTIR spectrum of a FUN polypeptide drop cast film. The presence of β-sheets would render the protein substrate insoluble in water, which is critically important for coating and fiber related applications. In comparison, the FTIR spectrum of a silk fibroin cast film showed the presence of a-helix/coil structure primarily. Upon treatment with ethanol, the α-helices turned to β-sheets (the peak shift observed in the FTIR spectrum). As shown in this work, the FUN polypeptide formed β-sheets even before any ethanol treatment. These results provide a structural characterization of the FUN polypeptide as it would be expected to behave in the H. nubilosus nesting material and demonstrate that the FUN polypeptide (and various compositions comprising the FUN polypeptide) as provided herein has significant potential for use as a functional coating and fiber forming material.

### Scanning Electron Microscopy (SEM) Characterization

SEM was employed to observe the surface morphology of the FUN protein coatings described above. For SEM analysis, FUN polypeptide (5 μl of 0.5 mg/ml) microfilm was cast on silicon wafer and analysed by scanning electron microscopy using a Supra 55-VP field emission scanning electron microscope (SEM, Zeiss, Germany) at an accelerating voltage of 3 keV. Images were captured at 7-8 mm working distance at 10,000× and 100,000×. FIG. 7 shows the typical SEM image of FUN polypeptide coating, the surface of which is smooth and clean except for the fibril or network-like patterns. The results from the SEM work are congruent with what is currently known about the surface morphology of bee nesting material, i.e., coatings and films constructed from the FUN polypeptide described herein demonstrate the characteristic patterns of bee nest material (as would be expected).

### Water Contact

Using a drop casting technique, recombinant protein films of the FUN polypeptide were cast on piranha cleaned glass slides (glass slides from Westlab, Australia) with a surface area of 1×1 cm² by dropping 100 μL concentrated protein solution (about 2 mg/ml). Water contact angle measurements were conducted to investigate the hydrophobicity of film surfaces. Using the static sessile drop technique analysed using a Biolin Attention Theta Flow Tensiometer, measurements were undertaken on FUN polypeptide and silk coatings (Sigma) as well as ethanol treated FUN polypeptide and silk coatings (Sigma). On the protein-film-cast glass substrate, water droplets were applied. Images were captured immediately upon contact and after 30s of incubation at room temperature. Surfaces with contact angle measurements below 90° are indicative of hydrophilicity. Glass substrate was hydrophilic and showed an angle of 14° that remained constant after 30 second equilibration. FUN polypeptide coated surfaces showed contact angles of around 74°, which upon 30 seconds equilibration reduced to around 62° whereas that of sigma silk showed around 48°, which upon 30 seconds equilibration reduced to around 22°. Coatings rendered glass were more hydrophobic, indicated by better spread of droplets on non-coated glass substrates to that after protein coating. Ethanol treated FUN polypeptide FUN polypeptide exhibited a hydrophobic angle of 96 degrees immediately upon contact, which after 30 seconds equilibration reduced to 75°. Therefore, it is clear from the results presented herein that the FUN polypeptide FUN polypeptide or at least a portion thereof, is relatively more hydrophobic than the sigma silk when measured under the same conditions.

### Washability

A simple test was also conducted to confirm the stability of the FUN polypeptide coatings. 50 μL FUN polypeptide samples (~1 mg/mL) were drop-cast in 1×1 cm glass slides. The slides were soaked in pure ethanol solution for 2 hrs and were then dried under vacuum until further used. Washability tests were done by soaking the FUN polypeptide coated glass slides in water and or 0.1 M PBS Buffer (pH 7.4) for 24 hours. The soaked glass slides were rinsed with deionised water to remove excess salts. The dried “soaked” slides were stained with Coomassie Brilliant Blue dye for 3-4 hrs and were de-stained in deionized water overnight. After the coating was soaked in water or PBS for 24 hours, the coating still remained intact, indicated by the full coverage of the Coomassie stain (FIG. 9),

### Example 4—Proteomics or Other Analyses that Support Conclusions from Previous Examples

### Proteomics

SDS-PAGE gels following protein purification were used to excise bands of interest for mass spectrometry fingerprinting. The highlighted boxes point to the presumptive FUN polypeptide bands. The excised FUN polypeptide bands were pooled and sent to the University of Auckland Mass Spectrometry Facility to perform the fingerprinting.

### Mass Spec Fingerprinting

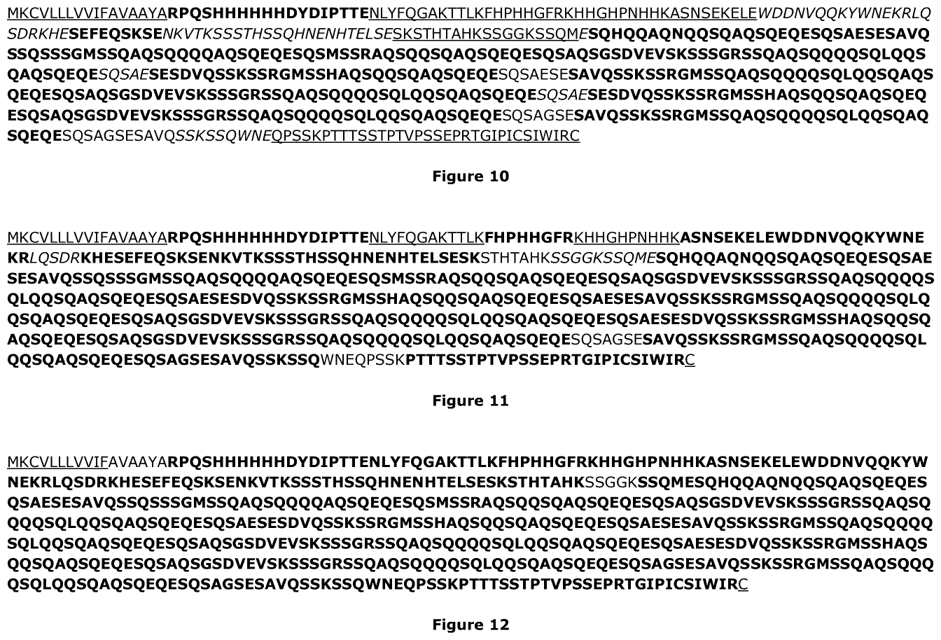

The objective is to achieve sequence coverage, ensure the bands of interest at 100 kDa on denaturing gel is the expected protein, and it is the exact sequence as designed in the construct. GluC digestion of the FUN polypeptide protein band produced a sequence coverage of ~83% (FIG. 10), with the peptides underlined depicting unobserved regions. The signalling peptide is expected to be cleaved upon secretion from the Sf9 cell. Theoretically, there is a good chance to pick up ‘SKSTHTAHKSSGGKSSQME’ peptide (SEQ ID NO: 26) in a GluC digest; however, this peptide produced no coverage. Due to the presence of two cysteines in the C-terminal, some proportion of post translational modifications are expected here, which could have led to the no-confidence match, To achieve complete sequence coverage, Trypsin digestion was employed along with reduction and alkylation, which was thought to provide better coverage at the C-terminal region (FIGS. 11 and 12).

The reduced and alkylated Trypsin digest picked up the stretch, almost covering the entire C-terminal region (FIG. 11) except for the last cysteine residue, which could be clipped off by Trypsin. The preceding stretch of the C-terminus matched to two high-quality peptides (‘PTTTSSTPTVPSSEPR’ (SEQ ID NO: 27) and ‘TGIPICSIWIR (SEQ ID NO: 28)) and one moderate scoring one to cover SSQWNEQPSSK (SEQ ID NO: 29). So, the combined Trypsin and GluC digests give ~96% coverage overall. Trypsin digest has a good chance of picking up NLYFQGAK (SEQ ID NO: 30) and KHHGHPNHHK (SEQ ID NO: 31), but a suspected glycopeptide around ‘TLKFHPHH’ (SEQ ID NO: 32) between these peptides could be hindering its cleavage.

Alkylated trypsin digestion (FIG. 12) picked up the entire stretch except the last cysteine residue, giving an overall coverage of 99.81%.

### Example 5—HnM1M7-01

### HnM1M7-01 Construct

Secretory synthetic protein (SEQ No: 10) based on the fusion of two modules (HnM1M7-01) begins with the proposed native signal peptide (SEQ No: 47), which may be recognised by the Sf9 cellular machinery and targeted for secretion into media. In addition to containing the secretory signal peptide, the construct was designed to incorporate a hexahistidine tag for affinity purification and an rTEV protease recognition motif for tag removal. This example describes the optimised protocol for the expression and purification of HnM1M7-01 in the SF9-baculovirus system.

### Generation of Recombinant Bacmid

The entry vector was used to transform competent E. coli DH10 Multibac cells (Geneva Biotech) containing bacmid (baculovirus genome vector plasmid) and a helper plasmid to generate recombinant bacmids following the procedure provided by the manufacturer (Invitrogen). Briefly, 100 ng of the entry vector (HnM1M7-01 in pFastBac-DUAL) was transformed into chemically competent DH10 MultiBac cells and plated out on KGTIX plates (kanamycin, gentamycin, tetracycline, IPTG and X-gal). The selection of successful recombinants (white colonies) was confirmed by replating a number of white colonies onto fresh KGTIX plates and ensuring a stable colour phenotype (against a control non-recombinant blue colony plated on the same selection plates). Recombinant bacmids (HnM1M7-01 bacmid) from confirmed white DH10 Multibac cells were recovered from the cells by chemical alkaline lysis and used to transfect log phase Sf9 cells.

### Sf9 Transfection & Viral Amplification

Sf9 cells were transfected with recombinant bacmid DNA in 6 well plates using the insect GeneJuice transfection reagent (Merck). Log phase Sf9 cells at ~0.8×10⁶/ml were plated into the well of a 35 mm 6 well cell culture plate (~80% confluence) and allowed to adhere to the plate at 28° C. for 60 minutes. Four micrograms of recombinant bacmid was incubated with 10 μl of transfection reagent (Merck) in a total volume of 200 μl Sf900-III media at room temperature for 30 minutes, then made up to 1 ml with fresh Sf900-III media. Media from the 6 well plates was aspirated from the adherent Sf9 cells, and 1 ml of the HnM1M7-01 bacmid/GeneJuice solution was overlaid on the cells. The cells were incubated for 4 h at 28° C., rinsed, and supplemented with an extra 1 ml of SF900-III media. Following 72 hours of incubation, the transfection mix was removed into a sterile falcon tube, residual Sf9 cells were pelleted by centrifugation (500 g), and the clarified media containing baculovirus was taken in a fresh 6-well plate. Each well was then overlaid with 1.6 ml of Sf9 cells at 1.5×10⁶ cells/ml and incubated at 28° C. for a further 72 hours. The amplified virus was harvested, cells removed by centrifugation at 500 g, and the clarified viral stock was supplemented with 2% fetal calf serum. Amplified viral stocks were stored at 4° C. prior to titration and expression. For the expression of HnM1M7-01, the amplification round was kept minimal (not more than one round) to avoid deletion events of the gene of interest.

### HnM1M7-01 Expression

For expression of HnM1M7-01, Sf9 cells at ~2.0-2.5 ×10⁶ cells/ml were infected with the generated recombinant viruses in shaker flasks. Small-scale protein expression was conducted in 200 ml volumes of Sf9 cells in serum-free media. Cultures were infected with the recombinant virus at a ratio of between 1:5 k and 1:10 k and incubated at 28° C. with shaking at 130 rpm. Cultures were routinely supplemented with sterile D-Glucose, L-Serine, and L-Glutamine to 5 mM at t48 and t72, and typically harvested at 96 hours post-infection. The media was clarified by centrifuging at 1500 g before protein harvest.

### HnM1M7-01 Purification

The clarified media was adjusted to 50 mM Tris.Cl pH 8.0 and 2M NaCl. On complete dissolution of NaCl, an equal volume of 100% isopropanol was added to the mixture (1M final NaCl concentration) with thorough stirring. The mixture was stirred for 30 minutes and spun at 6750 g for 45 minutes. The spun pellets were resuspended in sonication buffer (25 mM Tris pH 8.0, 300 mM NaCl, 6M Urea & 5% glycerol) up to ⅕th of starter media volume. Sonication was performed on ice at 75% amplitude with 1 second pulses, 1 second pause between pulses until a minimum of 80,000 J was applied (Qsonix midi tip). The solution was clarified by centrifugation at 10,000×g for 20 minutes. The clarified supernatant was adjusted to 15 mM imidazole and loaded sequentially onto a sonication buffer equilibrated 5 mL Ni-HisTrap FF (Cytiva), Following protein loading, the column was washed with 5×CV wash buffer (25 mM Tris pH 8.0, 300 mM NaCl, 6M Urea & 5% glycerol) and bound protein step eluted with elution buffer (25 mM Tris pH 8.0, 300 mM NaCl, 15 mM imidazole, 6M Urea & 5% glycerol). The eluted protein fractions were collected and analysed on a 10% PAGE Tris-glycine gel to estimate protein purity. Protein fractions were pooled and dialysed against 2 L of dialysis buffer (25 mM Tris pH 8.0, 150 mM NaCl, & 5% glycerol) at 4° C. overnight.

### Emulsification Properties of HnM1M7-01

This example aims to demonstrate the emulsion stabilising effect of the HnM1M7-01 protein. To this purpose, the inventors used HnM1M7-01 as a self-emulsifier to make oil/water emulsions without the addition of any other surface active agents.

### Method:

HnM1M7-01 solution was prepared as described in example 5.1. Oil in water (O/W) emulsions were made using MCT oil (Caprylic/capric triglyceride, New Directions Australia) as the oil phase and buffer (control) or protein solution (treated) as the aqueous phase at a final ratio of 1:1. Samples were made up by mixing aqueous protein solution and MCT oil that contained 5 μg/mL of Nile Red (Merck). The mixture was emulsified using an Omi Sonic Ruptor 400 ultrasonic homogeniser with a 3.8 mm very high-intensity processing tip, Emulsification consisted of applying 10×1 second pulses at a 50% power limit, Emulsions were imaged using a Nikon Eclipse Ti-S inverted microscope in fluorescence mode using the Texas Red filter. Samples were imaged at 1, 2, and 7 days to assess emulsion stability. The tubes were also photographed to assess bulk phase separation.

### Result:

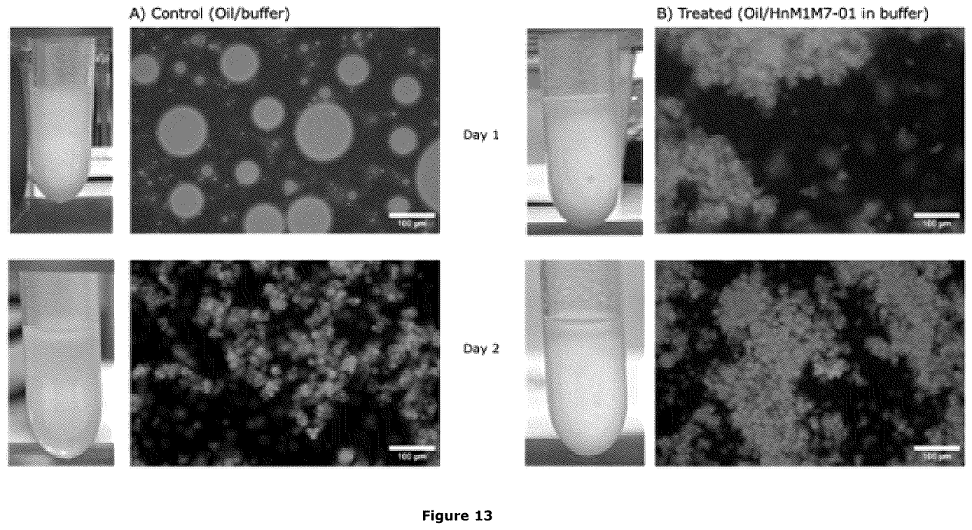

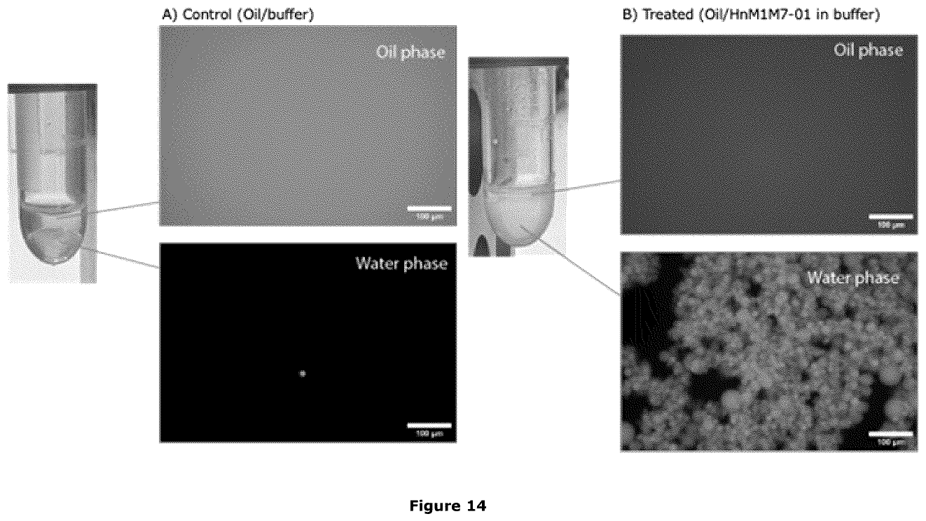

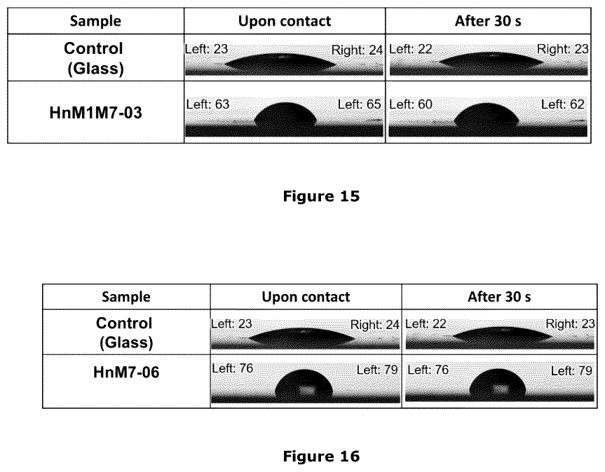

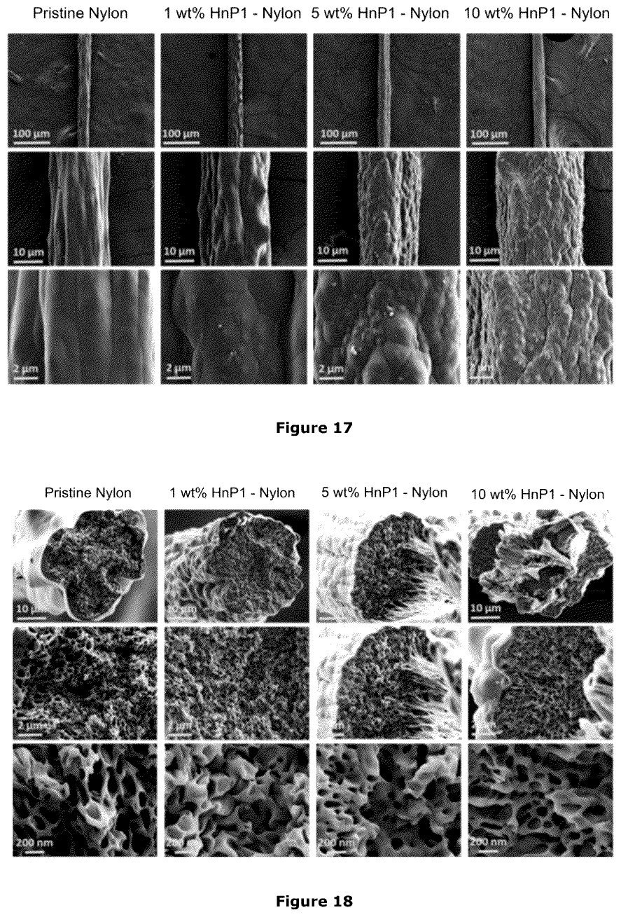

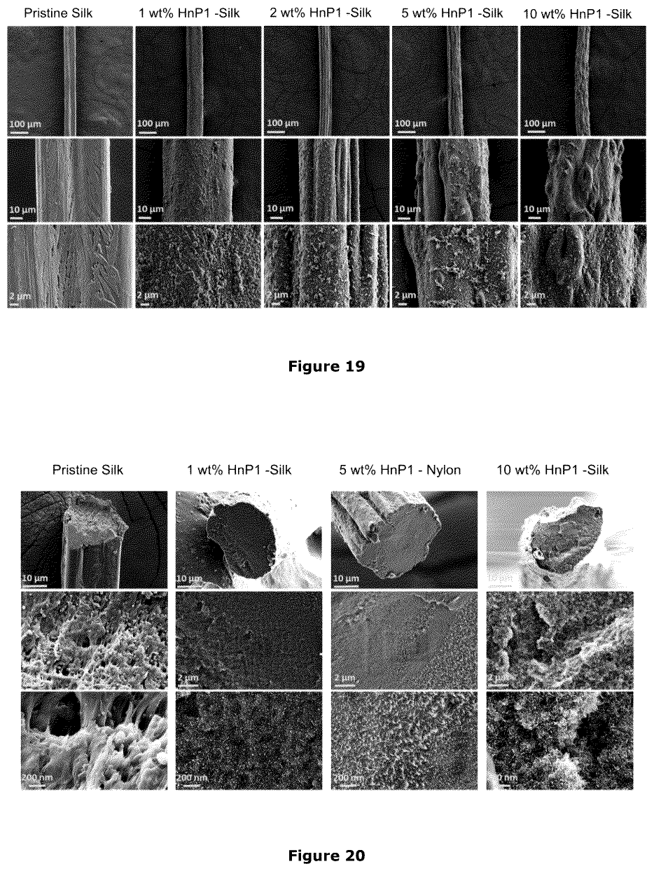

Spheres (Nile Red dissolved in oil, seen as white spheres in FIG. 13) would be seen if the buffer layer contained oil droplets, which implies an emulsion was formed. The confocal image of sample on days 1 and 2 showed the presence of spheres consisting of emulsified oil-protein solution (FIG. 13B), reflecting some surface activities exhibited by HnM1M7-01 modified mixtures. Although the buffer/oil mixture (untreated) also showed the formation of oil droplets/spheres in the aqueous phase during the first two days of the experiment (FIG. 13A, days 1 & 2), these spheres were not stable and were invisible after day 7 (FIG. 14A). Meanwhile, the spheres observed in HnM1M7-01-stabilized mixtures were found to be stable, even equilibrating for 7 days.

On the basis of these observations, the inventors demonstrated that HnM1M7-01 possesses surface activity. Without wishing to be bound by theory, the inventors believe this surface activity could be due to its inherent amphiphilic structure. Therefore, in the oil/HnM1M7-01 in the buffer system, protein molecules present at the oil/buffer interface would be adsorbed there to minimise the interface tension. HnM1M7-01 at the interface would be rearranged to expose hydrophilic chains toward the buffer phase and hydrophobic chains toward the oil phase, which consequently facilitated the stabilisation of oil/buffer emulsions in this mixture.

### Example 6—HnM1M7-03—(SEQ ID NO: 50)

### Expression and Purification of HnM1M7-03

This example describes the production of a polypeptide in E. coli BL21 (DE3) using a kanamycin resistant plasmid and the resulting production of purified polypeptide HnM1M7-03.

Nucleic acid sequences coding for synthesis of HnM1M7-03 protein were synthesized using non-template PCR. In short, virtual nucleic acid sequences were converted into oligonucleotide sequences using software suite LIMS (DNA TwoPointO. Inc., Newark, CA, USA). Full length nucleic acid sequences were synthesized by assembling oligonucleotides using template-free PCR. An amplicon was purified and cloned using standard cloning methods (Molecular Cloning. A Laboratory Manual. 2012. Green and Sambrook).

A gene coding for synthesis of HnM1M7-03 protein was cloned into expression vector pD451—SR, containing T7 inducible promoter (DNA TwoPointO. Inc., Newark, CA, USA). Purified plasmid containing the gene was transformed into chemically competent E. coli BL21 (DE3) cells via heat shock and plated on non-inducing agar with 0.1 mg/L kanamycin. Plates were incubated overnight at 37° C. Glycerol stocks were prepared by selection and growth of a single colony from a transformation plate in non-inducing media, followed by suspension of cells in media containing glycerol, and preservation by storage at −80° C.

E. coli BL21 (DE3) containing plasmid capable of expressing HnM1M7-03 protein was grown in 100 L fermenters. Media was prepared and autoclaved in the fermenter. Media component and concentrations were as follows: casein hydrolysates, 12 g/L; yeast extract, 24 g/L; NaCl, 10 g/L; K2HPO4, 8 g/L; glycerol, 30 g/L. Kanamycin, 50 mg/L was added when media had cooled. Pre-culture 1 flasks were grown at 37° C. for approximately 6 hours. Pre-culture 2 flasks were Inoculated from pre-culture 1 and grown at 28° C. for approximately 12 hours. The fermenter was inoculated from pre-culture 2 flasks and temperature controlled at 37° C. for initial growth phase. Dissolved oxygen was controlled to 30% air saturation and the fermenter was maintained at pH 6.8. Pluronic antifoam, 5 g/L was added to control foaming.

Immediately before induction the fermenter was cooled down to 20° C. and expression of HnM1M7-03 protein was induced at OD600 approximately 2 using 0.2 mM IPTG. The biomass was concentrated by tangential flow filtration (TFF) approximately 22 hours post induction and harvested by centrifugation. Biomass was frozen at −20° C. until further processing,

Biomass was thawed overnight and resuspended in lysis buffer (25 mM Tris, 2 mM MgCl₂ 0.5% (w/v) TritonX-100 pH 8.0) using a Miccra D-9 rotor-stator. Lysis was performed at room temperature for 40 minutes using 2 mg lysozyme per gram of biomass, DNA was degraded with 25 units of benzonase per gram of biomass. Insoluble material was collected by centrifugation at 17,000 g for 20 minutes. The lysate pellet (i.e., ‘insoluble’ fraction) was subjected to washing in 25 mM Tris, 2 mM MgCl₂, 0.5% (w/v) TritonX-100 pH 8.0 for 40 minutes. Insoluble material was collected by centrifugation at 17,500 g for 40 minutes. The washed pellet was subjected to further washing in 0.05M sodium phosphate pH 11.5. Insoluble material was collected by centrifugation at 17,000 g for 40 minutes. The washed pellet was subjected to extraction in 10 mM Tris, 4M guanidine pH 8.0 for 40 minutes at room temperature. The extracted fraction containing HnM1M7-06 protein was centrifuged at 17,000×g for 20 mins to remove debris, and the supernatant filtered. The extracted fraction was diluted into immobilized metal affinity chromatography (IMAC) loading conditions (10 mM Tris, 4M guanidine 500 mM NaCl, 20 mM Imidazole, pH 8.0). The diluted material was loaded onto a HiScale column packed with IMAC Sepharose 6 Fast Flow resin (Cytiva) charged with Nickel. The IMAC column was washed with loading buffer (10 mM Tris, 2M guanidine, 0.5M NaCl, 20 mM imidazole, pH8.0), and HnM7-06 protein recovered with elution buffer (10 mM tris, 2M guanidine, 0.5M NaCl, 500 mM imidazole pH8.0).

HnM1M7-03 protein was precipitated from elution fractions using 2M ammonium sulphate. Precipitated protein was recovered by centrifugation at 12,000 g for 10 minutes. Precipitated protein pellets were resuspended in ultrapure water and washed HnM1M7-03 rotein precipitate collected by centrifugation at 12,000 g for 10 minutes.

### Physical Properties of HmM1M7-03

Surface Water Contact Angle of a HnM1M7-03 Protein Coated Glass Surface Protein solutions (1 mg/mL HnM1M7-03, dissolved in 98% formic acid) were drop-cast on glass slides, followed by drying at room temperature overnight. The coated glass slides were fixed in a Theta Flow Tensiometer (Biolin Scientific, UK). The water contact angles (WCA) were recorded continuously over 30 s.

The surface of untreated glass was hydrophilic, showing a water-contact angle of 22-24° that remained constant after 30 seconds of equilibration. Glass coated with HnM1M7-03 showed contact angles of 60-65°, which was maintained after 30 seconds equilibration. The inventors hereby demonstrate that HnM1M7-03 is a useful material for modifying the relative hydrophobicity of materials, providing the ability to impart useful properties to various articles when used as a film or coating.

### Example 6—HnM7-06 (SEQ ID NO: 52)

### Expression and Purification of HnM7-06

This example describes the production of a polypeptide in E. coli BL21 (DE3) using a kanamycin resistant plasmid and the resulting production of purified polypeptide HnM7-06.

Nucleic acid sequences coding for synthesis of HnM7-06 protein were synthesized using non-template PCR. In short, virtual nucleic acid sequences were converted into oligonucleotide sequences using software suite LIMS (DNA TwoPointO. Inc., Newark, CA, USA), Full length nucleic acid sequences were synthesized by assembling oligonucleotides using template-free PCR. An amplicon was purified and cloned using standard cloning methods (Molecular Cloning, A Laboratory Manual. 2012. Green and Sambrook).

A gene coding for synthesis of HnM7-06 protein was cloned into expression vector pD451-SR, containing T7 inducible promoter (DNA TwoPointO. Inc., Newark, CA, USA). Purified plasmid containing the gene was transformed into chemically competent E. coli BL21 (DE3) cells via heat shock and plated on non-inducing agar with 0.1 mg/L kanamycin. Plates were incubated overnight at 37° C. Glycerol stocks were prepared by selection and growth of a single colony from a transformation plate in non-inducing media, followed by suspension of cells in media containing glycerol, and preservation by storage at −80° C.

E. coli BL21 (DE3) containing plasmid capable of expressing HnM7-06 protein was grown in 100 L fermenters. Media was prepared and autoclaved in the fermenter. Media component and concentrations were as follows: casein hydrolysates, 12 g/L; yeast extract, 24 g/L; NaCl, 10 g/L; K2HPO4, 8 g/L; glycerol, 30 g/L, Kanamycin, 50 mg/L was added when media had cooled. Pre-culture 1 flasks were grown at 37° C. for approximately 7 hours. Pre-culture 2 flasks were inoculated from pre-culture 1 and grown at 28° C. for 17 hours. The fermenter was inoculated from pre-culture 2 flasks and temperature controlled at 37° C. for initial growth phase. Dissolved oxygen was controlled to 30% air saturation and the fermenter was maintained at pH 6.8. Pluronic antifoam, 5 g/L was added to control foaming.

Immediately before induction the fermenter was cooled down to 20° C. and expression of HnM7-06 protein was induced at OD600 approximately 2 using 0.2 mM IPTG. The biomass was concentrated by tangential flow filtration (TFF) and harvested by centrifugation approximately 22 hours post induction. Biomass was frozen at −20° C. until further processing,

Biomass was thawed overnight and resuspended in lysis buffer (25 mM Tris, 2 mM MgCl₂ 0.5% (w/v) TritonX-100 pH 8.0) using a Miccra D-9 rotor-stator. Lysis was performed at room temperature for 40 minutes using 2 mg lysozyme per gram of biomass. DNA was degraded with 25 units of benzonase per gram of biomass. Insoluble material was collected by centrifugation at 12,000 g for 20 minutes. The lysate pellet (i.e., ‘insoluble’ fraction) was subjected to washing in 25 mM Tris, 2 mM MgCl₂, 0.5% (w/v) TritonX-100 pH 8.0 for 40 minutes. Insoluble material was collected by centrifugation at 17,500 g for 40 minutes. The washed pellet was subjected to further washing in 0.05M NaOH. Insoluble material was collected by centrifugation at 17,500 g for 20 minutes. The 0.05M NaOH washed pellet was subjected to extraction in 10 mM Tris, 4M guanidine pH 8.0 for 40 minutes at room temperature. The extracted fraction containing HnM7-06 protein was centrifuged at 17,500×g for 20 mins to remove debris, and the supernatant filtered. The extracted fraction was diluted into immobilized metal affinity chromatography (IMAC) loading conditions (10 mM Tris, 4M guanidine 500 mM NaCl, 20 mM Imidazole, pH 8.0). The diluted material was loaded onto a HiScale column packed with IMAC Sepharose 6 Fast Flow resin (Cytiva) charged with Nickel. The IMAC column was washed with loading buffer (10 mM Tris, 2M guanidine, 0.5M NaCl, 20 mM imidazole, pH8.0), and HnM7-06 protein recovered with elution buffer (10 mM tris, 2M guanidine, 0.5M NaCl, 500 mM imidazole pH8.0).

HnM7-06 protein was precipitated from elution fractions using 2.5M ammonium sulphate. Precipitated protein was recovered by centrifugation at 12,000 g for 10 minutes. Precipitated protein pellets were resuspended in ultrapure water and washed HnM7-06 protein precipitate collected by centrifugation at 12,000 g for 10 minutes.

### Physical Properties of HnM7-06

### Surface Water Contact Angle of a HnM7-06 Protein Coated Glass Surface

Protein solutions (1 mg/mL HnM7-06, dissolved in 98% formic acid) were drop-cast on glass slides, followed by drying at room temperature overnight. The coated glass slides were fixed in a Theta Flow Tensiometer (Biolin Scientific, UK), The water contact angles were recorded continuously over 30 s.

The surface of untreated glass was hydrophilic, showing a water-contact angle of 22-24° that remained constant after 30 seconds of equilibration. Glass coated with HnM7-06 showed contact angles of 76-80°, which was maintained after 30 seconds equilibration. The inventors hereby demonstrate that HnM7-06 is a promising material for modifying the relative hydrophobicity of materials, such as may be desirable when used as a film or coating.

Co-Spinning HnM7-06 Protein with Nylon

Following the same methods as described in Example 8, HnM7-06-nylon fibers were wet-spun using a nylon dope solution in 98% formic acid (FA) at a nylon concentration of 15 wt. %. HnM7-06 was dissolved in FA followed by the addition of nylon, and continuous mixing and shaking were employed until clear solutions were achieved. Wet spun fibers made from these solutions were subjected to tensile strength testing.

### Tensile Strength of Co-Spun HnM7-06-Nylon Fibers

The mechanical properties of HnM7-06-nylon fibers were evaluated using a universal testing machine (UTM) (Agilent T150 USA) with a 0.5 N transducer. The fibers were mounted onto paper frames (10 mm windows), followed by stretching at a strain rate of 0.01 mm·s⁻¹. The nominal gauge length was fixed at 10 mm. Two samples were measured from each fiber.

TABLE 2
Tensile strength measurements of HnM7-06-nylon fibers

| | | Tensile | Elongation | |
|---|---|---|---|---|
| HnM7-06 | Modulus | strength | at break | Toughness |
| inclusion wt % | (MPa) | (MPa) | (%) | (MPa) |
| 0 | 125 | 18.69 | 197 | 22.19 |
| 1 | 673 | 23.61 | 289 | 54.09 |
| 2 | 930 | 40.43 | 275 | 89.74 |
| 5 | 1184 | 38.56 | 300 | 91.21 |
| 10 | 1021 | 35.32 | 314 | 87.46 |
| 20 | 971 | 42.16 | 158 | 56.59 |

As presented in Table 2, modulus, tensile strength, and toughness were increased when HnM7-06 protein was included in the nylon co-spun fibers. The elongation at break was also increased when HnM7-06 protein was included in the nylon co-spun fibers at levels up to and including 10 wt. %. The inventors hereby demonstrate that HnM7-06 protein improves the strength of nylon fibers imparting desirable properties to fibers that are used in numerous applications including textiles, woven and nonwoven materials.

### Example 8—HnP1 (SEQ ID NO: 4)

This example describes the production of an oligopeptide by chemical solid-phase synthesis in the production of peptide HnP1.

The peptide (SEQ ID NO: 4) was synthesized on pre-loaded 2-Cl-Trt resin, using standard Fmoc synthesis protocol with DIC/HOBt coupling, on an APEX 396 automatic synthesizer. The resin was swollen in DMF for 30 min, treated with 20v % Piperidine-DMF for 8 minutes to remove the Fmoc protecting group, at r.t., and washed with DMF for three times. For the coupling reaction, the resin was added with Fmoc-protected amino acid, HOBt, DIC and NMP. The mixture was vortexed for 20 minutes at r.t. Afterwards, the resin was washed with DMF once. The cycle of deprotection and coupling steps was repeated until the last amino acid residue was assembled. The resin was then washed with DMF, DCM and dried with air. The peptide was cleaved using a TFA cocktail (95v % TFA, 2.5v % water and 2.5v % TIS) for three hours. Crude peptide was precipitated by adding ice-chilled anhydrous ethyl ether, washed with anhydrous ethyl ether for three times, and dried in vacuo. After the synthesis, conventional prep-HPLC was used for peptide purification and salt conversion (HCl or acetate).

Co-Spinning HnP1 Peptide with Nylon

A nylon dope solution was prepared by dissolving nylon 6/6 pellets (molecular weight 252.35 daltons, Sigma-Aldrich, St Louis, MO, USA) in 98% formic acid (FA) and stirring overnight to give a clear solution (15% w/w nylon). Nylon-HnP1 combined dopes were made by first dissolving HnP1 in FA (at various concentrations) and then dissolving nylon in the HnP1 solutions. Solutions were filled inside a syringe, followed by a wet spinning process in a water coagulation bath to make single fibers for testing.

The morphology of the as-spun fibers was identified using scanning electron microscopy (SEM) The pristine nylon (0% HnP1) and HnP1-nylon fibers were coated by 5 nm of Pt conductive coating, followed by SEM imaging using Zeiss Supra 55VP field emission gun with an accelerating voltage of 5 KV, aperture size of 20 μm and a working distance from 7 to 8 mm.

Co-Spinning HnP1 with Silk

Bivoltine Bombyx mori reeled, un-degummed fibers (SRR Silk Reeling Unit; Ramanagara, Karnataka. India) were used for the preparation of regenerated silk. Lithium bromide (LiBr, 99%), sodium carbonate (Na2CO3, 99.5%), and ammonium sulphate ((NH₄)₂SO₄, 99%) (Sigma-Aldrich) were used for the dissolving, degumming, and coagulation of silk fibers, respectively. B. mori silk fibers were degummed in 500 ml aluminium pots in a Ahiba IR Pro rotary dyeing machine (Datacolor, Lawrenceville, USA) at a raw silk (g): liquor (ml) ratio of 1:50, for 30 minutes at 98° C. using 0.2% sodium carbonate. The degummed silk was washed several times with deionised water and then dried at 60° C. in a fan forced oven. For regenerated silk fibroin (RSF), sodium carbonate degummed silk was dissolved in 9.3M LiBr solution with a raw silk (g): liquor (ml) ratio of 1:7.5 at 60° C. for 40 minutes. The dissolved solution was then kept in a deionized water bath for dialysis, followed by concentration to get a concentrated aqueous silk solution. The silk solution was freeze dried for 48 hours to get pure solid regenerated silk crystals.

Similar methods as used when co-spinning with nylon were used to wet spin 12 wt % silk with different concentrations of HnP1. The as-spun fiber morphology was examined using SEM.

### SEM Result Discussion:

The measured fiber diameter of wet spun nylon, silk and HnP1-silk, HnP1-nylon fibers was found to be consistent around 60-70 μm. The fiber roughness gradually increased with increasing HnP1 amount from 1 to 10 wt. % with both nylon and silk. The increased surface roughness will increase the surface area to volume ratio. Without wishing to be bound by theory, the inventors believe these attributes are useful in applications such as functional textiles that benefit from enhanced reactive sites, adsorption, surface adhesion and heat transfer.

The cross-section morphology of nylon and HnP1-nylon, HnP1-silk fibers indicates the presence of highly porous structures as shown in Figure x and y. Interestingly, smaller more regular pores were generated with increasing the amount of HnP1 in the mixed matrix of HnP1-nylon and HnP1-silk from 1 to 10 wt. %, Smaller pores can lead to higher adsorption rates for other chemicals, high interfacial energy, and improved wetting properties. Without wishing to be bound by theory, the inventors believe these attributes will benefit applications that require cell-adhesion or water-based coating formulations for example, in the generation of functional textile materials.

### Surface Water Contact Angle Measurements for Fibers Containing HnP1.

Water contact angles (WCA) were measured for individual fibers made by co-spinning nylon or silk with HnP1 using a Theta Flow Tensiometer (Biolin Scientific, UK). WCA were recorded continuously over 30 s.

### Discussion of WCA Results

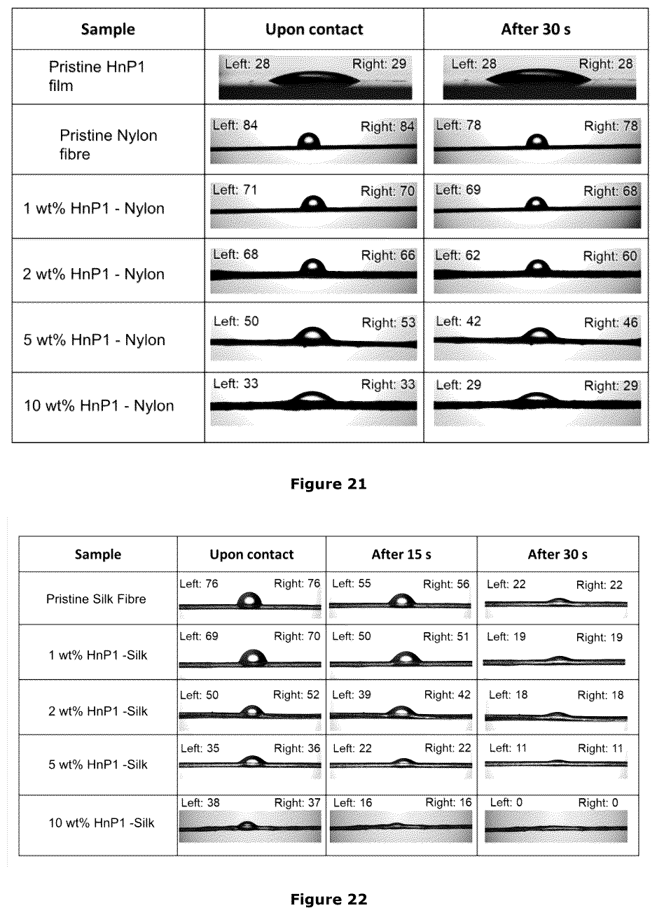

As presented in FIGS. 21 and 22, the WCA of nylon and of silk fibers was reduced when more HnP1 was included within the fiber composition, indicating an HnP1 concentration-dependent change in surface hydrophilicity (wettability).

These results show that incorporation of HnP1 acts to increase the hydrophilicity of both nylon and silk fibers. Without wishing to be bound by theory, the inventors believe that these attributes will provide useful benefits to applications that require wettable fibers such as in the creation of absorbent materials,

### Tensile Strength of HnP1-Nylon Co-Spun Fibers

The mechanical properties of HnP1-nylon fibers were evaluated using a universal testing machine (UTM) (Agilent T150 USA) with a 0.5 N transducer. The fibers were mounted onto paper frames (10 mm windows), followed by stretching at a strain rate of 0.01 mm·s⁻¹. The nominal gauge length was fixed at 10 mm. Two samples were measured from each fiber.

TABLE 3
Tensile strength measurements of HnP1-nylon fibers

| | | Tensile | Elongation | |
|---|---|---|---|---|
| HnP1 | Modulus | strength | at break | Toughness |
| inclusion % | (MPa) | (MPa) | (%) | (MPa) |
| 0 | 389 | 18.14 | 199 | 29.38 |
| 1 | 692 | 47.98 | 300 | 107.68 |
| 2 | 17848 | 76.52 | 317 | 214.72 |
| 5 | 3911 | 137.64 | 221 | 288.61 |

### Discussion of Tensile Strength Results

As presented in Table 3, the tensile strength of nylon was improved by the inclusion of HnP1 peptide in co-spun fibers.

The inventors hereby demonstrate that HnP1 peptide improves the strength of nylon fibers imparting useful and desirable properties to fibers for various applications such as use in textiles, woven and nonwoven materials.

TABLE 4
Nucleic acid and Amino acid sequences

| | | Nucleic | |
|---|---|---|---|
| | | Acid (NA) | |
| SEQ ID | | or Amino | |
| NO: | NUCLEOTIDE OR AMINO ACID PRIMARY SEQUENCE | Acid (AA) | Comments |
| 1 | ATGAAATGTGTCTTGTTATTAGTTGTCATATTCGCAGTC | NA | FUN |
| | GCGGCTTATGCCCGACCTCAGTCGCCACACCATGGTC | | polypeptide |
| | ACCATGGTGCGAAGACTACTTTGAAATTCCATCCCCAC | | CDNA |
| | CATGGATTCCGCAAACATCACGGCCATCCTAATCATCA | | |
| | TAAAGCTTCCAACAGCGAAAAAGAACTAGAATGGGATG | | |
| | ACAACGTACAGCAGAAATACTGGAATGAGAAACGACTC | | |
| | CAATCCGATAGAAAACATGAATCAGAATTCGAACAATC | | |
| | CAAATCCGAAAACAAAGTAACAAAATCGTCCAGTACAC | | |
| | ACTCTAGCCAACATAATGAGAACCATACGGAACTATCT | | |
| | GAGTCAAAATCCACACATACGGCACATAAATCGTCAGG | | |
| | AGGAAAATCAAGCCAAATGGAAAGCCAACACCAACAA | | |
| | GCTCAAAACCAACAGTCCCAAGCCCAGAGCCAAGAAC | | |
| | AGGAATCCCAGTCTGCAGAATCTGAATCCGCTGTTCAG | | |
| | TCAAGCCAATCCTCCAGTGGAATGAGCAGCCAAGCTCA | | |
| | AAGCCAACAACAACAAGCCCAGAGCCAAGAACAGGAA | | |
| | TCCCAGTCCTGCAGAATCAGAATCCGCTGTTCAGTCAA | | |
| | GCCAATCCTCCAGTGGAATGAGCAGCCAAGCTCAAAG | | |
| | CCAACAACAACAAGCCCAGAGCCAAGAACAGGAATCC | | |
| | CAGTCTGCAGAATCATGAGCAGCCAAGCTCAAAGCCA | | |
| | ACAACAGCAAGCTCAAGTCCAACAGTCTCAAGCTCAGA | | |
| | GCCAAGAACAGGAGTCCCAGTCTGCAGGATCTGAATC | | |
| | CGCCGTTCAGTCAAGCAAATCCTCCAGTGGAATGAGCA | | |
| | GCCAAGCTCAAAGCCAACAGTCCCAAGCTCAGAGCCA | | |
| | AGAACAGGAATCCCAATCTGCTCAATCTGGATCCGATG | | |
| | TTCAAGTAAGCAAATCCTCCAGTGGAAGGAGCAGCCA | | |
| | AGCTCAAAGCCAACAACAACAATCTCAGCTCCAACAGT | | |
| | CCCAAGCTCAGAGCCAAGAACAGGAATCCCAGTCTGC | | |
| | AGGATCTGAATCCGCCGTTCAGTCAAGCAAATCCTCCA | | |
| | GTGGAATGAGCAGCCAAGCTCAAAGCCAACAACAGCA | | |
| | AGCTCAAGTCCAACAGTCTCAAGCTCAGAGCCAAGAAC | | |
| | AGGAATCCCAATCTGCTCAATCTGAATCCGCCATTCAG | | |
| | TCAAGCAAATCCTCCAGTGGAATGAGCAGCCAAGCTCA | | |
| | AAGCCAACAACAGCAAGCTCAAGTCCAACAGTCTCAAG | | |
| | CTCAGAGCCAAGAACAGGAATCCCAATCTGCTCAATCT | | |
| | GAATCCGCCATTCAGTCAAGCAAATCCTCCAGTGGAAT | | |
| | GAGCAGCCAAGCTCAAAGCCAACAACAGCAAGCTCAA | | |
| | GTCCAACAGTCCCAAGCTCAAAGCCAAGAACAGGAAT | | |
| | CCCAGTCTGCAGAATCTGAATCCGATGTTCAGTCAAGC | | |
| | AAATCCTCCAGGGGAATGAGCAGCCATGCTCAAAGCC | | |
| | AACAGTCCCAAGCTCAGAGCCAAGAACAGGAATCCCA | | |
| | ATCTGCTCAATCTGGATCCGATGTTGAAGTAAGCAAAT | | |
| | CCTCCAGTGGAAGGAGCAGCCAAGCTCAAAGCCAACA | | |
| | ACAACAATCTCAGCTCCAACAGTCCCAAGCTCAGAGCC | | |
| | AAGAACAGGAATCCCAGTCTGCAGGATCTGAATCCGC | | |
| | CGTTCAGTCAAGCAAATCCTCCAGTGGAATGAGCAGCC | | |
| | AAGCTCAAAGCCAACAACAGCAAGCTCAAGTCCAACA | | |
| | GTCTCAAGCTCAGAGCCAAGAACAGGAATCCCAGTCT | | |
| | GCAGGATCTGAATCCGCCGTTCAGTCAAGCAAATCCTC | | |
| | CAGTGGAATGAGCAGCCAAGCTCAAAGCCAACAACAG | | |
| | CAAGCTCAAGTCCAACAGTCTCAAGCTCAGAGCCAAGA | | |
| | ACAGGAGTCACAGTCTGCAGGATCTGAATCCGCCGTT | | |
| | CAGTCAAGCAAATCCTCCAGTGGAATGAGCAGCCAAG | | |
| | CTCAAAGCCAACAACAACAAGCTCAGAGCCAAGAACA | | |
| | GGAATCCCAATCTGCTCAATCTGAATCCGCCATTCAGT | | |
| | CAAGCAAATCCTCCAGTGGAATGAGTAGCCAAGCTCAA | | |
| | AGCCAACAACAACAATCTCAGCTCCAACAGTCCCAAGC | | |
| | TCAGAGCCAAGAACAGGAATCCCAGTCTGCAGAATCT | | |
| | GAATCCGCCGTTCAGTCAAGCAAATCCTCCAGTGGAAT | | |
| | GAGCAGCCAAGCTCAAAGCCAACAACAACAAGCTCAG | | |
| | AGCCAAGAACAGGAATCGCAATCTGCTCAATCTGAATC | | |
| | CGCCATTCAGTCAAGCAAATCCTCCAGTGGAATGAGCA | | |
| | GCCAAGCTCAAAGCCAACAACAGCAAGCTCAAGTCCA | | |
| | ACAGTCCCAAGCTCAAAGCCAAGAACAGGAATCCCAG | | |
| | TCTGCAGAATCTGAATCCGATGTTCAGTCAAGCAAATC | | |
| | CTCCAGGGGAATGAGCAGCCATGCTCAAAGCCAACAG | | |
| | TCCCAAGCTCAGAGCCAAGAACAGGAATCCCAATCTGC | | |
| | TCAATCTGGATCCGATGTTGAAGTAAGCAAATCCTCCA | | |
| | GTGGAAGGAGCAGCCAAGCTCAAAGCCAACAACAACA | | |
| | ATCTCAGCTCCAACAGTCCCAAGCTCAGAGCCAAGAAC | | |
| | AGGAATCCCAGTCTGCAGGATCTGAATCCGCCGTTCA | | |
| | GTCAAGCAAATCCTCCAGTGGAATGAGCAGCCAAGCT | | |
| | CAAAGCCAACAACAGCAAGCTCAAGTCCAACAGTCTCA | | |
| | AGCTCAGAGCCAAGAACAGGAGTCCCAGTCTGCAGGA | | |
| | TCTGAATCCGCCGTTCAGTCAAGCAAATCCTCCAGTGG | | |
| | AATGAGCAGCCAAGCTCAAAGCCAACAACAACAAGCT | | |
| | CAGAGCCAAGAACAGGAATCCCAATCTGCTCAATCTGA | | |
| | ATCCGCCATTCAGTCAAGCAAATCCTCCAGTGGAATGA | | |
| | GCAGCCAAGCTCAAAGCCAACAACAACAATCTCAGCTC | | |
| | CAACAGTCCCAAGCTCAGAGCCAAGAACAGGAATCCC | | |
| | AGTCTGCAGAATCTGAATCCGCTGTTCAGTCAAGCAAA | | |
| | TCCTCCAGTGGAATGAGCAGCCAAGCTCAAAGCCAAC | | |
| | AACAACAAGCTCAGAGCCAAGAACAGGAATCCCAATCT | | |
| | GCTCAATCTGAATCCGCCATTCAGTCAAGCAAATCCTC | | |
| | CAGTGGAATGAGCAGCCAAGCTCAAAGCCAACAACAG | | |
| | CAAGCTCAAGTCCAACAGTCTCAAGCTCAGAGCCAAGA | | |
| | ACAGGAGTCCCAGTCTGCAGGATCTGAATCCGCCGTT | | |
| | CAGTCAAGCAAATCCTCCAGTGGAATGAGCAGCCAAG | | |
| | CTCAAAGCCAACAACAACAAGCTCAGAGCCAAGAACA | | |
| | GGAATCCCAATCTGCTCAATCTGAATCCGCCATTCAGT | | |
| | CAAGCAAATCCTCCAGTGGAATGAGTAGCCAAGCTCAA | | |
| | AGCCAACAACAACAATCTCAGCTCCAACAGTCCCAAGC | | |
| | TCAGAGCCAAGAACAGGAATCCCAGTCTGCAGAATCT | | |
| | GAATCCGCTGTTCAGTCAAGCAAATCCTCCAGTGGAAT | | |
| | GAGCAGCCAAGCTCAAAGCCAACAACAACAAGCTCAG | | |
| | AGCCAAGAACAGGAATCGCAATCTGCTCAATCTGAATC | | |
| | CGCCATTCAGTCAAGCAAATCCTCCAGTGGAATGAGCA | | |
| | GCCAAGCTCAAAGCCAACAACAGCAAGCTCAAGTCCA | | |
| | ACAGTCCCAAGCTCAAAGCCAAGAACAGGAATCCCAG | | |
| | TCTGCAGAATCTGAATCCGATGTTCAGTCAAGCAAATC | | |
| | CTCCAGGGGAATGAGCAGCCATGCTCAAAGCCACCAG | | |
| | TCCCAAGCTCAGAGCCAAGAACAGGAATCCCAATCTGC | | |
| | TCAATCTGGATCCGATGTTGAAGTAAGCAAATCCTCCA | | |
| | GTGGAAGGAGCAGCCAAGCTCAAAGCCAACAACAACA | | |
| | ATCTCAGCTCCAACAGTCCCAAGCTCAGAGCCAAGAAC | | |
| | AGGAATCCCAGTCTGCAGGATCTGAATCCGCCGTTCA | | |
| | GTCAAGCAAATCCTCCAGTGGAATGAGCAGCCAAGCT | | |
| | CAAAGCCAACAACAACAAGCTCAAGTCCAACAGTCTCA | | |
| | AGCTCAGAGCCAAGAACAGGAGTCTCAAGCAAATCCT | | |
| | CCAGTGGAAATGAGCAGCCAAGCTCAAAGCCAACAAC | | |
| | AGCAATCTCAACTGCAACAGTCCCAAGCTCAGAGCCAA | | |
| | GAACAGGAATCTCAATCCGACCAGGCTGAATCCGATGT | | |
| | TCAGTCAAGCAAATCCTCCAGGGGAATGAGCAGCCAC | | |
| | GCTCAAAGCCAACAGTCTCAAGCTCAAAGCCAGGAAC | | |
| | AGGAATCCCAGTCAGCAGAATCTGAGTCCGCTGTTCA | | |
| | GTCAAGCAAATCCTCCAGTGGAAGGAGCAGCCAAGCT | | |
| | CAAAGCCAACAACAACAATCTCAGCTCCAACAGTCCCA | | |
| | AGCTCAGAGCCAAGAACAGGAATCCCAATCTGCTCAAT | | |
| | CTGCATCCGATGTTGAAGTAAGCAAATCCTCCAGTGGA | | |
| | AGGAGCAGCCAAGCTCAAAGCCAACAACAACAATCTC | | |
| | AGCTCCAACAGTCCCAAGCTCAAAGCCAAGAACAGGA | | |
| | ATCCCAGTCTGCAGAATCTGAATCCGATGTTCAGTCAA | | |
| | GCAAATCCTCCAGGGGAATGAGCAGCCATGCTCAAAG | | |
| | CCAACAGTCCCAAGCTCAGAGCCAAGAACAGGAATCC | | |
| | CAATCTGCTCAATCTGGATCCGATGTTGAAGTAAGCAA | | |
| | ATCCTCCAGTGGAAGGAGCAGCCAAGCTCAAAGCCAA | | |
| | CAACAACAATCTCAGCTCCAACAGTCCCAAGCTCAGAG | | |
| | CCAAGAACAGGAATCCCAGTCTGCAGGATCTGAATCC | | |
| | GCCGTTCAGTCAAGCAAATCCTCTCGTGGAATGAGCA | | |
| | GCCAAGCTCAAAGCCAACAACAACAATCTCAGCTCCAA | | |
| | CAGTCTCAAGCTCAGAGCCAAGAACAGGAATCCCAATC | | |
| | TGCTCAATCTGGATCCGATGTTGAAGTAAGCAAATCCT | | |
| | CCAGTGGAAGGAGCAGCCAAGCTCAAAGCCAACAACA | | |
| | ACAATCTCAGCTCCAACAGTCCCAAGCTCAAAGCCAAG | | |
| | AACAGGAATCCCAGTCTGCAGAATCTGAATCCGATGTT | | |
| | CAGTCTAGCAAATCCTCCAGGGGAATGAGCAGCCATG | | |
| | CTCAAAGCCAACAGTCTCAAGCTCAAAGCCAGGAACA | | |
| | GGAATCCCAGTCAGCAGAATCTGAATCCGCCGTTCAGT | | |
| | CAAGCAAATCCTCTCGTGGAATGAGCAGCCAAGCTCAA | | |
| | AGCCAACAACAACAATCTCAGCTCCAACAGTCTCAAGC | | |
| | TCAGAGCCAAGAACAGGAATCCCAATCTGCTCAATCTG | | |
| | GATCCGATGTTGAAGTAAGCAAATCCTCCAGTGGAAG | | |
| | GAGCAGCCAAGCTCAAAGCCAACAACAACAATCTCAG | | |
| | CTCCAACAGTCCCAAGCTCAGAGCCAAGAACAGGAAT | | |
| | CCCAGTCTGCAGGATCTGAATCCGCCGTTCAGTCAAGC | | |
| | AAATCCTCCAGTGGAATGAGCAGCCAAGCTCAAAGCC | | |
| | AACAACAGCAATCTCAACTCCAACAGTCCCAAGCTCAG | | |
| | AGCCAAGAACAGGAATCTCAATCCGACCAGGCTGAAT | | |
| | CCGATGTTCAGTCAAGCAAATCCTCCAGGGGAATGAG | | |
| | CAGCCACGCTCAAAGCCAACAGTCTCAAGCTCAAAGCC | | |
| | AGGAACAGGAATCCCAGTCAGCAGAATCTGAATCCGC | | |
| | TGTTCAGTCAAGCAAATCCTCCAGTGGAAGGAGCAGC | | |
| | CAAGCTCAAAGCCAACAACAACAATCTCAGCTCCAACA | | |
| | GTCTCAAGCTCAGAGCCAAGAACAGGAATCCCAATCTG | | |
| | CTCAATCTGGATCCGATGTTGAAGTAAGCAAATCCTCC | | |
| | AGTGGAAGGAGCAGCCAAGCTCAAAGCCAACAACAAC | | |
| | AATCTCAGCTCCAACAGTCCCAAGCTCAGAGCCAAGAA | | |
| | CAGGAATCCCAGTCTGCAGGATCTGAATCCGCCGTTCA | | |
| | GTCAAGCAAATCCTCTCAGTGGAATGAGCAGCCAAGCT | | |
| | CAAAGCCAACAACAACAATCTCAGCTCCAACAGTCTCA | | |
| | AGCTCAGAGCCAAGAACAGGAATCCCAATCTGCTCAAT | | |
| | CTGGATCCGATGTATGTTGAAGTCAAGCAAATCCTCCA | | |
| | GTGGAAGGAGCAGCCAAGCTCAAAGCCAACAACAACA | | |
| | ATCTCAGCTCCAACAGTCCCAAGCTCAAAGCCAAGAAC | | |
| | AGGAATCCCAGTCTGCAGAATCTGAATCCGATGTTCAG | | |
| | TCAAGCAAATCCTCCAGGGGAATGAGCAGCCATGCTC | | |
| | AAAGCCAACAGTCTCAAGCTCAAAGCCAGGAACAGGA | | |
| | ATCCCAGTCAGCAGAATCTGAATCCGCCGTTCAGTCAA | | |
| | GCAAATCCTCTCGTGGAATGAGCAGCCAAGCTCAAAG | | |
| | CCAACAACAACAATCTCAGCTCCAACAGTCTCAAGCTC | | |
| | AGAGCCAAGAACAGGAATCCCAATCTGCTCAATCTGGA | | |
| | TCCGATGTTGAAGTAAGCAAATCCTCCAGTGGAAGGA | | |
| | GCAGCCAAGCTCAAAGCCAACAACAACAATCTCAGCTC | | |
| | CAACAGTCCCAAGCTCAAAGCCAAGAACAGGAATCCC | | |
| | AGTCTGCAGAATCTGAATCCGATGTTCAGTCAAGCAAA | | |
| | TCCTCCAGGGGAATGAGCAGCCGTGCTCAAAGCCAAC | | |
| | AGTCCCAAGCTCAGAGCCAAGAACAGGAATCCCAATCT | | |
| | GCTCAATCTGGATCCGATGTTGAAGTAAGCAAATCCTC | | |
| | CAGTGGAAGGAGCAGCCAAGCTCAAAGCCAACAACAA | | |
| | CAATCTCAGCTCCAACAGTCCCAAGCTCAGAGCCAAGA | | |
| | ACAGGAATCCCAGTCTGCAGGATCTGAATCCGCCGTTC | | |
| | AGTCAAGCAAATCCTCTCAGTGGAATGAGCAGCCAAG | | |
| | CTCAAAGCCAACAACAACAATCTCAGCTCCAACAGTCC | | |
| | CAAGCTCAGAGCCAAGAACAGGAATCCCAGTCTGCAG | | |
| | GATCATGAGCAGCCAAGCTCAAAGCCAACAACAACAA | | |
| | GCTCAACTCCAACAGTCCCAAGCTCAAAGCCAAGAACA | | |
| | GGAATCCCAGTCTGCAGAATCTGAATCCGATGTTCAGT | | |
| | CAAGCAAATCCTCCAGGGGAATGAGCAGCCGTGCTCA | | |
| | AAGCCAACAGTCCCAAGCTCAGAGCCAAGAACAGGAA | | |
| | TCCCAATCTGCTCAATCTGGATCCGATGTTGAAGTAAG | | |
| | CAAATCCTCCAGTGGAAGGAGCAGCCAAGCTCAAAGC | | |
| | CAACAACAACAATCTCAGCTCCAACAGTCCCAAGCTCA | | |
| | AAGCCAAGAACAGGAATCCCAGTCTGCAGAATCTGAAT | | |
| | CCGATGTTCAGTCAAGCAAATCCTCCAGGGGAATGAG | | |
| | CAGCCATGCTCAAAGCCAACAGTCTCAAGCTCAAAGCC | | |
| | AGGAACAGGAATCCCAGTCAGCAGAATCTGAATCCGC | | |
| | CGTTCAGTCAAGCAAATCCTCTCGTGGAATGAGCAGCC | | |
| | AAGCTCAAAGCCAACAACAACAATCTCAGCTCCAACAG | | |
| | TCTCAAGCTCAGAGCCAAGAACAGGAATCCCAATCTGC | | |
| | TCAATCTGGATCCGATGTTGAAGTAAGCAAATCCTCCA | | |
| | GTGGAAGGAGCAGCCAAGCTCAAAGCCAACAACAACA | | |
| | ATCTCAGCTCCAACAGTCCCAAGCTCAAAGCCAAGAAC | | |
| | AGGAATCCCAGTCTGCAGAATCTGAATCCGATGTTCAG | | |
| | TCAAGCAAATCCTCCAGGGGAATGAGCAGCCATGCTC | | |
| | AAAGCCAACAGTCCCAAGCTCAGAGCCAAGAACAGGA | | |
| | ATCCCAATCTGCTCAATCTGGATCCGATGTTGAAGTAA | | |
| | GCAAATCCTCCAGTGGAAGGAGCAGCCAAGCTCAAAG | | |
| | CCAACAACAACAATCTCAGCTCCAACAGTCCCAAGCTC | | |
| | AAAGCCAAGAACAGGAATCCCAGTCTGCAGAATCTGA | | |
| | ATCCGATGTTCAGTCAAGCAAATCCTCCAGGGGAATGA | | |
| | GCAGCCGTGCTCAAAGCCAACAGTCCCAAGCTCAGAG | | |
| | CCAAGAACAGGAATCCCAATCTGCTCAATCTGGATCCG | | |
| | ATGTTGACAGTAAGCAAATCCTCCAGTGGAAGGAGCA | | |
| | GCCAAGCTCAAAGCCAACAACAACAATCTCAGCTCCAA | | |
| | CAGTCCCAAGCTCAGAGCCAAGAACAGGAATCCCAGT | | |
| | CTGCAGGATCATGAGCAGCCAAGCTCAAAGCCAACAA | | |
| | CAACAATCTCAGCTCCAACAGTCCCAAGCTCAGAGCCA | | |
| | AGAACAGGAATCCCAATCTGCTCAATCTGGATCCGATG | | |
| | TTGAAGTAAGCAAATCCTCCAGTGGAAGGAGCAGCCA | | |
| | AGCTCAAAGCCAACAACAACAATCTCAGCTCCAACAGT | | |
| | CCCAAGCTCAGAGCCAAGAACAGGAATCCCAGTCTGC | | |
| | AGAATCTGAATCCGCTGTTCAGTCAAGCAAATCCTCCA | | |
| | GTGGAATGAGCAGCCAAGCTCAAAGCCAACAACAACA | | |
| | ATCTCAACTCCAACAGTCCCAAGCTCAAAGCCAGGAAC | | |
| | AGGAATCTCAATCCGACCAGGCTGAATCAGATGTTCAG | | |
| | TCAAGCAAATCCTCCAGGGGAATGAGCAGCCATGCTC | | |
| | AAAGCCAACAGTCTCAAGCTCAAAGCCAGGAACAGGA | | |
| | ATCCCAGTCAGCAGAATCTGAATCCGCCGTTCAGTCAA | | |
| | GCAAATCCTCTCGTGGAATGAGCAGCCAAGCTCAAAG | | |
| | CCAACAACAACAATCTCAGCTCCAACAGTCTCAAGCTC | | |
| | AGAGCCAAGAACAGGAATCCCAATCTGCTCAATCTGGA | | |
| | TCCGATGTTGAAGTAAGCAAATCCTCCAGTGGAAGGA | | |
| | GCAGCCAAGCTCAAAGCCAACAACAACAATCTCAGCTC | | |
| | CAACAGTCCCAAGCTCAAAGCCAAGAACAGGAATCCC | | |
| | AGTCTGCAGAATCTGAATCCGATGTTCAGTCAAGCAAA | | |
| | TCCTCCAGGGGAATGAGCAGCCGTGCTCAAAGCCAAC | | |
| | AGTCCCAAGCTCAGAGCCAAGAACAGGAATCCCAATCT | | |
| | GCTCAATCTGGATCCGATGTTGACGTAAGCAAATCCTC | | |
| | CAGTGGAAGGAGCAGCCAAGCTCAAAGCCAACAACAA | | |
| | CAATCTCAGCTCCAACAGTCCCAAGCTCAGAGCCAAGA | | |
| | ACAGGAATCCCAGTCTGCAGGATCTGAATCCGCCGTTC | | |
| | AGTCAAGCAAATCCTCTCGTGGAATGAGCAGCCAAGCT | | |
| | CAAAGCCAACAACAACAATCTCAGCTCCAACAGTCCCA | | |
| | AGCTCAGAGCCAAGAACAGGAATCCCAGTCTGCAGGA | | |
| | TCTGAATCCGCTGTTCAGTCAAGCAAATCCTCTCAGTG | | |
| | GAATGAGCAGCCAAGCTCAAAGCCAACAACAACAAGC | | |
| | TCAACTCCAACAGTCCCAAGCTCAAAGCCAAGAACAGG | | |
| | AATCCCAGTCTGCAGAATCATGAGCAGCCGTGCTCAAA | | |
| | GCCAACAGTCCCAAGCTCAGAGCCAAGAACAGGAATC | | |
| | CCAATCTGCTCAATCTGGATCCGATGTTGAAGTAAGCA | | |
| | AATCCTCCAGTGGAAGGAGCAGCCAAGCTCAAAGCCA | | |
| | ACAACAACAATCTCAGCTCCAACAGTCCCAAGCTCAAA | | |
| | GCCAAGAACAGGAATCCCAGTCTGCAGAATCTGAATCC | | |
| | GATGTTCAGTCAAGCAAATCCTCCAGGGGAATGAGCA | | |
| | GCCATGCTCAAAGCCAACAGTCTCAAGCTCAAAGCCAG | | |
| | GAACAGGAATCCCAGTCAGCAGAATCTGAATCCGCCG | | |
| | TTCAGTCAAGCAAATCCTCTCGTGGAATGAGCAGCCAA | | |
| | GCTCAAAGCCAACAACAACAATCTCAGCTCCAACAGTC | | |
| | TCAAGCTCAGAGCCAAGAACAGGAATCCCAATCTGCTC | | |
| | AATCTGGATCCGATGTTGAAGTAAGCAAATCCTCCAGT | | |
| | GGAAGGAGCAGCCAAGCTCAAAGCCAACAACAACAAT | | |
| | CTCAGCTCCAACAGTCCCAAGCTCAAAGCCAAGAACAG | | |
| | GAATCCCAGTCTGCAGAATCTGAATCCGATGTTCAGTC | | |
| | AAGCAAATCCTCCAGGGGAATGAGCAGCCATGCTCAA | | |
| | AGCCAACAGTCCCAAGCTCAGAGCCAAGAACAGGAAT | | |
| | CCCAATCTGCTCAATCTGGATCCGATGTTGAAGTAAGC | | |
| | AAATCCTCCAGTGGAAGGAGCAGCCAAGCTCAAAGCC | | |
| | AACAACAACAATCTCAGCTCCAACAGTCCCAAGCTCAG | | |
| | AGCCAAGAACAGGAATCCCAGTCTGCAGGATCTGAAT | | |
| | CCGCCGTTCAGTCAAGCAAATCCTCTCGTGGAATGAGC | | |
| | AGCCAAGCTCAAAGCCAACAACAACAATCTCAGCTCCA | | |
| | ACAGTCCCAAGCTCAGAGCCAAGAACAGGAATCCCAG | | |
| | TCTGCAGGATCTGAATCCGCCGTTCAGTCAAGCAAATC | | |
| | CTCTCAGTGGAATGAGCAGCCAAGCTCAAAGCCAACA | | |
| | ACAACAAGCTCAACTCCAACAGTCCCAAGCTCAGAGCC | | |
| | AAGAACAGGAATCCCAATCTGCTCAATCTGGATCCGAT | | |
| | GTATGAGCAGCCATGCTCAAAGCCAACAGTCTCAAGCT | | |
| | CAAAGCCAGGAACAGGAATCCCAGTCAGCAGAATCTG | | |
| | AATCCGCCGTTCAGTCAAGCAAATCCTCTCGTGGAATG | | |
| | AGCAGCCAAGCTCAAAGCCAACAACAACAATCTCAGCT | | |
| | CCAACAGTCTCAAGCTCAGAGCCAAGAACAGGAATCC | | |
| | CAGTCTGCAGAATCTGGATCCGCTGTTGCAGTCAAGCA | | |
| | AATCCTCCAGTGGAAGGAGCAGCCAAGCTCAAAGCCA | | |
| | ACAACAACAATCTCAGCTCCAACAGTCCCAAGCTCAGA | | |
| | GCCAAGAACAGGAATCCCAATCTGCTCAATCTGGATCC | | |
| | GATGTATGAGCAGCCATGCTCAAAGCCAACAGTCTCAA | | |
| | GCTCAAAGCCAGGAACAGGAATCCCAGTCTGCAGAAT | | |
| | CTGAATCCGATGTTCAGTCAAGCAAATCCTCCAGGGGA | | |
| | ATGAGCAGCCATGCTCAAAGCCAACAGTCCCAAGCTCA | | |
| | GAGCCAAGAACAGGAATCCCAATCTGCTCAATCTGGAT | | |
| | CCGATGTTGAAGTAAGCAAATCCTCCAGTGGAAGGAG | | |
| | CAGCCAAGCTCAAAGCCAACAACAACAATCTCAGCTCC | | |
| | AACAGTCCCAAGCTCAGAGCCAAGAACAGGAATCCCA | | |
| | GTCTGCAGGATCTGAATCCGCCGTTCAGTCAAGCAAAT | | |
| | CCTCTCGTGGCATGAGCAGCCAAGCTCAAAGCCAACA | | |
| | ACAACAATCTCAACTCCAACAGTCCCAAGCTCAAAGCC | | |
| | AGGAACAGGAATCTCAATCCGACCAGGCTGAATCAGA | | |
| | TGTTCAGTCAAGCAAATCCTCCAGGGGAATGAGCAGC | | |
| | CATGCTCAAAGCCAACAGTCTCAAGCTCAAAGCCAGGA | | |
| | ACAGGAATCCCAGTCAGCAGAATCTGAATCCGCCGTTC | | |
| | AGTCAAGCAAATCCTCTCGTGGAATGAGCAGCCAAGCT | | |
| | CAAAGCCAACAACAACAATCTCAGCTCCAACAGTCTCA | | |
| | AGCTCAGAGCCAAGAACAGGAATCCCAATCTGCTCAAT | | |
| | CTGGATCCGATGTTGAAGTAAGCAAATCCTCCAGTGGA | | |
| | AGGAGCAGCCAAGCTCAAAGCCAACAACAACAATCTC | | |
| | AGCTCCAACAGTCCCAAGCTCAGAGCCAAGAACAGGA | | |
| | ATCCCAATCTGCTCAATCTGAATCCGATGTTCAGTCAA | | |
| | GCAAATCCTCTCAGTGGAATGAGCAGCCAAGCTCAAA | | |
| | GCCAACAACAACAAGCTCAACTCCAACAGTCCCAAGCT | | |
| | CAAAGCCAAGAACAGGAATCCCAGTCTGCAGAATCAT | | |
| | GAGCAGCCAAGCTCAAAGCCAGCAACAACAATCTCAA | | |
| | CTCCAACAGTCCCAAGCTCAAAGCCAGGAACAGGAAT | | |
| | CCCAGTCAGCAGAATCTGAGTCCGCTGTTCAGTCAAGC | | |
| | AAATCCTCCAGTGGAATGAGCAGCCAAGCTCAAAGCC | | |
| | AACAACAGCAATCCCAACTGCAACAGTCCCAAGCTCAG | | |
| | AGCCAAGAACAGGAATCTCAATCCGACCAGGCTGAAT | | |
| | CAGATGTTCAGTCAAGCAAATCCTCCAGGGGAATGAG | | |
| | CAGCCAAGCTCAAAGCCAACAGTCTCAAGCTCAAAGCC | | |
| | AGGAACAGGAATCCCAGTCAGCAGAATCTGAATCCGC | | |
| | CGTTCAGTCAAGCAAATCCTCCAGTGGAATGAGCAGCC | | |
| | ATGCTCAAAGCCAACAGTCTCAAGCTCAAAGCCAGGAA | | |
| | CAGGAATCCCAGTCAGCAGAATCTGAATCCGCCGTTCA | | |
| | GTCAAGCAAATCCTCTCGTGGAATGAGCAGCCAAGCTC | | |
| | AAAGCCAACAACAACAATCTCAGCTCCAACAGTCTCAA | | |
| | GCTCAGAGCCAAGAACAGGAATCCCAATCTGCTCAATC | | |
| | TGGATCCGATGTTGAAGTAAGCAAATCCTCCAGTGGAA | | |
| | GGAGCAGCCAAGCTCAAAGCCAACAACAACAATCTCA | | |
| | GCTCCAACAGTCCCAAGCTCAGAGCCAAGAACAGGAA | | |
| | TCCCAGTCTGCAGGATCTGAATCCGCCGTTCAGTCAAG | | |
| | CAAATCCTCCAGTGGAATGAGCAGCCAAGCTCAAAGC | | |
| | CAACAACAACAATCTCAACTCCAACAGTCCCAAGCTCA | | |
| | AAGCCAAGAACAGGAATCCCAGTCTGCAGAATCTGAAT | | |
| | CCGATGTTCAGTCAAGCAAATCCTCCAGGGGAATGAG | | |
| | CAGCCATGCTCAAAGCCAACAGTCCCAAGCTCAGAGC | | |
| | CAAGAACAGGAATCCCAATCTGCTCAATCTGGATCCGA | | |
| | TGTTGAAGTAAGCAAATCCTCCAGTGGAAGGAGCAGC | | |
| | CAAGCTCAAAGCCAACAACAACAATCTCAGCTCCAACA | | |
| | GTCCCAAGCTCAGAGCCAAGAACAGGAATCCCAGTCT | | |
| | GCAGGATCTGAATCCGCCGTTCAGTCAAGCAAATCCTC | | |
| | TCAGTGGAATGAGCAGCCAAGCTCAAAGCCAACAACA | | |
| | ACAAGCTCAACTCCAACAGTCCCAAGCTCAGAGCCAAG | | |
| | AACAGGAATCCCAGTCTGCAGAATCATGAGCAGCCAT | | |
| | GCTCAAAGCCAACAGTCTCAAGCTCAAAGCCAGGAAC | | |
| | AGGAATCCCAGTCAGCAGAATCTGAATCCGCTGTTCAG | | |
| | TCAAGCAAATCCTCCAGTGGAATGAGCAGCCAAGCTCA | | |
| | AAGCCAACAACAACAATCTCAGCTCCAACAGTCTCAAG | | |
| | CTCAGAGCCAAGAACAGGAATCCCAATCTGCTCAATCT | | |
| | GGATCCGATGTTGAAGTAAGCAAATCCTCCAGTGGAA | | |
| | GGAGCAGCCAAGCTCAAAGCCAACAACAACAATCTCA | | |
| | GCTCCAACAGTCCCAAGCTCAGAGCCAAGAACAGGAA | | |
| | TCCCAGTCTGCAGGATCTGAATCCGCCGTTCAGTCAAG | | |
| | CAAATCCTCCAGTGGAATGAGCAGCCAAGCTCAAAGC | | |
| | CAACAACAACAATCTCAGCTCCAACAGTCCCAAGCTCA | | |
| | GAGCCAAGAACAGGAATCCCAGTCTGCAGGATCTGAA | | |
| | TCCGCCGTTGAGTCAAGCAAATCCTCTCGTGGAATGAG | | |
| | CAGCCATGCTCAAAGCCAACAATCTCAAGCTCAAAGCC | | |
| | AGGAACAGGAATCCCAGTCTGCAGGATCTGAATCCGC | | |
| | CGTTCAGTCAAGCAAATCCTCCAGTGGAATGAGCAGCC | | |
| | AAGCTCAAAGCCAACAACAACAAGCTCAACTCCAACAG | | |
| | TCCCAAGCTCAGAGCCAAGAACAGGAATCCCAGTCTG | | |
| | CAGGATCTGAATCCGCCGTTCAGTCAAGCAAATCCTCC | | |
| | AGTGGAATGAGCAGCCAAGCTCAAAGCCAACAACAAC | | |
| | AAGCTCAACTCCAACAGTCCCAAGCTCAGAGCCAAGAA | | |
| | CAGGAATCCCAGTCTGCAGAATCTGAATCCGCCGTTCA | | |
| | GTCAAGCAAATCCTCCAGTGGAATGAGCAGCCAAGCT | | |
| | CAAAGCCAACAACAACAATCTCAGCTCCAACAGTCCCA | | |
| | AGCTCAGAGCCAAGAACAGGAATCCCAGTCTGCAGAA | | |
| | TCTGAATCCGCTGTTCAGTCAAGCAAATCCTCCAGTGG | | |
| | AATGAGCAGCCAAGCTCAAAGCCAACAACAACAAGCT | | |
| | CAACTCCAACAGTCCCAAGCTCAGAGCCAAGAACAGG | | |
| | AATCCCAGTCTGCAGGATCTGAATCCGCCGTTCAGTCA | | |
| | AGCAAATCCTCCAGTGGAATGAGCAGCCAAGCTCAAA | | |
| | GCCAACAACAACAAGCTCAACTCCAACAGTCCCAAGCT | | |
| | CAGAGCCAAGAACAGGAATCCCAGTCTGCAGGATCTG | | |
| | AATCCGCCGTTCAGTCAAGCAAATCCTCCAGTGGAATG | | |
| | AGCAGCCAAGCTCAAAGCCAACAACAACAATCTCAGCT | | |
| | CCAACAGTCCCAAGCTCAGAGCCAAGAACAGGAATCG | | |
| | CAGTCTGCAGAATCTGAATCCGCCGTTGAGTCAAGCAA | | |
| | ATCCTCTGGAATGAGCAGCCATGCTCAAAGCCAACAAT | | |
| | CTCAAGCTCAAAGCCAGGAACAGGAATCCCAGTCTGC | | |
| | AGGATCTGAATCCGCCGTTCAGTCAAGCAAATCCTCCA | | |
| | GTGGAATGAGCAGCCAAGCTCAAAGCCAACAACAACA | | |
| | AGCTCAACTCCAACAGTCCCAAGCTCAGAGCCAAGAAC | | |
| | AGGAATCCCAGTCTGCAGGATCTGAATCCGCCGTTCA | | |
| | GTCAAGCAAATCCTCCAGTGGAATGAGCAGCCAAGCT | | |
| | CAAAGCCAACAACAACAAGCTCAACTCCAACAGTCCCA | | |
| | AGCTCAGAGCCAAGAACAGGAATCCCAGTCTGCAGAA | | |
| | TCTGAATCCGCCGTTCAGTCAAGCAAATCCTCCAGTGG | | |
| | AATGAGCAGCCAAGCTCAAAGCCAACAACAACAATCTC | | |
| | AGCTCCAACAGTCCCAAGCTCAGAGCCAAGAACAGGA | | |
| | ATCCCAGTCTGCAGGATCTGAATCCGCCGTTCAGTCAA | | |
| | GCAAATCCTCCAGTGGAATGAGCAGCCAAGCTCAAAG | | |
| | CCAACAACAACAAGCTCAACTCCAACAGTCCCAAGCTC | | |
| | AGAGCCAAGAACAGGAATCCCAGTCTGCAGGATCTGA | | |
| | ATCAGCCGTTCAGTCAAGCAAATCCTCCAGTGGAATGA | | |
| | GCAGCCAAGCTCAAAGCCAACAACAACAATCTCAGCTC | | |
| | CAACAGTCCCAAGCTCAGAGCCAAGAACAGGAATCGC | | |
| | AGTCTGCAGAATCTGAATCCGCCGTTGAGTCAAGCAAA | | |
| | TCCTCTCGTGGAATGAGCAGCCATGCTCAAAGCCAACA | | |
| | ATCTCAAGCTCAAAGCCAGGAACAGGAATCCCAGTCTG | | |
| | CAGGATCTGAATCCGCCGTTCAGTCAAGCAAATCCTCC | | |
| | AGTGGAATGAGCAGCCAAGCTCAAAGCCAACAACAAC | | |
| | AAGCTCAACTCCAACAGTCCCAAGCTCAGAGCCAAGAA | | |
| | CAGGAATCCCAGTCTGCAGGATCTGAATCAGCCGTTCA | | |
| | GTCAAGCAAATCCTCCAGTGGAATGAGCAGTCAAGCTC | | |
| | AAAGCCAACAGTCTCAAGCTCAAAGCCAGGAACAGGA | | |
| | ATCCCAATCTGCTCAATCTGGATCCGACGTTCAAGTAA | | |
| | GCAAATCGTCCAGTGGAATGAGCAGCCAAGCTCAAAG | | |
| | CCAACAACAGCAAGCTCAAGTCCAACAGTCTCAAGCTC | | |
| | AGAGCCAAGAACAGGAATCCCAATCTGCTCAATCTGAA | | |
| | TCCGCCATTCAGTCAAGCAAATCCTCCAGTGGAATGAG | | |
| | CAGCCAAGCTCAAAGCCAACAACAGCAAGCTCAAGTC | | |
| | CAACAGTCTCAAGCTCAGAGCCAAGAACAGGAATCCC | | |
| | AATCTGCTCAATCTGGATCCGACGTTCAAGTAAGCAAA | | |
| | TCCTCCAGTGGTAGGAGCAGCCAAGCTCAAAGCCAAC | | |
| | AACAACAATCTCAAGTCCAACAGTCCCAAGCTCAGAGC | | |
| | CAAGAACAGGAATCCCAGTCTGCAGGATCTGAATCAG | | |
| | CCGTTCAGTCAAGCAAATCCTCCAGTGGAATGAGCAGC | | |
| | CAAGCTCAAAGCCAACAACAACAATCTCAGCTCCAACA | | |
| | GTCCCAAGCTCAGAGCCAAGAACAGGAATCGCAGTCT | | |
| | GCAGAATCTGAATCCGCCGTTGAGTCAAGCAAATCCTC | | |
| | TCGTGGAATGAGCAGCCATGCTCAAAGCCAACAGTCTC | | |
| | AAGCTCAAAGCCAGGAACAGGAATCCCAGTCTGCAGG | | |
| | ATCTGAATCCGCCGTTCAGTCAAGCAAATCCTCCAGTG | | |
| | GAATGAGCAGCCAAGCTCAAAGCCAACAACAACAAGC | | |
| | TCAACTCCAACAGTCCCAAGCTCAGAGCCAAGAACAG | | |
| | GAATCCCAGTCTGCAGGATCTGAATCCGCCGTTCAGTC | | |
| | AAGCAAATCCTCCAGTGGAATGAGCAGCCAAGCTCAA | | |
| | AGCCAACAACAACAAGCTCAACTCCAACAGTCCCAAGC | | |
| | TCAGAGCCAAGAACAGGAATCCCAGTCTGCAGGATCT | | |
| | GAATCAGCCGTTCAGTCAAGCAAATCCTCCAGTGGAAT | | |
| | GAGCAGTCAAGCTCAAAGCCAACAGTCTCAAGCTCAAA | | |
| | GCCAGGAACAGGAATCCCAATCTGCTCAATCTGGATCC | | |
| | GACGTTCAAGTCAAGCAAATCCTCCAGTGGAATGAGCA | | |
| | GCCAAGCTCAAAGCCAACAACAGCAAGCTCAAGTCCA | | |
| | ACAGTCTCAAGCTCAGAGCCAAGAACAGGAATCCCAAT | | |
| | CTGCTCAATC | | |
| 2 | MKCVLLLVVIFAVAAYARPQSPHHGHHGAKTTLKFHPHH | AA | FUN |
| | GFRKHHGHPNHHKASNSEKELEWDDNVQQKYWNEKRL | | polypeptide |
| | QSDRKHESEFEQSKSENKVTKSSSTHSSQHNENHTELS | | predicted AA |
| | ESKSTHTAHKSSGGKSSQMESQHQQAQNQQSQAQSQ | | sequence- |
| | EQESQSAESESAVQSSQSSSGMSSQAQSQQQQAQSQ | | full length |
| | EQESQSCRIRIRCSVKPILQWNEQPSSKPTTTSPEPRTGI | | |
| | PVCRIMSSQAQSQQQQAQVQQSQAQSQEQESQSAGS | | |
| | ESAVQSSKSSSGMSSQAQSQQSQAQSQEQESQSAQS | | |
| | GSDVQVSKSSSGRSSQAQSQQQQSQLQQSQAQSQEQ | | |
| | ESQSAGSESAVQSSKSSSGMSSQAQSQQQQAQVQQS | | |
| | QAQSQEQESQSAQSESAIQSSKSSSGMSSQAQSQQQQ | | |
| | AQVQQSQAQSQEQESQSAQSESAIQSSKSSSGMSSQA | | |
| | QSQQQQAQVQQSQAQSQEQESQSAESESDVQSSKSS | | |
| | RGMSSHAQSQQSQAQSQEQESQSAQSGSDVEVSKSS | | |
| | SGRSSQAQSQQQQSQLQQSQAQSQEQESQSAGSESA | | |
| | VQSSKSSSGMSSQAQSQQQQAQVQQSQAQSQEQESQ | | |
| | SAGSESAVQSSKSSSGMSSQAQSQQQQAQVQQSQAQ | | |
| | SQEQESQSAGSESAVQSSKSSSGMSSQAQSQQQQAQ | | |
| | SQEQESQSAQSESAIQSSKSSSGMSSQAQSQQQQSQL | | |
| | QQSQAQSQEQESQSAESESAVQSSKSSSGMSSQAQSQ | | |
| | QQQAQSQEQESQSAQSESAIQSSKSSSGMSSQAQSQQ | | |
| | QQAQVQQSQAQSQEQESQSAESESDVQSSKSSRGMS | | |
| | SHAQSQQSQAQSQEQESQSAQSGSDVEVSKSSSGRSS | | |
| | QAQSQQQQSQLQQSQAQSQEQESQSAGSESAVQSSK | | |
| | SSSGMSSQAQSQQQQAQVQQSQAQSQEQESQSAGSE | | |
| | SAVQSSKSSSGMSSQAQSQQQQAQSQEQESQSAQSE | | |
| | SAIQSSKSSSGMSSQAQSQQQQSQLQQSQAQSQEQE | | |
| | SQSAESESAVQSSKSSSGMSSQAQSQQQQAQSQEQES | | |
| | QSAQSESAIQSSKSSSGMSSQAQSQQQQAQVQQSQA | | |
| | QSQEQESQSAGSESAVQSSKSSSGMSSQAQSQQQQA | | |
| | QSQEQESQSAQSESAIQSSKSSSGMSSQAQSQQQQSQ | | |
| | LQQSQAQSQEQESQSAESESAVQSSKSSSGMSSQAQS | | |
| | QQQQAQSQEQESQSAQSESAIQSSKSSSGMSSQAQSQ | | |
| | QQQAQVQQSQAQSQEQESQSAESESDVQSSKSSRGM | | |
| | SSHAQSHQSQAQSQEQESQSAQSGSDVEVSKSSSGRS | | |
| | SQAQSQQQQSQLQQSQAQSQEQESQSAGSESAVQSS | | |
| | KSSSGMSSQAQSQQQQAQVQQSQAQSQEQESQANPP | | |
| | VEMSSQAQSQQQQSQLQQSQAQSQEQESQSDQAESD | | |
| | VQSSKSSRGMSSHAQSQQSQAQSQEQESQSAESESAV | | |
| | QSSKSSSGRSSQAQSQQQQSQLQQSQAQSQEQESQS | | |
| | AQSASDVEVSKSSSGRSSQAQSQQQQSQLQQSQAQS | | |
| | QEQESQSAESESDVQSSKSSRGMSSHAQSQQSQAQS | | |
| | QEQESQSAQSGSDVEVSKSSSGRSSQAQSQQQQSQL | | |
| | QQSQAQSQEQESQSAGSESAVQSSKSSRGMSSQAQS | | |
| | QQQQSQLQQSQAQSQEQESQSAQSGSDVEVSKSSSG | | |
| | RSSQAQSQQQQSQLQQSQAQSQEQESQSAESESDVQ | | |
| | SSKSSRGMSSHAQSQQSQAQSQEQESQSAESESAVQS | | |
| | SKSSRGMSSQAQSQQQQSQLQQSQAQSQEQESQSAQ | | |
| | SGSDVEVSKSSSGRSSQAQSQQQQSQLQQSQAQSQE | | |
| | QESQSAGSESAVQSSKSSSGMSSQAQSQQQQSQLQQ | | |
| | SQAQSQEQESQSDQAESDVQSSKSSRGMSSHAQSQQ | | |
| | SQAQSQEQESQSAESESAVQSSKSSSGRSSQAQSQQQ | | |
| | QSQLQQSQAQSQEQESQSAQSGSDVEVSKSSSGRSSQ | | |
| | AQSQQQQSQLQQSQAQSQEQESQSAGSESAVQSSKS | | |
| | SQWNEQPSSKPTTTISAPTVSSSEPRTGIPICSIWIRCML | | |
| | KSSKSSSGRSSQAQSQQQQSQLQQSQAQSQEQESQS | | |
| | AESESDVQSSKSSRGMSSHAQSQQSQAQSQEQESQSA | | |
| | ESESAVQSSKSSRGMSSQAQSQQQQSQLQQSQAQSQ | | |
| | EQESQSAQSGSDVEVSKSSSGRSSQAQSQQQQSQLQ | | |
| | QSQAQSQEQESQSAESESDVQSSKSSRGMSSRAQSQQ | | |
| | SQAQSQEQESQSAQSGSDVEVSKSSSGRSSQAQSQQ | | |
| | QQSQLQQSQAQSQEQESQSAGSESAVQSSKSSQWNE | | |
| | QPSSKPTTTISAPTVPSSEPRTGIPVCRIMSSQAQSQQQ | | |
| | QAQLQQSQAQSQEQESQSAESESDVQSSKSSRGMSSR | | |
| | AQSQQSQAQSQEQESQSAQSGSDVEVSKSSSGRSSQA | | |
| | QSQQQQSQLQQSQAQSQEQESQSAESESDVQSSKSS | | |
| | RGMSSHAQSQQSQAQSQEQESQSAESESAVQSSKSSR | | |
| | GMSSQAQSQQQQSQLQQSQAQSQEQESQSAQSGSDV | | |
| | EVSKSSSGRSSQAQSQQQQSQLQQSQAQSQEQESQS | | |
| | AESESDVQSSKSSRGMSSHAQSQQSQAQSQEQESQSA | | |
| | QSGSDVEVSKSSSGRSSQAQSQQQQSQLQQSQAQSQ | | |
| | EQESQSAESESDVQSSKSSRGMSSRAQSQQSQAQSQE | | |
| | QESQSAQSGSDVDSKQILQWKEQPSSKPTTTISAPTVP | | |
| | SSEPRTGIPVCRIMSSQAQSQQQQSQLQQSQAQSQEQ | | |
| | ESQSAQSGSDVEVSKSSSGRSSQAQSQQQQSQLQQS | | |
| | QAQSQEQESQSAESESAVQSSKSSSGMSSQAQSQQQ | | |
| | QSQLQQSQAQSQEQESQSDQAESDVQSSKSSRGMSS | | |
| | HAQSQQSQAQSQEQESQSAESESAVQSSKSSRGMSSQ | | |
| | AQSQQQQSQLQQSQAQSQEQESQSAQSGSDVEVSKS | | |
| | SSGRSSQAQSQQQQSQLQQSQAQSQEQESQSAESES | | |
| | DVQSSKSSRGMSSRAQSQQSQAQSQEQESQSAQSGS | | |
| | DVDVSKSSSGRSSQAQSQQQQSQLQQSQAQSQEQES | | |
| | QSAGSESAVQSSKSSRGMSSQAQSQQQQSQLQQSQA | | |
| | QSQEQESQSAGSESAVQSSKSSQWNEQPSSKPTTTSS | | |
| | TPTVPSSKPRTGIPVCRIMSSRAQSQQSQAQSQEQESQ | | |
| | SAQSGSDVEVSKSSSGRSSQAQSQQQQSQLQQSQAQ | | |
| | SQEQESQSAESESDVQSSKSSRGMSSHAQSQQSQAQS | | |
| | QEQESQSAESESAVQSSKSSRGMSSQAQSQQQQSQLQ | | |
| | QSQAQSQEQESQSAQSGSDVEVSKSSSGRSSQAQSQ | | |
| | QQQSQLQQSQAQSQEQESQSAESESDVQSSKSSRGM | | |
| | SSHAQSQQSQAQSQEQESQSAQSGSDVEVSKSSSGRS | | |
| | SQAQSQQQQSQLQQSQAQSQEQESQSAGSESAVQSS | | |
| | KSSRGMSSQAQSQQQQSQLQQSQAQSQEQESQSAGS | | |
| | ESAVQSSKSSQWNEQPSSKPTTTSSTPTVPSSEPRTGIP | | |
| | ICSIWIRCMSSHAQSQQSQAQSQEQESQSAESESAVQS | | |
| | SKSSRGMSSQAQSQQQQSQLQQSQAQSQEQESQSAE | | |
| | SGSAVAVKQILQWKEQPSSKPTTTISAPTVPSSEPRTGIP | | |
| | ICSIWIRCMSSHAQSQQSQAQSQEQESQSAESESDVQ | | |
| | SSKSSRGMSSHAQSQQSQAQSQEQESQSAQSGSDVE | | |
| | VSKSSSGRSSQAQSQQQQSQLQQSQAQSQEQESQSA | | |
| | GSESAVQSSKSSRGMSSQAQSQQQQSQLQQSQAQSQ | | |
| | EQESQSDQAESDVQSSKSSRGMSSHAQSQQSQAQSQ | | |
| | EQESQSAESESAVQSSKSSRGMSSQAQSQQQQSQLQQ | | |
| | SQAQSQEQESQSAQSGSDVEVSKSSSGRSSQAQSQQ | | |
| | QQSQLQQSQAQSQEQESQSAQSESDVQSSKSSQWNE | | |
| | QPSSKPTTTSSTPTVPSSKPRTGIPVCRIMSSQAQSQQQ | | |
| | QSQLQQSQAQSQEQESQSAESESAVQSSKSSSGMSSQ | | |
| | AQSQQQQSQLQQSQAQSQEQESQSDQAESDVQSSKS | | |
| | SRGMSSQAQSQQSQAQSQEQESQSAESESAVQSSKSS | | |
| | SGMSSHAQSQQSQAQSQEQESQSAESESAVQSSKSSR | | |
| | GMSSQAQSQQQQSQLQQSQAQSQEQESQSAQSGSDV | | |
| | EVSKSSSGRSSQAQSQQQQSQLQQSQAQSQEQESQS | | |
| | AGSESAVQSSKSSSGMSSQAQSQQQQSQLQQSQAQS | | |
| | QEQESQSAESESDVQSSKSSRGMSSHAQSQQSQAQS | | |
| | QEQESQSAQSGSDVEVSKSSSGRSSQAQSQQQQSQL | | |
| | QQSQAQSQEQESQSAGSESAVQSSKSSQWNEQPSSKP | | |
| | TTTSSTPTVPSSEPRTGIPVCRIMSSHAQSQQSQAQSQE | | |
| | QESQSAESESAVQSSKSSSGMSSQAQSQQQQSQLQQ | | |
| | SQAQSQEQESQSAQSGSDVEVSKSSSGRSSQAQSQQ | | |
| | QQSQLQQSQAQSQEQESQSAGSESAVQSSKSSSGMS | | |
| | SQAQSQQQQSQLQQSQAQSQEQESQSAGSESAVESS | | |
| | KSSRGMSSHAQSQQSQAQSQEQESQSAGSESAVQSS | | |
| | KSSSGMSSQAQSQQQQAQLQQSQAQSQEQESQSAGS | | |
| | ESAVQSSKSSSGMSSQAQSQQQQAQLQQSQAQSQEQ | | |
| | ESQSAESESAVQSSKSSSGMSSQAQSQQQQSQLQQS | | |
| | QAQSQEQESQSAESESAVQSSKSSSGMSSQAQSQQQ | | |
| | QAQLQQSQAQSQEQESQSAGSESAVQSSKSSSGMSS | | |
| | QAQSQQQQAQLQQSQAQSQEQESQSAGSESAVQSSK | | |
| | SSSGMSSQAQSQQQQSQLQQSQAQSQEQESQSAESE | | |
| | SAVESSKSSGMSSHAQSQQSQAQSQEQESQSAGSESA | | |
| | VQSSKSSSGMSSQAQSQQQQAQLQQSQAQSQEQESQ | | |
| | SAGSESAVQSSKSSSGMSSQAQSQQQQAQLQQSQAQ | | |
| | SQEQESQSAESESAVQSSKSSSGMSSQAQSQQQQSQL | | |
| | QQSQAQSQEQESQSAGSESAVQSSKSSSGMSSQAQS | | |
| | QQQQAQLQQSQAQSQEQESQSAGSESAVQSSKSSSG | | |
| | MSSQAQSQQQQSQLQQSQAQSQEQESQSAESESAVE | | |
| | SSKSSRGMSSHAQSQQSQAQSQEQESQSAGSESAVQ | | |
| | SSKSSSGMSSQAQSQQQQAQLQQSQAQSQEQESQSA | | |
| | GSESAVQSSKSSSGMSSQAQSQQSQAQSQEQESQSA | | |
| | QSGSDVQVSKSSSGMSSQAQSQQQQAQVQQSQAQS | | |
| | QEQESQSAQSESAIQSSKSSSGMSSQAQSQQQQAQVQ | | |
| | QSQAQSQEQESQSAQSGSDVQVSKSSSGRSSQAQSQ | | |
| | QQQSQVQQSQAQSQEQESQSAGSESAVQSSKSSSGM | | |
| | SSQAQSQQQQSQLQQSQAQSQEQESQSAESESAVESS | | |
| | KSSRGMSSHAQSQQSQAQSQEQESQSAGSESAVQSS | | |
| | KSSSGMSSQAQSQQQQAQLQQSQAQSQEQESQSAGS | | |
| | ESAVQSSKSSSGMSSQAQSQQQQAQLQQSQAQSQEQ | | |
| | ESQSAGSESAVQSSKSSSGMSSQAQSQQSQAQSQEQE | | |
| | SQSAQSGSDVQVKQILQWNEQPSSKPTTASSSPTVSSS | | |
| | EPRTGIPICSI | | |
| 3 | ATGAGCAGCCAGGCCCAGAGCCAGCAGCAGCAGAGC | NA | Codons |
| | CAGCTGCAGCAGAGCCAGGCCCAGAGCCAGGAGCAG | | specifying |
| | GAGAGCCAGAGCGCCCAGAGCGGCAGCGACGTGGAG | | amino acid |
| | GTGAGCAAGAGCAGCAGCGGC | | residues at |
| | | | positions X1, |
| | | | X2 and X3 of |
| | | | SEQ ID NO: |
| | | | 4 will vary as |
| | | | required to |
| | | | meet the |
| | | | definitions of |
| | | | X1, X2 and |
| | | | X3 disclosed |
| | | | in the |
| | | | specification |
| 4 | MSSQAQSQQQQSQLQQSQAQSQEQESQSAX₁SGSDV | AA | Consensus |
| | X₂X₃SKSSSG | | sequence |
| | | | 43 mer with |
| | | | X1, X2, X3 |
| | | | defined in |
| | | | the |
| | | | specification |
| 5 | AGCGAGAGCGCCGTGCAGAGCAGCAAGAGCAGCAGC | NA | Codons |
| | GGCATGAGCAGCCAGGCCCAGAGCCAGCAGCAGCAG | | specifying |
| | AGCCAGCTGCAGCAGAGCCAGGCCCAGAGCCAGGAG | | amino acid |
| | CAGGAGAGCCAGAGCGCCCAG | | residues at |
| | | | positions X1, |
| | | | X2 of SEQ ID |
| | | | NO: 6 will |
| | | | vary as |
| | | | required to |
| | | | meet the |
| | | | definitions of |
| | | | X1, X2 |
| | | | disclosed in |
| | | | the |
| | | | specification |
| 6 | SESAVQSSKSSSGMSSQAQSQQQQX₁QLQQSQAQSQE | AA | With X1 and |
| | QESQSAX₂ | | X2 defined in |
| | | | the |
| | | | specification; |
| | | | Class A |
| 7 | AGCCAGTGGAACGAGCAGCCCAGCAGCAAGCCCACCA | NA | Linker |
| | CCACCAGCAGCACCCCCACCGTGCCCAGCAGCGAGCC | | peptide |
| | CAGGACCGGCATCCCCATCTGCAGCATCTGGATCAGG | | |
| | TGC | | |
| 8 | SQWNEQPSSKPTTTSSTPTVPSSEPRTGIPICSIWIRC | AA | Linker |
| | | | Peptide |
| 9 | GGATCCATGAAGTGTGTCCTCCTGCTCGTTGTTATTTT | NA | FSS |
| | CGCTGTTGCTGCTTACGCCCGCCCCCAGTCGCACCATC | | (FUNSecSf9), |
| | ACCACCATCACGATTACGACATCCCCACCACTGAGAAC | | codon |
| | CTGTACTTCCAGGGCGCTAAGACCACTCTGAAGTTCCA | | optimised for |
| | CCCACACCACGGATTCAGGAAGCACCACGGTCACCCT | | expression in |
| | AACCACCACAAGGCCAGCAACTCTGAGAAGGAACTGG | | Sf9 cells |
| | AGTGGGACGACAACGTGCAGCAGAAGTACTGGAACGA | | |
| | AAAGCGCCTGCAGTCTGACCGTAAGCACGAATCAGAG | | |
| | TTCGAACAGTCAAAGTCCGAGAACAAGGTCACTAAGTC | | |
| | CAGCTCTACCCACTCATCCCAGCACAACGAAAACCACA | | |
| | CTGAGCTGAGCGAATCTAAGTCAACTCACACCGCTCAC | | |
| | AAGTCCTCCGGTGGCAAGTCCTCCCAGATGGAGTCCC | | |
| | AGCACCAGCAGGCTCAGAACCAGCAGTCCCAGGCCCA | | |
| | GAGCCAGGAGCAGGAATCCCAGAGCGCTGAGTCTGAA | | |
| | TCAGCCGTGCAGTCCTCCCAGTCATCCAGCGGAATGTC | | |
| | TTCACAGGCTCAGTCTCAACAGCAGCAGGCTCAATCTC | | |
| | AAGAACAGGAATCCCAGAGCATGTCCAGCAGGGCTCA | | |
| | GTCTCAGCAGTCACAGGCCCAGTCTCAGGAGCAGGAA | | |
| | TCTCAGTCAGCTCAGTCCGGCAGCGACGTGGAGGTGT | | |
| | CCAAGTCTTCATCCGGAAGAAGCTCTCAGGCCCAGTCC | | |
| | CAGCAGCAGCAGAGCCAGCTGCAGCAGTCTCAGGCTC | | |
| | AGTCACAAGAACAGGAATCTCAGTCAGCCGAGTCCGA | | |
| | AAGCGACGTGCAGTCATCCAAGTCCTCCCGCGGTATG | | |
| | TCATCCCACGCTCAGTCTCAACAGAGCCAGGCTCAGTC | | |
| | CCAAGAACAAGAATCTCAGTCAGCTGAGTCCGAAAGC | | |
| | GCCGTCCAGTCCTCCAAGTCATCCCGTGGCATGTCCTC | | |
| | CCAGGCTCAGTCTCAGCAGCAGCAGTCACAGCTGCAG | | |
| | CAGTCCCAGGCTCAATCTCAAGAGCAAGAATCTCAGTC | | |
| | AGCCCAGTCCGGTAGCGATGTCGAGGTGTCCAAGTCC | | |
| | TCCTCCGGTCGCTCCTCCCAAGCTCAATCTCAACAACA | | |
| | ACAATCTCAATTGCAACAATCACAGGCTCAGTCACAAG | | |
| | AGCAAGAGTCACAATCTGCTGAGTCCGAAAGCGACGT | | |
| | CCAGTCCAGCAAGTCTTCAAGGGGAATGTCCAGCCAC | | |
| | GCTCAATCACAACAATCACAGGCCCAGTCCCAAGAACA | | |
| | GGAGTCACAATCAGCTCAGTCCGGAAGCGACGTGGAG | | |
| | GTCAGCAAGTCTTCATCCGGTAGAAGCTCTCAAGCCCA | | |
| | ATCACAACAACAACAATCTCAGTTGCAACAATCTCAAG | | |
| | CTCAAAGCCAAGAACAGGAGTCTCAGTCAGCTGGCTC | | |
| | CGAAAGCGCTGTCCAGTCATCCAAGTCCTCCCGCGGT | | |
| | ATGTCATCCCAGGCTCAATCTCAGCAACAACAATCTCA | | |
| | ACTCCAGCAGTCTCAGGCCCAGTCACAAGAGCAAGAA | | |
| | TCACAGTCAGCTGGTTCCGAGTCTGCTGTGCAGTCCTC | | |
| | CAAGTCATCCCAGTGGAACGAACAGCCTAGCTCTAAGC | | |
| | CCACCACTACCTCATCCACTCCTACCGTTCCGAGTTCT | | |
| | GAGCCCCGCACAGGTATTCCGATTTGTAGTATTTGGAT | | |
| | TCGTTGCTAAGGATCC | | |
| 10 | MKCVLLLVVIFAVAAYARPQSHHHHHHDYDIPTTENLYF | AA | FSS |
| | QGAKTTLKFHPHHGFRKHHGHPNHHKASNSEKELEWD | | (FUNSecSf9) |
| | DNVQQKYWNEKRLQSDRKHESEFEQSKSENKVTKSSS | | |
| | THSSQHNENHTELSESKSTHTAHKSSGGKSSQMESQH | | |
| | QQAQNQQSQAQSQEQESQSAESESAVQSSQSSSGMS | | |
| | SQAQSQQQQAQSQEQESQSSKSSRGMSSRAQSQQSQ | | |
| | AQSQEQESQSAQSGSDVEVSKSSSGRSSQAQSQQQQ | | |
| | SQLQQSQAQSQEQESQSAESESDVQSSKSSRGMSSHA | | |
| | QSQQSQAQSQEQESQSAESESAVQSSKSSRGMSSQA | | |
| | QSQQQQSQLQQSQAQSQEQESQSAQSGSDVEVSKSS | | |
| | SGRSSQAQSQQQQSQLQQSQAQSQEQESQSAESESD | | |
| | VQSSKSSRGMSSHAQSQQSQAQSQEQESQSAQSGSD | | |
| | VEVSKSSSGRSSQAQSQQQQSQLQQSQAQSQEQESQ | | |
| | SAGSESAVQSSKSSRGMSSQAQSQQQQSQLQQSQAQ | | |
| | SQEQESQSAGSESAVQSSKSSQWNEQPSSKPTTTSSTP | | |
| | TVPSSEPRTGIPICSIWIRC | | |
| 11 | CGCCCCCAGTCGCACCATCACCACCATCACGATTACGA | NA | FSS |
| | CATCCCCACCACTGAGAACCTGTACTTCCAGGGCGCTA | | (FUNSecSf9) |
| | AGACCACTCTGAAGTTCCACCCACACCACGGATTCAGG | | with |
| | AAGCACCACGGTCACCCTAACCACCACAAGGCCAGCA | | secretion tag |
| | ACTCTGAGAAGGAACTGGAGTGGGACGACAACGTGCA | | cleaved |
| | GCAGAAGTACTGGAACGAAAAGCGCCTGCAGTCTGAC | | |
| | CGTAAGCACGAATCAGAGTTCGAACAGTCAAAGTCCG | | |
| | AGAACAAGGTCACTAAGTCCAGCTCTACCCACTCATCC | | |
| | CAGCACAACGAAAACCACACTGAGCTGAGCGAATCTA | | |
| | AGTCAACTCACACCGCTCACAAGTCCTCCGGTGGCAA | | |
| | GTCCTCCCAGATGGAGTCCCAGCACCAGCAGGCTCAG | | |
| | AACCAGCAGTCCCAGGCCCAGAGCCAGGAGCAGGAAT | | |
| | CCCAGAGCGCTGAGTCTGAATCAGCCGTGCAGTCCTC | | |
| | CCAGTCATCCAGCGGAATGTCTTCACAGGCTCAGTCTC | | |
| | AACAGCAGCAGGCTCAATCTCAAGAACAGGAATCCCA | | |
| | GAGCATGTCCAGCAGGGCTCAGTCTCAGCAGTCACAG | | |
| | GCCCAGTCTCAGGAGCAGGAATCTCAGTCAGCTCAGT | | |
| | CCGGCAGCGACGTGGAGGTGTCCAAGTCTTCATCCGG | | |
| | AAGAAGCTCTCAGGCCCAGTCCCAGCAGCAGCAGAGC | | |
| | CAGCTGCAGCAGTCTCAGGCTCAGTCACAAGAACAGG | | |
| | AATCTCAGTCAGCCGAGTCCGAAAGCGACGTGCAGTC | | |
| | ATCCAAGTCCTCCCGCGGTATGTCATCCCACGCTCAGT | | |
| | CTCAACAGAGCCAGGCTCAGTCCCAAGAACAAGAATCT | | |
| | CAGTCAGCTGAGTCCGAAAGCGCCGTCCAGTCCTCCA | | |
| | AGTCATCCCGTGGCATGTCCTCCCAGGCTCAGTCTCAG | | |
| | CAGCAGCAGTCACAGCTGCAGCAGTCCCAGGCTCAAT | | |
| | CTCAAGAGCAAGAATCTCAGTCAGCCCAGTCCGGTAG | | |
| | CGATGTCGAGGTGTCCAAGTCCTCCTCCGGTCGCTCCT | | |
| | CCCAAGCTCAATCTCAACAACAACAATCTCAATTGCAA | | |
| | CAATCACAGGCTCAGTCACAAGAGCAAGAGTCACAATC | | |
| | TGCTGAGTCCGAAAGCGACGTCCAGTCCAGCAAGTCT | | |
| | TCAAGGGGAATGTCCAGCCACGCTCAATCACAACAATC | | |
| | ACAGGCCCAGTCCCAAGAACAGGAGTCACAATCAGCT | | |
| | CAGTCCGGAAGCGACGTGGAGGTCAGCAAGTCTTCAT | | |
| | CCGGTAGAAGCTCTCAAGCCCAATCACAACAACAACAA | | |
| | TCTCAGTTGCAACAATCTCAAGCTCAAAGCCAAGAACA | | |
| | GGAGTCTCAGTCAGCTGGCTCCGAAAGCGCTGTCCAG | | |
| | TCATCCAAGTCCTCCCGCGGTATGTCATCCCAGGCTCA | | |
| | ATCTCAGCAACAACAATCTCAACTCCAGCAGTCTCAGG | | |
| | CCCAGTCACAAGAGCAAGAATCACAGTCAGCTGGTTCC | | |
| | GAGTCTGCTGTGCAGTCCTCCAAGTCATCCCAGTGGAA | | |
| | CGAACAGCCTAGCTCTAAGCCCACCACTACCTCATCCA | | |
| | CTCCTACCGTTCCGAGTTCTGAGCCCCGCACAGGTATT | | |
| | CCGATTTGTAGTATTTGGATTCGTTGCTAAGGATCC | | |
| 12 | RPQSHHHHHHDYDIPTTENLYFQGAKTTLKFHPHHGFRK | AA | FSS |
| | HHGHPNHHKASNSEKELEWDDNVQQKYWNEKRLQSD | | (FUNSecSf9) |
| | RKHESEFEQSKSENKVTKSSSTHSSQHNENHTELSESK | | with |
| | STHTAHKSSGGKSSQMESQHQQAQNQQSQAQSQEQE | | secretion tag |
| | SQSAESESAVQSSQSSSGMSSQAQSQQQQAQSQEQE | | cleaved |
| | SQSMSSRAQSQQSQAQSQEQESQSAQSGSDVEVSKS | | |
| | SSGRSSQAQSQQQQSQLQQSQAQSQEQESQSAESES | | |
| | DVQSSKSSRGMSSHAQSQQSQAQSQEQESQSAESESA | | |
| | VQSSKSSRGMSSQAQSQQQQSQLQQSQAQSQEQESQ | | |
| | SAQSGSDVEVSKSSSGRSSQAQSQQQQSQLQQSQAQ | | |
| | SQEQESQSAESESDVQSSKSSRGMSSHAQSQQSQAQS | | |
| | QEQESQSAQSGSDVEVSKSSSGRSSQAQSQQQQSQL | | |
| | QQSQAQSQEQESQSAGSESAVQSSKSSRGMSSQAQS | | |
| | QQQQSQLQQSQAQSQEQESQSAGSESAVQSSKSSQW | | |
| | NEQPSSKPTTTSSTPTVPSSEPRTGIPICSIWIRC | | |
| 13 | ATGTCTTCACGTGCTCAGTCACAGCAGTCCCAGGCCCA | NA | Module 7, |
| | GAGCCAGGAGCAGGAATCACAGTCCGCTCAGAGCGGT | | codon |
| | TCTGACGTGGAGGTGTCCAAGTCCAGCTCTGGCAGGT | | optimised for |
| | CATCCCAGGCCCAGTCACAGCAGCAGCAGTCCCAGCT | | Sf9 |
| | GCAGCAGAGCCAGGCTCAGTCTCAAGAACAGGAATCA | | expression |
| | CAGTCCGCCGAGAGCGAATCTGACGTGCAGTCCTCCA | | |
| | AGTCATCCAGGGGAATGTCCTCCCACGCTCAGAGCCA | | |
| | GCAGTCTCAGGCCCAGTCTCAAGAGCAAGAGTCACAG | | |
| | TCCGCTGAGAGCGAATCTGCCGTCCAGTCATCCAAGTC | | |
| | CTCCCGCGGTATGTCATCCCAGGCTCAGAGCCAGCAG | | |
| | CAGCAGTCTCAGCTGCAGCAGTCACAGGCTCAGTCTC | | |
| | AAGAGCAAGAATCTCAGTCCGCCCAGAGCGGATCTGA | | |
| | TGTCGAGGTGTCCAAGTCCTCCTCCGGTCGCTCCTCCC | | |
| | AGGCTCAATCTCAACAACAACAATCTCAATTGCAACAA | | |
| | TCTCAGGCTCAGTCTCAGGAACAAGAGTCTCAATCTGC | | |
| | TGAATCTGAGTCAGACGTGCAGTCTTCAAAGTCCAGCC | | |
| | GCGGCATGTCTTCACACGCTCAATCTCAACAATCCCAG | | |
| | GCCCAGTCTCAGGAACAAGAATCTCAGTCCGCTCAGA | | |
| | GCGGCTCTGACGTGGAGGTCAGCAAGTCCAGCTCTGG | | |
| | ACGTTCATCCCAAGCCCAATCTCAACAACAACAGTCAC | | |
| | AATTGCAGCAGTCACAGGCCCAGTCTCAGGAGCAAGA | | |
| | ATCACAGTCCGCTGGTAGCGAATCTGCTGTCCAGTCCT | | |
| | CCAAGTCATCCAGAGGCATGTCCTCCCAGGCTCAATCC | | |
| | CAACAACAACAATCTCAGTTGCAGCAGAGTCAAGCCCA | | |
| | GTCTCAAGAGCAGGAGTCACAGTCCGCTGGAAGCGAG | | |
| | TCAGCTGTGCAGTCATCCAAGTCCTCCCAGTGGAACGA | | |
| | ACAGCCTTCATCCAAGCCCACCACTACCAGCTCTACTC | | |
| | CTACCGTCCCATCTTCGGAGCCACGCACGGGCATTCCT | | |
| | ATCTGTTCTATTTGGATTCGCTGCTAA | | |
| 14 | MSSRAQSQQSQAQSQEQESQSAQSGSDVEVSKSSSG | AA | Module 7 |
| | RSSQAQSQQQQSQLQQSQAQSQEQESQSAESESDVQ | | |
| | SSKSSRGMSSHAQSQQSQAQSQEQESQSAESESAVQS | | |
| | SKSSRGMSSQAQSQQQQSQLQQSQAQSQEQESQSAQ | | |
| | SGSDVEVSKSSSGRSSQAQSQQQQSQLQQSQAQSQE | | |
| | QESQSAESESDVQSSKSSRGMSSHAQSQQSQAQSQE | | |
| | QESQSAQSGSDVEVSKSSSGRSSQAQSQQQQSQLQQ | | |
| | SQAQSQEQESQSAGSESAVQSSKSSRGMSSQAQSQQ | | |
| | QQSQLQQSQAQSQEQESQSAGSESAVQSSKSSQWNE | | |
| | QPSSKPTTTSSTPTVPSSEPRTGIPICSIWIRC | | |
| 15 | ATGAGCAGCCAGGCCCAGAGCCAGCAGCAGCAGAGC | NA | The majority |
| | CAGCTGCAGCAGAGCCAGGCCCAGAGCCAGGAGCAG | | rule |
| | GAGAGCCAGAGCGCCGAGAGCGAGAGCGACGTGCAG | | consensus |
| | AGCAGCAAGAGCAGCAGCGGCAGGAGCAGCCAGGCC | | sequence on |
| | CAGAGCCAGCAGAGCCAGGCCCAGAGCCAGGAGCAG | | page 9 of the |
| | GAGAGCCAGAGCGCCCAGAGCGAGAGCGACGTGGAG | | module |
| | GTGAGCAAGAGCAGCAGCGGCAGGAGCAGCCAGGCC | | consensus |
| | CAGAGCCAGCAGCAGCAGAGCCAGCTGCAGCAGAGC | | |
| | CAGGCCCAGAGCCAGGAGCAGGAGAGCCAGAGCGCC | | |
| | GAGAGCGAGAGCGACGTGCAGAGCAGCAAGAGCAGC | | |
| | AGGGGCATGAGCAGCCACGCCCAGAGCCAGCAGAGC | | |
| | CAGGCCCAGAGCCAGGAGCAGGAGAGCCAGAGCGCC | | |
| | GAGAGCGAGAGCGCCGTGCAGAGCAGCAAGAGCAGC | | |
| | AGGGGCATGAGCAGCCAGGCCCAGAGCCAGCAGCAG | | |
| | CAGAGCCAGCTGCAGCAGAGCCAGGCCCAGAGCCAG | | |
| | GAGCAGGAGAGCCAGAGCGCCCAGAGCGGCAGCGAC | | |
| | GTGGAGGTGAGCAAGAGCAGCAGCGGCAGGAGCAGC | | |
| | CAGGCCCAGAGCCAGCAGCAGCAGAGCCAGCTGCAG | | |
| | CAGAGCCAGGCCCAGAGCCAGGAGCAGGAGAGCCAG | | |
| | AGCGCCGAGAGCGAGAGCGACGTGCAGAGCAGCAAG | | |
| | AGCAGCAGGGGCATGAGCAGCAGGGCCCAGAGCCAG | | |
| | CAGAGCCAGGCCCAGAGCCAGGAGCAGGAGAGCCAG | | |
| | AGCGCCCAGAGCGGCAGCGACGTGGAGGTGAGCAAG | | |
| | AGCAGCAGCGGCAGGAGCAGCCAGGCCCAGAGCCAG | | |
| | CAGCAGCAGAGCCAGCTGCAGCAGAGCCAGGCCCAG | | |
| | AGCCAGGAGCAGGAGAGCCAGAGCGCCCAGAGCGAG | | |
| | AGCGACGTGGAGGTGAGCAAGAGCAGCAGCGGCAGG | | |
| | AGCAGCCAGGCCCAGAGCCAGCAGCAGCAGAGCCAG | | |
| | CTGCAGCAGAGCCAGGCCCAGAGCCAGGAGCAGGAG | | |
| | AGCCAGAGCGCCGGCAGCGAGAGCGCCGTGCAGAGC | | |
| | AGCAAGAGCAGCCAGTGGAACGAGCAGCCCAGCAGCA | | |
| | AGCCCACCACCACCATCAGCGCCCCCACCGTGCCCAG | | |
| | CAGCGAGCCCAGGACCGGCATCCCCGTGTGCAGGATC | | |
| 16 | MSSQAQSQQQQSQLQQSQAQSQEQESQSAESESDVQ | AA | The majority |
| | SSKSSSGRSSQAQSQQSQAQSQEQESQSAQSESDVEV | | rule |
| | SKSSSGRSSQAQSQQQQSQLQQSQAQSQEQESQSAE | | consensus |
| | SESDVQSSKSSRGMSSHAQSQQSQAQSQEQESQSAES | | sequence of |
| | ESAVQSSKSSRGMSSQAQSQQQQSQLQQSQAQSQEQ | | the module |
| | ESQSAQSGSDVEVSKSSSGRSSQAQSQQQQSQLQQS | | 3-10 |
| | QAQSQEQESQSAESESDVQSSKSSRGMSSRAQSQQSQ | | alignment |
| | AQSQEQESQSAQSGSDVEVSKSSSGRSSQAQSQQQQ | | |
| | SQLQQSQAQSQEQESQSAQSESDVEVSKSSSGRSSQA | | |
| | QSQQQQSQLQQSQAQSQEQESQSAGSESAVQSSKSS | | |
| | QWNEQPSSKPTTTISAPTVPSSEPRTGIPVCRI | | |
| 17 | AGCGAGAGCGCCGTGCAGAGCAGCAAGAGCAGCAGC | NA | Class B |
| | GGCATGAGCAGCCAGGCCCAGAGCCAGCAGCAGCAG | | |
| | GCCCAGAGCCAGGAGCAGGAGAGCCAGAGCGCCCAG | | |
| 18 | SESAVQSSKSSSGMSSQAQSQQQQAQSQEQESQSAQ | AA | Class B |
| 19 | AGCGAGAGCGACGTGCAGAGCAGCAAGAGCAGCAGG | NA | Class C |
| | GGCATGAGCAGCCACGCCCAGAGCCAGCAGAGCCAG | | |
| | GCCCAGAGCCAGGAGCAGGAGAGCCAGAGCGCCCAG | | |
| 20 | SESDVQSSKSSRGMSSHAQSQQSQAQSQEQESQSAQ | AA | Class C |
| 21 | ATGAGCAGCAGGGCCCAGAGCCAGCAGAGCCAGGCC | NA | Class D |
| | CAGAGCCAGGAGCAGGAGAGCCAGAGCGCCCAG | | |
| 22 | MSSRAQSQQSQAQSQEQESQSAQ | AA | Class D |
| 23 | AGCGAGAGCGCCGTGCAGAGCAGCAAGAGCAGC | NA | Class E |
| 24 | SESAVQSSKSS | AA | Class E |
| 25 | TACTCATTCAGTGCAAAGATGAAATGTGTCTTGTTATTA | NA | the genomic |
| | GTTGTCATATTCGCAGTCGCGGCTTATGCCCGACCTCA | | sequence |
| | GTCGCCACACCATGGTCACCATGGTGCGAAGACTACTT | | (20,870 bp) |
| | TGAAATTCCATCCCCACCATGGATTCCGCAAACATCAC | | H. nubilosus |
| | GGCCATCCTAATCATCATAAAATGGTAAGTCTTTATTTG | | gene that |
| | AGACATACTTTAAAGTGTCCACCATATATACATTCAAAA | | encodes a |
| | CGGAGCTAAACGTATGCTGCAAGGCTTATGTAGCACG | | nest material |
| | TATGTCAACTATGATCTTAAATGATTTTAGTCTTATACA | | polypeptide |
| | ATCTATATTTACCAATAAAACATCATTTCAACTTTCAAA | | (designated |
| | CGTAATTGGGTCGTAGTCACACCGAGTATACAGTACTC | | FUN_069765-T1). |
| | GACGCTACTTTAGCATCACTGTCCATCAAGTIGTCATA | | |
| | AACTTATCACTATCACCTAACTGTGGGCATTATCAACAA | | |
| | ATGTTATGTGTTCGATGATGACCATGCAGCTAACCAGT | | |
| | AACAAAAGCAAGCAGAAACAAAGTTTTAGATTCTACAT | | |
| | CTTGTCATTATAACTGTACAATTAAAACTAATTGATTAT | | |
| | ATTTATACCGAACAAATAATATTAAACATAATCATATTT | | |
| | GAACAATTTGGAATAAGAACATAAGCAATTATACATAA | | |
| | AAACGATTAGATACCACAATTTTCAATATAACGAATAAC | | |
| | TCCTCATCATAACAATCAATCGGTTATTTCTTGACAAAT | | |
| | AATTATATTTAACAAACCAATGTAAAAAATAAAGAAGAA | | |
| | AATAGATTCATAATCTCTACAGTCGTGCCATTATACGTT | | |
| | ATATATTAGGTCACGATGCTATTCCCCCAAAAATAATG | | |
| | AATTATATCCAAGTCAAAAAAATACAAAATTGAAAATTA | | |
| | GGATGGACGCAAAAGCATTACACTGCTATAACATCTAC | | |
| | TACGGTGGGTTTGCAGGCTTCCAACAGCGAAAAAGAA | | |
| | CTAGAATGGGATGACAACGTACAGCAGAAATACTGGA | | |
| | AT | | |
| | GAGAAACGACTCCAATCCGATAGAAAACATGAATCAGA | | |
| | ATTCGAACAATCCAAATCCGAAAACAAAGTAACAAAAT | | |
| | CGTCCAGTACACACTCTAGCCAACATAATGAGAACCAT | | |
| | ACGGAACTATCTGAGTCAAAATCCACACATACGGCACA | | |
| | TAAATCGTCAGGAGGAAAATCAAGCCAAATGGAAAGC | | |
| | CAACACCAACAAGCTCAAAACCAACAGTCCCAAGCCCA | | |
| | GAGCCAAGAACAGGAATCCCAGTCTGCAGAATCTGAA | | |
| | TCCGCTGTTCAGTCAAGCCAATCCTCCAGTGGAATGAG | | |
| | CAGCCAAGCTCAAAGCCAACAACAACAAGCCCAGAGC | | |
| | CAAGAACAGGAATCCCAGTCCTGCAGAATCAGAATCC | | |
| | GCTGTTCAGTCAAGCCAATCCTCCAGTGGAATGAGCA | | |
| | GCCAAGCTCAAAGCCAACAACAACAAGCCCAGAGCCA | | |
| | AGAACAGGAATCCCAGTCTGCAGAATCTGAATCCGCTG | | |
| | TTCAGTCAAGCAAATCCTCCAGTGGAATGAGCAGCCAA | | |
| | GCTCAAAGCCAACAACAGCAAGCTCAAGTCCAACAGTC | | |
| | TCAAGCTCAGAGCCAAGAACAGGAGTCCCAGTCTGCA | | |
| | GGATCTGAATCCGCCGTTCAGTCAAGCAAATCCTCCAG | | |
| | TGGAATGAGCAGCCAAGCTCAAAGCCAACAGTCCCAA | | |
| | GCTCAGAGCCAAGAACAGGAATCCCAATCTGCTCAATC | | |
| | TGGATCCGATGTTCAAGTAAGCAAATCCTCCAGTGGAA | | |
| | GGAGCAGCCAAGCTCAAAGCCAACAACAACAATCTCA | | |
| | GCTCCAACAGTCCCAAGCTCAGAGCCAAGAACAGGAA | | |
| | TCCCAGTCTGCAGGATCTGAATCCGCCGTTCAGTCAAG | | |
| | CAAATCCTCCAGTGGAATGAGCAGCCAAGCTCAAAGC | | |
| | CAACAACAGCAAGCTCAAGTCCAACAGTCTCAAGCTCA | | |
| | GAGCCAAGAACAGGAATCCCAATCTGCTCAATCTGAAT | | |
| | CCGCCATTCAGTCAAGCAAATCC | | |
| | TCCAGTGGAATGAGCAGCCAAGCTCAAAGCCAACAAC | | |
| | AGCAAGCTCAAGTCCAACAGTCTCAAGCTCAGAGCCAA | | |
| | GAACAGGAATCCCAATCTGCTCAATCTGAATCCGCCAT | | |
| | TCAGTCAAGCAAATCCTCCAGTGGAATGAGCAGCCAA | | |
| | GCTCAAAGCCAACAACAGCAAGCTCAAGTCCAACAGTC | | |
| | CCAAGCTCAAAGCCAAGAACAGGAATCCCAGTCTGCA | | |
| | GAATCTGAATCCGATGTTCAGTCAAGCAAATCCTCCAG | | |
| | GGGAATGAGCAGCCATGCTCAAAGCCAACAGTCCCAA | | |
| | GCTCAGAGCCAAGAACAGGAATCCCAATCTGCTCAATC | | |
| | TGGATCCGATGTTGAAGTAAGCAAATCCTCCAGTGGAA | | |
| | GGAGCAGCCAAGCTCAAAGCCAACAACAACAATCTCA | | |
| | GCTCCAACAGTCCCAAGCTCAGAGCCAAGAACAGGAA | | |
| | TCCCAGTCTGCAGGATCTGAATCCGCCGTTCAGTCAAG | | |
| | CAAATCCTCCAGTGGAATGAGCAGCCAAGCTCAAAGC | | |
| | CAACAACAGCAAGCTCAAGTCCAACAGTCTCAAGCTCA | | |
| | GAGCCAAGAACAGGAATCCCAGTCTGCAGGATCTGAA | | |
| | TCCGCCGTTCAGTCAAGCAAATCCTCCAGTGGAATGAG | | |
| | CAGCCAAGCTCAAAGCCAACAACAGCAAGCTCAAGTC | | |
| | CAACAGTCTCAAGCTCAGAGCCAAGAACAGGAGTCAC | | |
| | AGTCTGCAGGATCTGAATCCGCCGTTCAGTCAAGCAAA | | |
| | TCCTCCAGTGGAATGAGCAGCCAAGCTCAAAGCCAAC | | |
| | AACAACAAGCTCAGAGCCAAGAACAGGAATCCCAATCT | | |
| | GCTCAATCTGAATCCGCCATTCAGTCAAGCAAATCCTC | | |
| | CAGTGGAATGAGTAGCCAAGCTCAAAGCCAACAACAA | | |
| | CAATCTCAGCTCCAACAGTCCCAAGCTCAGAGCCAAGA | | |
| | ACAGGAATCCCAGTCTGCAGAATCTGAATCCGCCGTTC | | |
| | AGTCAAGCAAATCCTCCAGTGGAA | | |
| | TGAGCAGCCAAGCTCAAAGCCAACAACAACAAGCTCA | | |
| | GAGCCAAGAACAGGAATCGCAATCTGCTCAATCTGAAT | | |
| | CCGCCATTCAGTCAAGCAAATCCTCCAGTGGAATGAGC | | |
| | AGCCAAGCTCAAAGCCAACAACAGCAAGCTCAAGTCC | | |
| | AACAGTCCCAAGCTCAAAGCCAAGAACAGGAATCCCA | | |
| | GTCTGCAGAATCTGAATCCGATGTTCAGTCAAGCAAAT | | |
| | CCTCCAGGGGAATGAGCAGCCATGCTCAAAGCCAACA | | |
| | GTCCCAAGCTCAGAGCCAAGAACAGGAATCCCAATCT | | |
| | GCTCAATCTGGATCCGATGTTGAAGTAAGCAAATCCTC | | |
| | CAGTGGAAGGAGCAGCCAAGCTCAAAGCCAACAACAA | | |
| | CAATCTCAGCTCCAACAGTCCCAAGCTCAGAGCCAAGA | | |
| | ACAGGAATCCCAGTCTGCAGGATCTGAATCCGCCGTTC | | |
| | AGTCAAGCAAATCCTCCAGTGGAATGAGCAGCCAAGC | | |
| | TCAAAGCCAACAACAGCAAGCTCAAGTCCAACAGTCTC | | |
| | AAGCTCAGAGCCAAGAACAGGAGTCCCAGTCTGCAGG | | |
| | ATCTGAATCCGCCGTTCAGTCAAGCAAATCCTCCAGTG | | |
| | GAATGAGCAGCCAAGCTCAAAGCCAACAACAACAAGC | | |
| | TCAGAGCCAAGAACAGGAATCCCAATCTGCTCAATCTG | | |
| | AATCCGCCATTCAGTCAAGCAAATCCTCCAGTGGAATG | | |
| | AGCAGCCAAGCTCAAAGCCAACAACAACAATCTCAGCT | | |
| | CCAACAGTCCCAAGCTCAGAGCCAAGAACAGGAATCC | | |
| | CAGTCTGCAGAATCTGAATCCGCTGTTCAGTCAAGCAA | | |
| | ATCCTCCAGTGGAATGAGCAGCCAAGCTCAAAGCCAA | | |
| | CAACAACAAGCTCAGAGCCAAGAACAGGAATCCCAAT | | |
| | CTGCTCAATCTGAATCCGCCATTCAGTCAAGCAAATCC | | |
| | TCCAGTGGAATGAGCAGCCAAGCTCAAAGCCAACAAC | | |
| | AGCAAGCTCAAGTCCAACAGTCTCA | | |
| | AGCTCAGAGCCAAGAACAGGAGTCCCAGTCTGCAGGA | | |
| | TCTGAATCCGCCGTTCAGTCAAGCAAATCCTCCAGTGG | | |
| | AATGAGCAGCCAAGCTCAAAGCCAACAACAACAAGCT | | |
| | CAGAGCCAAGAACAGGAATCCCAATCTGCTCAATCTGA | | |
| | ATCCGCCATTCAGTCAAGCAAATCCTCCAGTGGAATGA | | |
| | GTAGCCAAGCTCAAAGCCAACAACAACAATCTCAGCTC | | |
| | CAACAGTCCCAAGCTCAGAGCCAAGAACAGGAATCCC | | |
| | AGTCTGCAGAATCTGAATCCGCTGTTCAGTCAAGCAAA | | |
| | TCCTCCAGTGGAATGAGCAGCCAAGCTCAAAGCCAAC | | |
| | AACAACAAGCTCAGAGCCAAGAACAGGAATCGCAATC | | |
| | TGCTCAATCTGAATCCGCCATTCAGTCAAGCAAATCCT | | |
| | CCAGTGGAATGAGCAGCCAAGCTCAAAGCCAACAACA | | |
| | GCAAGCTCAAGTCCAACAGTCCCAAGCTCAAAGCCAA | | |
| | GAACAGGAATCCCAGTCTGCAGAATCTGAATCCGATGT | | |
| | TCAGTCAAGCAAATCCTCCAGGGGAATGAGCAGCCAT | | |
| | GCTCAAAGCCACCAGTCCCAAGCTCAGAGCCAAGAAC | | |
| | AGGAATCCCAATCTGCTCAATCTGGATCCGATGTTGAA | | |
| | GTAAGCAAATCCTCCAGTGGAAGGAGCAGCCAAGCTC | | |
| | AAAGCCAACAACAACAATCTCAGCTCCAACAGTCCCAA | | |
| | GCTCAGAGCCAAGAACAGGAATCCCAGTCTGCAGGAT | | |
| | CTGAATCCGCCGTTCAGTCAAGCAAATCCTCCAGTGGA | | |
| | ATGAGCAGCCAAGCTCAAAGCCAACAACAACAAGCTC | | |
| | AAGTCCAACAGTCTCAAGCTCAGAGCCAAGAACAGGA | | |
| | GTCCCAGTCTGCAGGATCTGAATCCGCCGTTCAGTCAA | | |
| | GCAAATCCTCCAGTGGAATGAGCAGCCAAGCTCAAAG | | |
| | CCAACAATCAATCAAGCTCAGAGCCAAGAACAGGAATC | | |
| | CCAATCTGCTCAATCTGAATCCGCCA | | |
| | TTCAGTCAAGCAAATCCTCCAGTGGAATGAGCAGCCAA | | |
| | GCTCAAAGCCAACAACAGCAAGCTCAAGCTCCAACAGT | | |
| | CTCAAGCTCAGAGCCAAGAACAGGAATCCCAATCTGCT | | |
| | CAATCTGGATCCGACGTTCAAGTAAGCAAATCCTCCAG | | |
| | TGGAAGGAGCAGCCAAGCTCAAAGCCAACAACAACAA | | |
| | TCTCAGCTCCAACAGTCCCAAGCTCAGAGCCAAGAACA | | |
| | GGAATCCCAGTCTGCAGGATCTGAATCCGCCGTTCAGT | | |
| | CAAGCAAATCCTCTCAGTGGAATGAGCAGCCAAGCTCA | | |
| | AAGCCAACAACAGCAATCTCAACTGCAACAGTCCCAAG | | |
| | CTCAGAGCCAAGAACAGGAATCTCAATCCGACCAGGC | | |
| | TGAATCCGATGTTCAGTCAAGCAAATCCTCCAGGGGAA | | |
| | TGAGCAGCCACGCTCAAAGCCAACAGTCTCAAGCTCAA | | |
| | AGCCAGGAACAGGAATCCCAGTCAGCAGAATCTGAGT | | |
| | CCGCTGTTCAGTCAAGCAAATCCTCCAGTGGAAGGAG | | |
| | CAGCCAAGCTCAAAGCCAACAACAACAATCTCAGCTCC | | |
| | AACAGTCCCAAGCTCAGAGCCAAGAACAGGAATCCCA | | |
| | ATCTGCTCAATCTGCATCCGATGTTGAAGTAAGCAAAT | | |
| | CCTCCAGTGGAAGGAGCAGCCAAGCTCAAAGCCAACA | | |
| | ACAACAATCTCAGCTCCAACAGTCCCAAGCTCAAAGCC | | |
| | AAGAACAGGAATCCCAGTCTGCAGAATCTGAATCCGAT | | |
| | GTTCAGTCAAGCAAATCCTCCAGGGGAATGAGCAGCC | | |
| | ATGCTCAAAGCCAACAGTCCCAAGCTCAGAGCCAAGA | | |
| | ACAGGAATCCCAATCTGCTCAATCTGGATCCGATGTTG | | |
| | AAGTAAGCAAATCCTCCAGTGGAAGGAGCAGCCAAGC | | |
| | TCAAAGCCAACAACAACAATCTCAGCTCCAACAGTCCC | | |
| | AAGCTCAGAGCCAAGAACAGGAATCCCAGTCTGCAGG | | |
| | ATCTGAATCCGCCGTTCAGTCA | | |
| | AGCAAATCCTCTCGTGGAATGAGCAGCCAAGCTCAAA | | |
| | GCCAACAACAACAATCTCAGCTCCAACAGTCTCAAGCT | | |
| | CAGAGCCAAGAACAGGAATCCCAATCTGCTCAATCTGG | | |
| | ATCCGATGTTGAAGTAAGCAAATCCTCCAGTGGAAGGA | | |
| | GCAGCCAAGCTCAAAGCCAACAACAACAATCTCAGCTC | | |
| | CAACAGTCCCAAGCTCAAAGCCAAGAACAGGAATCCC | | |
| | AGTCTGCAGAATCTGAATCCGATGTTCAGTCTAGCAAA | | |
| | TCCTCCAGGGGAATGAGCAGCCATGCTCAAAGCCAAC | | |
| | AGTCTCAAGCTCAAAGCCAGGAACAGGAATCCCAGTC | | |
| | AGCAGAATCTGAATCCGCCGTTCAGTCAAGCAAATCCT | | |
| | CTCGTGGAATGAGCAGCCAAGCTCAAAGCCAACAACA | | |
| | ACAATCTCAGCTCCAACAGTCTCAAGCTCAGAGCCAAG | | |
| | AACAGGAATCCCAATCTGCTCAATCTGGATCCGATGTT | | |
| | GAAGTAAGCAAATCCTCCAGTGGAAGGAGCAGCCAAG | | |
| | CTCAAAGCCAACAACAACAATCTCAGCTCCAACAGTCC | | |
| | CAAGCTCAGAGCCAAGAACAGGAATCCCAGTCTGCAG | | |
| | GATCTGAATCCGCCGTTCAGTCAAGCAAATCCTCCAGT | | |
| | GGAATGAGCAGCCAAGCTCAAAGCCAACAACAGCAAT | | |
| | CTCAACTCCAACAGTCCCAAGCTCAGAGCCAAGAACAG | | |
| | GAATCTCAATCCGACCAGGCTGAATCCGATGTTCAGTC | | |
| | AAGCAAATCCTCCAGGGGAATGAGCAGCCACGCTCAA | | |
| | AGCCAACAGTCTCAAGCTCAAAGCCAGGAACAGGAAT | | |
| | CCCAGTCAGCAGAATCTGAATCCGCTGTTCAGTCAAGC | | |
| | AAATCCTCCAGTGGAAGGAGCAGCCAAGCTCAAAGCC | | |
| | AACAACAACAATCTCAGCTCCAACAGTCTCAAGCTCAG | | |
| | AGCCAAGAACAGGAATCCCAATCTGCTCAATCTGGATC | | |
| | CGATGTTGAAGTAAGCAAATCCT | | |
| | CCAGTGGAAGGAGCAGCCAAGCTCAAAGCCAACAACA | | |
| | ACAATCTCAGCTCCAACAGTCCCAAGCTCAGAGCCAAG | | |
| | AACAGGAATCCCAGTCTGCAGGATCTGAATCCGCCGTT | | |
| | CAGTCAAGCAAATCCTCTCAGTGGAATGAGCAGCCAA | | |
| | GCTCAAAGCCAACAACAACAATCTCAGCTCCAACAGTC | | |
| | TCAAGCTCAGAGCCAAGAACAGGAATCCCAATCTGCTC | | |
| | AATCTGGATCCGATGTTGAAGTAAGCAAATCCTCCAGT | | |
| | GGAAGGAGCAGCCAAGCTCAAAGCCAACAACAACAAT | | |
| | CTCAGCTCCAACAGTCCCAAGCTCAGAGCCAAGAACA | | |
| | GGAATCCCAATCTGCTCAATCTGGATCCGATGTTGAAG | | |
| | TCAAGCAAATCCTCCAGTGGAAGGAGCAGCCAAGCTC | | |
| | AAAGCCAACAACAACAATCTCAGCTCCAACAGTCCCAA | | |
| | GCTCAAAGCCAAGAACAGGAATCCCAGTCTGCAGAAT | | |
| | CTGAATCCGATGTTCAGTCAAGCAAATCCTCCAGGGGA | | |
| | ATGAGCAGCCATGCTCAAAGCCAACAGTCTCAAGCTCA | | |
| | AAGCCAGGAACAGGAATCCCAGTCAGCAGAATCTGAA | | |
| | TCCGCCGTTCAGTCAAGCAAATCCTCTCGTGGAATGAG | | |
| | CAGCCAAGCTCAAAGCCAACAACAACAATCTCAGCTCC | | |
| | AACAGTCTCAAGCTCAGAGCCAAGAACAGGAATCCCA | | |
| | ATCTGCTCAATCTGGATCCGATGTTGAAGTAAGCAAAT | | |
| | CCTCCAGTGGAAGGAGCAGCCAAGCTCAAAGCCAACA | | |
| | ACAACAATCTCAGCTCCAACAGTCCCAAGCTCAAAGCC | | |
| | AAGAACAGGAATCCCAGTCTGCAGAATCTGAATCCGAT | | |
| | GTTCAGTCAAGCAAATCCTCCAGGGGAATGAGCAGCC | | |
| | GTGCTCAAAGCCAACAGTCCCAAGCTCAGAGCCAAGA | | |
| | ACAGGAATCCCAATCTGCTCAATCTGGATCCGATGTTG | | |
| | AAGTAAGCAAATCCTCCAGTGGA | | |
| | AGGAGCAGCCAAGCTCAAAGCCAACAACAACAATCTC | | |
| | AGCTCCAACAGTCCCAAGCTCAGAGCCAAGAACAGGA | | |
| | ATCCCAGTCTGCAGGATCTGAATCCGCCGTTCAGTCAA | | |
| | GCAAATCCTCTCAGTGGAATGAGCAGCCAAGCTCAAA | | |
| | GCCAACAACAACAATCTCAGCTCCAACAGTCCCAAGCT | | |
| | CAGAGCCAAGAACAGGAATCCCAGTCTGCAGGATCTG | | |
| | AATCCGCCGTTCAGTCAAGCAAATCCTCTCGTGGAATG | | |
| | AGCAGCCAAGCTCAAAGCCAACAACAACAAGCTCAACT | | |
| | CCAACAGTCCCAAGCTCAAAGCCAAGAACAGGAATCC | | |
| | CAGTCTGCAGAATCTGAATCCGATGTTCAGTCAAGCAA | | |
| | ATCCTCCAGGGGAATGAGCAGCCGTGCTCAAAGCCAA | | |
| | CAGTCCCAAGCTCAGAGCCAAGAACAGGAATCCCAAT | | |
| | CTGCTCAATCTGGATCCGATGTTGAAGTAAGCAAATCC | | |
| | TCCAGTGGAAGGAGCAGCCAAGCTCAAAGCCAACAAC | | |
| | AACAATCTCAGCTCCAACAGTCCCAAGCTCAAAGCCAA | | |
| | GAACAGGAATCCCAGTCTGCAGAATCTGAATCCGATGT | | |
| | TCAGTCAAGCAAATCCTCCAGGGGAATGAGCAGCCAT | | |
| | GCTCAAAGCCAACAGTCTCAAGCTCAAAGCCAGGAAC | | |
| | AGGAATCCCAGTCAGCAGAATCTGAATCCGCCGTTCAG | | |
| | TCAAGCAAATCCTCTCGTGGAATGAGCAGCCAAGCTCA | | |
| | AAGCCAACAACAACAATCTCAGCTCCAACAGTCTCAAG | | |
| | CTCAGAGCCAAGAACAGGAATCCCAATCTGCTCAATCT | | |
| | GGATCCGATGTTGAAGTAAGCAAATCCTCCAGTGGAA | | |
| | GGAGCAGCCAAGCTCAAAGCCAACAACAACAATCTCA | | |
| | GCTCCAACAGTCCCAAGCTCAAAGCCAAGAACAGGAA | | |
| | TCCCAGTCTGCAGAATCTGAATCCGATGTTCAGTCAAG | | |
| | CAAATCCTCCAGGGGAATGAGCAGC | | |
| | CATGCTCAAAGCCAACAGTCCCAAGCTCAGAGCCAAG | | |
| | AACAGGAATCCCAATCTGCTCAATCTGGATCCGATGTT | | |
| | GAAGTAAGCAAATCCTCCAGTGGAAGGAGCAGCCAAG | | |
| | CTCAAAGCCAACAACAACAATCTCAGCTCCAACAGTCC | | |
| | CAAGCTCAAAGCCAAGAACAGGAATCCCAGTCTGCAG | | |
| | AATCTGAATCCGATGTTCAGTCAAGCAAATCCTCCAGG | | |
| | GGAATGAGCAGCCGTGCTCAAAGCCAACAGTCCCAAG | | |
| | CTCAGAGCCAAGAACAGGAATCCCAATCTGCTCAATCT | | |
| | GGATCCGATGTTGACAGTAAGCAAATCCTCCAGTGGAA | | |
| | GGAGCAGCCAAGCTCAAAGCCAACAACAACAATCTCA | | |
| | GCTCCAACAGTCCCAAGCTCAGAGCCAAGAACAGGAA | | |
| | TCCCAGTCTGCAGGATCTGAATCCGCCGTTCAGTCAAG | | |
| | CAAATCCTCTCGTGGAATGAGCAGCCAAGCTCAAAGCC | | |
| | AACAACAACAATCTCAGCTCCAACAGTCCCAAGCTCAG | | |
| | AGCCAAGAACAGGAATCCCAATCTGCTCAATCTGGATC | | |
| | CGATGTTGAAGTAAGCAAATCCTCCAGTGGAAGGAGC | | |
| | AGCCAAGCTCAAAGCCAACAACAACAATCTCAGCTCCA | | |
| | ACAGTCCCAAGCTCAGAGCCAAGAACAGGAATCCCAG | | |
| | TCTGCAGAATCTGAATCCGCTGTTCAGTCAAGCAAATC | | |
| | CTCCAGTGGAATGAGCAGCCAAGCTCAAAGCCAACAA | | |
| | CAACAATCTCAACTCCAACAGTCCCAAGCTCAAAGCCA | | |
| | GGAACAGGAATCTCAATCCGACCAGGCTGAATCAGAT | | |
| | GTTCAGTCAAGCAAATCCTCCAGGGGAATGAGCAGCC | | |
| | ATGCTCAAAGCCAACAGTCTCAAGCTCAAAGCCAGGAA | | |
| | CAGGAATCCCAGTCAGCAGAATCTGAATCCGCCGTTCA | | |
| | GTCAAGCAAATCCTCTCGTGGAATGAGCAGCCAAGCTC | | |
| | AAAGCCAACAACAACAATCTCAG | | |
| | CTCCAACAGTCTCAAGCTCAGAGCCAAGAACAGGAATC | | |
| | CCAATCTGCTCAATCTGGATCCGATGTTGAAGTAAGCA | | |
| | AATCCTCCAGTGGAAGGAGCAGCCAAGCTCAAAGCCA | | |
| | ACAACAACAATCTCAGCTCCAACAGTCCCAAGCTCAAA | | |
| | GCCAAGAACAGGAATCCCAGTCTGCAGAATCTGAATCC | | |
| | GATGTTCAGTCAAGCAAATCCTCCAGGGGAATGAGCA | | |
| | GCCGTGCTCAAAGCCAACAGTCCCAAGCTCAGAGCCA | | |
| | AGAACAGGAATCCCAATCTGCTCAATCTGGATCCGATG | | |
| | TTGACGTAAGCAAATCCTCCAGTGGAAGGAGCAGCCA | | |
| | AGCTCAAAGCCAACAACAACAATCTCAGCTCCAACAGT | | |
| | CCCAAGCTCAGAGCCAAGAACAGGAATCCCAGTCTGC | | |
| | AGGATCTGAATCCGCCGTTCAGTCAAGCAAATCCTCTC | | |
| | GTGGAATGAGCAGCCAAGCTCAAAGCCAACAACAACA | | |
| | ATCTCAGCTCCAACAGTCCCAAGCTCAGAGCCAAGAAC | | |
| | AGGAATCCCAGTCTGCAGGATCTGAATCCGCTGTTCAG | | |
| | TCAAGCAAATCCTCTCAGTGGAATGAGCAGCCAAGCTC | | |
| | AAAGCCAACAACAACAAGCTCAACTCCAACAGTCCCAA | | |
| | GCTCAAAGCCAAGAACAGGAATCCCAGTCTGCAGAAT | | |
| | CTGAATCCGATGTTCAGTCAAGCAAATCCTCCAGGGGA | | |
| | ATGAGCAGCCGTGCTCAAAGCCAACAGTCCCAAGCTC | | |
| | AGAGCCAAGAACAGGAATCCCAATCTGCTCAATCTGGA | | |
| | TCCGATGTTGAAGTAAGCAAATCCTCCAGTGGAAGGA | | |
| | GCAGCCAAGCTCAAAGCCAACAACAACAATCTCAGCTC | | |
| | CAACAGTCCCAAGCTCAAAGCCAAGAACAGGAATCCC | | |
| | AGTCTGCAGAATCTGAATCCGATGTTCAGTCAAGCAAA | | |
| | TCCTCCAGGGGAATGAGCAGCCATGCTCAAAGCCAAC | | |
| | AGTCTCAAGCTCAAAGCCAGGAA | | |
| | CAGGAATCCCAGTCAGCAGAATCTGAATCCGCCGTTCA | | |
| | GTCAAGCAAATCCTCTCGTGGAATGAGCAGCCAAGCTC | | |
| | AAAGCCAACAACAACAATCTCAGCTCCAACAGTCTCAA | | |
| | GCTCAGAGCCAAGAACAGGAATCCCAATCTGCTCAATC | | |
| | TGGATCCGATGTTGAAGTAAGCAAATCCTCCAGTGGAA | | |
| | GGAGCAGCCAAGCTCAAAGCCAACAACAACAATCTCA | | |
| | GCTCCAACAGTCCCAAGCTCAAAGCCAAGAACAGGAA | | |
| | TCCCAGTCTGCAGAATCTGAATCCGATGTTCAGTCAAG | | |
| | CAAATCCTCCAGGGGAATGAGCAGCCATGCTCAAAGC | | |
| | CAACAGTCCCAAGCTCAGAGCCAAGAACAGGAATCCC | | |
| | AATCTGCTCAATCTGGATCCGATGTTGAAGTAAGCAAA | | |
| | TCCTCCAGTGGAAGGAGCAGCCAAGCTCAAAGCCAAC | | |
| | AACAACAATCTCAGCTCCAACAGTCCCAAGCTCAGAGC | | |
| | CAAGAACAGGAATCCCAGTCTGCAGGATCTGAATCCG | | |
| | CCGTTCAGTCAAGCAAATCCTCTCGTGGAATGAGCAGC | | |
| | CAAGCTCAAAGCCAACAACAACAATCTCAGCTCCAACA | | |
| | GTCCCAAGCTCAGAGCCAAGAACAGGAATCCCAGTCT | | |
| | GCAGGATCTGAATCCGCCGTTCAGTCAAGCAAATCCTC | | |
| | TCAGTGGAATGAGCAGCCAAGCTCAAAGCCAACAACA | | |
| | ACAAGCTCAACTCCAACAGTCCCAAGCTCAGAGCCAAG | | |
| | AACAGGAATCCCAATCTGCTCAATCTGGATCCGATGTT | | |
| | GACGTAAGCAAATCCTCCAGTGGAAGGAGCAGCCAAG | | |
| | CTCAAAGCCAACAACAACAATCTCAGCTCCAACAGTCT | | |
| | CAAGCTCAGAGCCAAGAACAGGAATCTCAATCCGACC | | |
| | AGGCTGAATCCGATGTTCAGTCAAGCAAATCCTCCAGG | | |
| | GGAATGAGCAGCCATGCTCAAAGCCAACAGTCTCAAG | | |
| | CTCAAAGCCAGGAACAGGAATCC | | |
| | CAGTCAGCAGAATCTGAATCCGCCGTTCAGTCAAGCAA | | |
| | ATCCTCTCGTGGAATGAGCAGCCAAGCTCAAAGCCAAC | | |
| | AACAACAATCTCAGCTCCAACAGTCTCAAGCTCAGAGC | | |
| | CAAGAACAGGAATCCCAGTCTGCAGAATCTGGATCCG | | |
| | CTGTTGCAGTCAAGCAAATCCTCCAGTGGAAGGAGCA | | |
| | GCCAAGCTCAAAGCCAACAACAACAATCTCAGCTCCAA | | |
| | CAGTCCCAAGCTCAGAGCCAAGAACAGGAATCCCAAT | | |
| | CTGCTCAATCTGGATCCGATGTTGAAGTAAGCAAATCC | | |
| | TCCAGGGGAATGAGCAGCCATGCTCAAAGCCAACAGT | | |
| | CTCAAGCTCAAAGCCAGGAACAGGAATCCCAGTCTGC | | |
| | AGAATCTGAATCCGATGTTCAGTCAAGCAAATCCTCCA | | |
| | GGGGAATGAGCAGCCATGCTCAAAGCCAACAGTCCCA | | |
| | AGCTCAGAGCCAAGAACAGGAATCCCAATCTGCTCAAT | | |
| | CTGGATCCGATGTTGAAGTAAGCAAATCCTCCAGTGGA | | |
| | AGGAGCAGCCAAGCTCAAAGCCAACAACAACAATCTC | | |
| | AGCTCCAACAGTCCCAAGCTCAGAGCCAAGAACAGGA | | |
| | ATCCCAGTCTGCAGGATCTGAATCCGCCGTTCAGTCAA | | |
| | GCAAATCCTCTCGTGGCATGAGCAGCCAAGCTCAAAG | | |
| | CCAACAACAACAATCTCAACTCCAACAGTCCCAAGCTC | | |
| | AAAGCCAGGAACAGGAATCTCAATCCGACCAGGCTGA | | |
| | ATCAGATGTTCAGTCAAGCAAATCCTCCAGGGGAATGA | | |
| | GCAGCCATGCTCAAAGCCAACAGTCTCAAGCTCAAAGC | | |
| | CAGGAACAGGAATCCCAGTCAGCAGAATCTGAATCCG | | |
| | CCGTTCAGTCAAGCAAATCCTCTCGTGGAATGAGCAGC | | |
| | CAAGCTCAAAGCCAACAACAACAATCTCAGCTCCAACA | | |
| | GTCTCAAGCTCAGAGCCAAGAACAGGAATCCCAATCTG | | |
| | CTCAATCTGGATCCGATGTTGAA | | |
| | GTAAGCAAATCCTCCAGTGGAAGGAGCAGCCAAGCTC | | |
| | AAAGCCAACAACAACAATCTCAGCTCCAACAGTCCCAA | | |
| | GCTCAGAGCCAAGAACAGGAATCCCAATCTGCTCAATC | | |
| | TGAATCCGATGTTCAGTCAAGCAAATCCTCTCAGTGGA | | |
| | ATGAGCAGCCAAGCTCAAAGCCAACAACAACAAGCTC | | |
| | AACTCCAACAGTCCCAAGCTCAAAGCCAAGAACAGGA | | |
| | ATCCCAGTCTGCAGAATCTGAATCCGCTGTTCAGTCAA | | |
| | GCAAATCCTCCAGTGGAATGAGCAGCCAAGCTCAAAG | | |
| | CCAGCAACAACAATCTCAACTCCAACAGTCCCAAGCTC | | |
| | AAAGCCAGGAACAGGAATCCCAGTCAGCAGAATCTGA | | |
| | GTCCGCTGTTCAGTCAAGCAAATCCTCCAGTGGAATGA | | |
| | GCAGCCAAGCTCAAAGCCAACAACAGCAATCCCAACT | | |
| | GCAACAGTCCCAAGCTCAGAGCCAAGAACAGGAATCT | | |
| | CAATCCGACCAGGCTGAATCAGATGTTCAGTCAAGCAA | | |
| | ATCCTCCAGGGGAATGAGCAGCCAAGCTCAAAGCCAA | | |
| | CAGTCTCAAGCTCAAAGCCAGGAACAGGAATCCCAGT | | |
| | CAGCAGAATCTGAATCCGCCGTTCAGTCAAGCAAATCC | | |
| | TCCAGTGGAATGAGCAGCCATGCTCAAAGCCAACAGT | | |
| | CTCAAGCTCAAAGCCAGGAACAGGAATCCCAGTCAGC | | |
| | AGAATCTGAATCCGCCGTTCAGTCAAGCAAATCCTCTC | | |
| | GTGGAATGAGCAGCCAAGCTCAAAGCCAACAACAACA | | |
| | ATCTCAGCTCCAACAGTCTCAAGCTCAGAGCCAAGAAC | | |
| | AGGAATCCCAATCTGCTCAATCTGGATCCGATGTTGAA | | |
| | GTAAGCAAATCCTCCAGTGGAAGGAGCAGCCAAGCTC | | |
| | AAAGCCAACAACAACAATCTCAGCTCCAACAGTCCCAA | | |
| | GCTCAGAGCCAAGAACAGGAATCCCAGTCTGCAGGAT | | |
| | CTGAATCCGCCGTTCAGTCAAGCAAA | | |
| | TCCTCCAGTGGAATGAGCAGCCAAGCTCAAAGCCAAC | | |
| | AACAACAATCTCAACTCCAACAGTCCCAAGCTCAAAGC | | |
| | CAAGAACAGGAATCCCAGTCTGCAGAATCTGAATCCGA | | |
| | TGTTCAGTCAAGCAAATCCTCCAGGGGAATGAGCAGC | | |
| | CATGCTCAAAGCCAACAGTCCCAAGCTCAGAGCCAAG | | |
| | AACAGGAATCCCAATCTGCTCAATCTGGATCCGATGTT | | |
| | GAAGTAAGCAAATCCTCCAGTGGAAGGAGCAGCCAAG | | |
| | CTCAAAGCCAACAACAACAATCTCAGCTCCAACAGTCC | | |
| | CAAGCTCAGAGCCAAGAACAGGAATCCCAGTCTGCAG | | |
| | GATCTGAATCCGCCGTTCAGTCAAGCAAATCCTCTCAG | | |
| | TGGAATGAGCAGCCAAGCTCAAAGCCAACAACAACAA | | |
| | GCTCAACTCCAACAGTCCCAAGCTCAGAGCCAAGAACA | | |
| | GGAATCCCAGTCTGCAGAATCTGAATCCGATGTTCAGT | | |
| | CAAGCAAATCCTCCAGGGGAATGAGCAGCCATGCTCA | | |
| | AAGCCAACAGTCTCAAGCTCAAAGCCAGGAACAGGAA | | |
| | TCCCAGTCAGCAGAATCTGAATCCGCTGTTCAGTCAAG | | |
| | CAAATCCTCCAGTGGAATGAGCAGCCAAGCTCAAAGC | | |
| | CAACAACAACAATCTCAGCTCCAACAGTCTCAAGCTCA | | |
| | GAGCCAAGAACAGGAATCCCAATCTGCTCAATCTGGAT | | |
| | CCGATGTTGAAGTAAGCAAATCCTCCAGTGGAAGGAG | | |
| | CAGCCAAGCTCAAAGCCAACAACAACAATCTCAGCTCC | | |
| | AACAGTCCCAAGCTCAGAGCCAAGAACAGGAATCCCA | | |
| | GTCTGCAGGATCTGAATCCGCCGTTCAGTCAAGCAAAT | | |
| | CCTCCAGTGGAATGAGCAGCCAAGCTCAAAGCCAACA | | |
| | ACAACAATCTCAGCTCCAACAGTCCCAAGCTCAGAGCC | | |
| | AAGAACAGGAATCCCAGTCTGCAGGATCTGAATCCGC | | |
| | CGTTGAGTCAAGCAAATCCTCTCGT | | |
| | GGAATGAGCAGCCATGCTCAAAGCCAACAATCTCAAG | | |
| | CTCAAAGCCAGGAACAGGAATCCCAGTCTGCAGGATC | | |
| | TGAATCCGCCGTTCAGTCAAGCAAATCCTCCAGTGGAA | | |
| | TGAGCAGCCAAGCTCAAAGCCAACAACAACAAGCTCA | | |
| | ACTCCAACAGTCCCAAGCTCAGAGCCAAGAACAGGAA | | |
| | TCCCAGTCTGCAGGATCTGAATCCGCCGTTCAGTCAAG | | |
| | CAAATCCTCCAGTGGAATGAGCAGCCAAGCTCAAAGC | | |
| | CAACAACAACAAGCTCAACTCCAACAGTCCCAAGCTCA | | |
| | GAGCCAAGAACAGGAATCCCAGTCTGCAGAATCTGAA | | |
| | TCCGCCGTTCAGTCAAGCAAATCCTCCAGTGGAATGAG | | |
| | CAGCCAAGCTCAAAGCCAACAACAACAATCTCAGCTCC | | |
| | AACAGTCCCAAGCTCAGAGCCAAGAACAGGAATCCCA | | |
| | GTCTGCAGAATCTGAATCCGCTGTTCAGTCAAGCAAAT | | |
| | CCTCCAGTGGAATGAGCAGCCAAGCTCAAAGCCAACA | | |
| | ACAACAAGCTCAACTCCAACAGTCCCAAGCTCAGAGCC | | |
| | AAGAACAGGAATCCCAGTCTGCAGGATCTGAATCCGC | | |
| | CGTTCAGTCAAGCAAATCCTCCAGTGGAATGAGCAGCC | | |
| | AAGCTCAAAGCCAACAACAACAAGCTCAACTCCAACAG | | |
| | TCCCAAGCTCAGAGCCAAGAACAGGAATCCCAGTCTG | | |
| | CAGGATCTGAATCCGCCGTTCAGTCAAGCAAATCCTCC | | |
| | AGTGGAATGAGCAGCCAAGCTCAAAGCCAACAACAAC | | |
| | AATCTCAGCTCCAACAGTCCCAAGCTCAGAGCCAAGAA | | |
| | CAGGAATCGCAGTCTGCAGAATCTGAATCCGCCGTTG | | |
| | AGTCAAGCAAATCCTCTCAGTGGAATGAGCAGCCATGC | | |
| | TCAAAGCCAACAGTCTCAAGCTCAAAGCCAGGAACAG | | |
| | GAATCCCAGTCTGCAGGATCTGAATCCGCCGTTCAGTC | | |
| | AAGCAAATCCTCCAGTGGAATGAGC | | |
| | AGCCAAGCTCAAAGCCAACAACAACAAGCTCAACTCCA | | |
| | ACAGTCCCAAGCTCAGAGCCAAGAACAGGAATCCCAG | | |
| | TCTGCAGGATCTGAATCCGCCGTTCAGTCAAGCAAATC | | |
| | CTCCAGTGGAATGAGCAGTCAAGCTCAAAGCCAACAG | | |
| | TCTCAAGCTCAAAGCCAGGAACAGGAATCCCAATCTGC | | |
| | TCAATCTGGATCCGACGTTCAAGTAAGCAAATCGTCCA | | |
| | GTGGAATGAGCAGCCAAGCTCAAAGCCAACAACAGCA | | |
| | AGCTCAACTCCAACAGTCCCAAGCTCAGAGCCAAGAAC | | |
| | AGGAATCCCAGTCTGCAGGATCTGAATCCGCCGTTCA | | |
| | GTCAAGCAAATCCTCCAGTGGAATGAGCAGCCAAGCT | | |
| | CAAAGCCAACAACAACAATCTCAGCTCCAACAGTCCCA | | |
| | AGCTCAGAGCCAAGAACAGGAATCCCAGTCTGCAGGA | | |
| | TCTGAATCCGCCGTTGAGTCAAGCAAATCCTCTCAGTG | | |
| | GAATGAGCAGCCATGCTCAAAGCCAACAATCTCAAGCT | | |
| | CAAAGCCAGGAACAGGAATCCCAGTCTGCAGGATCTG | | |
| | AATCCGCCGTTCAGTCAAGCAAATCCTCCAGTGGAATG | | |
| | AGCAGCCAAGCTCAAAGCCAACAACAACAAGCTCAACT | | |
| | CCAACAGTCCCAAGCTCAGAGCCAAGAACAGGAATCC | | |
| | CAGTCTGCAGGATCTGAATCCGCCGTTCAGTCAAGCAA | | |
| | ATCCTCCAGTGGAATGAGCAGCCAAGCTCAAAGCCAA | | |
| | CAACAACAAGCTCAACTCCAACAGTCCCAAGCTCAGAG | | |
| | CCAAGAACAGGAATCCCAGTCTGCAGAATCTGAATCCG | | |
| | CCGTTCAGTCAAGCAAATCCTCCAGTGGAATGAGCAGC | | |
| | CAAGCTCAAAGCCAACAACAACAATCTCAGCTCCAACA | | |
| | GTCCCAAGCTCAGAGCCAAGAACAGGAATCCCAGTCT | | |
| | GCAGGATCTGAATCCGCCGTTCAGTCAAGCAAATCCTC | | |
| | CAGTGGAATGAGCAGCCAAGCT | | |
| | CAAAGCCAACAACAACAAGCTCAACTCCAACAGTCCCA | | |
| | AGCTCAGAGCCAAGAACAGGAATCCCAGTCTGCAGGA | | |
| | TCTGAATCAGCCGTTCAGTCAAGCAAATCCTCCAGTGG | | |
| | AATGAGCAGCCAAGCTCAAAGCCAACAACAACAATCTC | | |
| | AGCTCCAACAGTCCCAAGCTCAGAGCCAAGAACAGGA | | |
| | ATCGCAGTCTGCAGAATCTGAATCCGCCGTTGAGTCAA | | |
| | GCAAATCCTCTCGTGGAATGAGCAGCCATGCTCAAAGC | | |
| | CAACAATCTCAAGCTCAAAGCCAGGAACAGGAATCCCA | | |
| | GTCTGCAGGATCTGAATCCGCCGTTCAGTCAAGCAAAT | | |
| | CCTCCAGTGGAATGAGCAGCCAAGCTCAAAGCCAACA | | |
| | ACAACAAGCTCAACTCCAACAGTCCCAAGCTCAGAGCC | | |
| | AAGAACAGGAATCCCAGTCTGCAGGATCTGAATCAGC | | |
| | CGTTCAGTCAAGCAAATCCTCCAGTGGAATGAGCAGTC | | |
| | AAGCTCAAAGCCAACAGTCTCAAGCTCAAAGCCAGGA | | |
| | ACAGGAATCCCAATCTGCTCAATCTGGATCCGACGTTC | | |
| | AAGTAAGCAAATCGTCCAGTGGAATGAGCAGCCAAGC | | |
| | TCAAAGCCAACAACAGCAAGCTCAAGTCCAACAGTCTC | | |
| | AAGCTCAGAGCCAAGAACAGGAATCCCAATCTGCTCAA | | |
| | TCTGAATCCGCCATTCAGTCAAGCAAATCCTCCAGTGG | | |
| | AATGAGCAGCCAAGCTCAAAGCCAACAACAGCAAGCT | | |
| | CAAGTCCAACAGTCTCAAGCTCAGAGCCAAGAACAGG | | |
| | AATCCCAATCTGCTCAATCTGGATCCGACGTTCAAGTA | | |
| | AGCAAATCCTCCAGTGGTAGGAGCAGCCAAGCTCAAA | | |
| | GCCAACAACAACAATCTCAAGTCCAACAGTCCCAAGCT | | |
| | CAGAGCCAAGAACAGGAATCCCAGTCTGCAGGATCTG | | |
| | AATCAGCCGTTCAGTCAAGCAAATCCTCCAGTGGAATG | | |
| | AGCAGCCAAGCTCAAAGCCAAC | | |
| | AACAACAATCTCAGCTCCAACAGTCCCAAGCTCAGAGC | | |
| | CAAGAACAGGAATCGCAGTCTGCAGAATCTGAATCCG | | |
| | CCGTTGAGTCAAGCAAATCCTCTCGTGGAATGAGCAG | | |
| | CCATGCTCAAAGCCAACAGTCTCAAGCTCAAAGCCAGG | | |
| | AACAGGAATCCCAGTCTGCAGGATCTGAATCCGCCGTT | | |
| | CAGTCAAGCAAATCCTCCAGTGGAATGAGCAGCCAAG | | |
| | CTCAAAGCCAACAACAACAAGCTCAACTCCAACAGTCC | | |
| | CAAGCTCAGAGCCAAGAACAGGAATCCCAGTCTGCAG | | |
| | GATCTGAATCCGCCGTTCAGTCAAGCAAATCCTCCAGT | | |
| | GGAATGAGCAGCCAAGCTCAAAGCCAACAACAACAAG | | |
| | CTCAACTCCAACAGTCCCAAGCTCAGAGCCAAGAACAG | | |
| | GAATCCCAGTCTGCAGGATCTGAATCAGCCGTTCAGTC | | |
| | AAGCAAATCCTCCAGTGGAATGAGCAGTCAAGCTCAAA | | |
| | GCCAACAGTCTCAAGCTCAAAGCCAGGAACAGGAATC | | |
| | CCAATCTGCTCAATCTGGATCCGACGTTCAAGTCAAGC | | |
| | AAATCCTCCAGTGGAATGAGCAGCCAAGCTCAAAGCC | | |
| | AACAACAGCAAGCTCAAGTCCAACAGTCTCAAGCTCAG | | |
| | AGCCAAGAACAGGAATCCCAATCTGCTCAATCTGAATC | | |
| | CGCCATTCAGTCAAGCAAATCCTCCAGTGGAATGAGCA | | |
| | GCCAAGCTCAAAGCCAACAACAGCAAGCTCAAGTCCA | | |
| | ACAGTCTCAAGCTCAGAGCCAAGAACAGGAATCCCAAT | | |
| | CTGCTCAATCTGGATCCGACGTTCAAGTCAAGCAAATC | | |
| | CTCCAGTGGTAGGAGCAGCCAAGCTCAAAGCCAACAA | | |
| | CAACAATCTCAAGTCCAACAGTCCCAAGCTCAGAGCCA | | |
| | AGAACAGGAATCCCAGTCTGCAGGATCTGAATCAGCC | | |
| | GTTCAGTCAAGCAAATCCTCCAGTGGAATGAGCAGCCA | | |
| | AGCTCAAAGCCAACAACAACAA | | |
| | GCTCAACTCCAACAGTCCCAAGCTCAGAGCCAAGAACA | | |
| | GGAATCCCAGTCTGCAGGATCTGAATCAGCCGTTCAGT | | |
| | CAAGCAAATCCTCCAGTGGAATGAGCAGTCAAGCTCAA | | |
| | AGCCAACAGTCTCAAGCTCAAAGCCAGGAACAGGAAT | | |
| | CTCAATCTGCTCAATCTGGATCCGCCGTTCAGTCAAGT | | |
| | AAATCCTCCAGTGGAATGAGCAGCCAAGCTCAAAGCC | | |
| | AACAACAGCAAGCTCAAGTCCAACAGTCTCAAGCTCAG | | |
| | AGCCAAGAACAGGAATCCCAATCTGCTCAATCTGAATC | | |
| | CGCCATTCAGTCAAGCAAATCCTCCAGTGGAATGAGCA | | |
| | GCCAAGCTCAAAGCCAACAACAGCAAGCTCAAGTCCA | | |
| | ACAGTCTCAAGCTCAGAGCCAAGAACAGGAATCCCAAT | | |
| | CTGCTCAATCTGGATCCGACGTTCAAGTAAGCAAATCC | | |
| | TCCAGTGGTAGGAGCAGCCAAGCTCAAAGCCAACAAC | | |
| | AACAATCTCAAGTCCAACAGTCTCAAGCTCAGAGCCAA | | |
| | GAACAGGAATCCCAATCTGCCGAATCTGAATCGGCTGT | | |
| | GCAGTCAAGCAAATCCTCCAGTGGAAGGAGCAGCCAG | | |
| | GCTCAAAGCCAACAACAACAAGCTCAACTCCAACAGTC | | |
| | TCAAGCTCAGAGTCAAGAAGAGGAATCTGAATCTGCTC | | |
| | AGTCTGAATCCGAAGTTCATGTAAGCAAATCGTCAGGT | | |
| | GGAAAGAGCAGCCAAGTGCAGAGTGAAGAACAAGAAT | | |
| | CTGAAGTCGAAGAATCCCAATCTGAAAAACAAGTAGAA | | |
| | GACTCCGAATCTGGTAAGGCCGTATCTGTTAGTCATAG | | |
| | TAGCAAATCTGCAAGTAAAAAGTGCAGCCATTCTTCAC | | |
| | ACAAACACCATGGATCTGAGGGTAAACATTCGAAATCC | | |
| | ACACACAATGTACACTCACATTCAAGCCATGAGAGTAG | | |
| | TCATTCCGGACATAAACATCACGAGTCTGAAGTTGAAT | | |
| | CTGAACAGGAGGAATCGT | | |
| | TGGTCGGCGAACAGATGAAGTGGAAGCAACTGCCAGC | | |
| | GTTGAAGCTTAAGTTGAACCGAAAACGTTTGTCCCTTA | | |
| | ACCGAATCAAGGTGCACTAGGAATTCATGTGATGGGT | | |
| | GAAAAGAATGAATCCTTTTTAGATGGATGACACTACTT | | |
| | TTACACTGACCGGCTTAGATTTAAGGAACACGTTAGAT | | |
| | AATTATTATGAATTTATATAATTCGTAATTTGCCACATTT | | |
| | GCAGTAGATGGTAGTATTAGATAGTAGATGCAACTGTG | | |
| | CAACGCTTTGTAGTGAATTAAAATAGATGTATTTACAG | | |
| | CAATCACTTTTTGTTTTTATTTGGTTGATGCATTGTGAT | | |
| | GATCTGTCCTCCTTATACTGTTTGATATTATACGAGAAG | | |
| | CAACTTTCGCATATCATAATCGAGGTTCTTCTACACATT | | |
| | ATGTTACTATACAGCTACTTTATAATAACGGTTGAAGC | | |
| | GATAAGAGGGAAGACGAAGTTTTCCACGACAACATATC | | |
| | TACCTATGGTGTCGTACAGCAAGCGGTACCACAAAAAG | | |
| | GAAGTGTCAAACTTTTCCTATTCCGTTTATTGTTGGACG | | |
| | CTCTATTACGAATACTGCAATTCTCCTATTAGAAGTTTG | | |
| | AGCTGTATATCGTTACGTATGCGACGATCGTTGTTCTA | | |
| | ACGCGTGAACTTTCTGTGGACATCGGACGCGTTATCGA | | |
| | GCCGACGTTCGCATTTTACATTTTCCCTGTGGCTTTGTT | | |
| | CACTCGGGCGTCACTTCGATCCGATTGTCGCGTCTGAA | | |
| | AAGCGGCAACGATATTTTCTCTATTTCCATCGATGCGC | | |
| | ACCGTGTGTCTGGCCGGATAATTAAACGGAACGGACT | | |
| | GACCTCCTTCCAATCCCCGAGCAACATGG | | |
| 26 | SKSTHTAHKSSGGKSSQME | AA | trypsin digest |
| | | | product |
| 27 | PTTTSSTPTVPSSEPR | AA | trypsin digest |
| | | | product |
| 28 | TGIPICSIWIR | AA | trypsin digest |
| | | | product |
| 29 | SSQWNEQPSSK | AA | trypsin digest |
| | | | product |
| 30 | NLYFQGAK | AA | trypsin digest |
| | | | product |
| 31 | KHHGHPNHHK | AA | trypsin digest |
| | | | product |
| 32 | TLKFHPHH | AA | trypsin digest |
| | | | product |
| 33 | SSSGMSSQAQSQQQQAQLQQSQDQRQEQESQSAGSE | | the longest |
| | SAVKSSKSSSGMSSQAQSQQQQAQLQQSQAQSQEEE | | uninterrupte |
| | SESAQSESEVHVSK | | d amino acid |
| | | | sequence |
| | | | from the |
| | | | mass spec of |
| | | | the nest |
| | | | material |
| 34 | SQEQESQSAGSESAVQSSKSSSGMSSQAQSQQQQSQ | AA | Mass spec |
| | LQQSQAQSQEQESQSAGSESAVESSKSSRGMS | | transcriptome |
| 35 | SQLQQSQAQSQEQESQSAESESAVQSSKSSSGMSSQA | AA | Mass spec |
| | QSQQQQAQLQQSQAQSQEEESESAQSESEVHVSK | | transcriptome |
| 36 | KSSSGMSSQAQSQQQQSQLQQSQAQSQEQESQSAES | AA | Mass spec |
| | ESAVESSK | | transcriptome |
| 37 | GMSSQAQSQQQQSQLQQSQAQSQEQ | AA | Mass spec |
| | | | transcriptome |
| 38 | ESQSAQSGMSSQAQSQQQQAQVQQSQAQRQEQESQS | AA | Mass spec |
| | AGSESAVKSSK | | transcriptome |
| 39 | SSVMISQSQSQQQQAQVQQSQAQSQEQESQSAQSGS | AA | Mass spec |
| | DVQVSK | | transcriptome |
| 40 | QAQVQQSQAQSQEQESQSAQSESAVQTSKSSSGMRS | AA | Mass spec |
| | QSQSQQQQAKLQQSQAQSQEQE | | transcriptome |
| 41 | GMSSHSQSQQSQAQSQEQESQSAGSESAVQSSK | AA | Mass spec |
| | | | transcriptome |
| 42 | SQQQQAQVQQSQAQ | AA | Mass spec |
| | | | transcriptome |
| 43 | IQQSQAQSQEQESQSAGSESAVQSSK | AA | Mass spec |
| | | | transcriptome |
| 44 | SSQAQSQQQQSQVQQSQAQRQEQESQSAGSESAVKS | AA | Mass spec |
| | SKSSSGMSSQAQSQQQQ | | transcriptome |
| 45 | SQQQSQLQQSQAQSQQQESQSDQAESDVQSSK | AA | Mass spec |
| | | | transcriptome |
| 46 | QSAGCESAVQSSHSSSGMSSLAQSQQQQSQLQQSQA | AA | Mass spec |
| | QRK | | transcriptome |
| 47 | MKCVLLLVVIFAVAAYA | AA | FSS |
| | | | ((FUNSecSf9) |
| | | | secretion |
| | | | tag |
| 48 | MKCVLLLVVIFAVAAYA | AA | FSS |
| | | | ((FUNSecSf9) |
| | | | secretion |
| | | | tag |
| 49 | ATGCGCCCTCAGTCTCCACATCACGGCCATCACGGTGC | NA | HnM1M7-03 |
| | TAAAACTACGCTGAAGTTTCACCCGCATCACGGCTTTC | | |
| | GTAAGCATCATGGTCACCCGAACCACCATAAGGCCAG | | |
| | CAACAGCGAGAAAGAACTGGAGTGGGATGATAACGTT | | |
| | CAGCAGAAATATTGGAATGAAAAGCGCCTGCAAAGCG | | |
| | ACCGTAAACACGAGAGCGAATTCGAGCAATCCAAGAG | | |
| | CGAGAATAAAGTCACCAAGAGCAGCAGCACGCACAGC | | |
| | AGCCAACACAACGAAAATCACACTGAGCTGAGCGAGT | | |
| | CGAAGTCCACCCATACCGCGCATAAGTCGAGCGGTGG | | |
| | CAAGAGCAGCCAAATGGAAAGCCAACACCAGCAAGCA | | |
| | CAAAATCAGCAATCCCAAGCGCAAAGCCAAGAACAAG | | |
| | AGAGCCAATCCGCGGAAAGCGAATCGGCGGTGCAGA | | |
| | GCTCACAATCTAGCAGCGGTATGTCCAGCCAAGCTCAA | | |
| | AGCCAACAGCAACAAGCCCAAAGCCAGGAACAAGAGT | | |
| | CCCAGAGCATGAGCAGCCGTGCGCAGTCCCAGCAGTC | | |
| | TCAAGCACAATCTCAGGAGCAGGAGTCTCAAAGCGCA | | |
| | CAGAGCGGCTCTGACGTTGAGGTGTCCAAGTCCAGCT | | |
| | CCGGTCGCTCTAGCCAGGCCCAGTCTCAGCAGCAGCA | | |
| | ATCTCAACTGCAACAATCTCAAGCTCAGAGCCAAGAGC | | |
| | AAGAATCTCAATCTGCAGAAAGCGAGAGCGATGTTCAA | | |
| | TCCAGCAAATCTAGCCGTGGCATGAGCTCGCATGCAC | | |
| | AGAGCCAACAGAGCCAAGCGCAATCTCAAGAACAGGA | | |
| | ATCCCAAAGCGCCGAGAGCGAAAGCGCAGTTCAAAGC | | |
| | TCTAAAAGCTCTCGCGGCATGTCAAGCCAGGCTCAGTC | | |
| | ACAACAGCAGCAAAGCCAGCTGCAACAGTCTCAGGCT | | |
| | CAATCACAAGAGCAAGAATCCCAGTCCGCGCAGTCTG | | |
| | GCAGCGACGTTGAAGTGAGCAAATCGAGCTCTGGTAG | | |
| | ATCTTCACAAGCGCAGAGCCAGCAACAACAGTCCCAAT | | |
| | TGCAACAATCCCAGGCACAGTCGCAAGAGCAGGAAAG | | |
| | CCAGTCCGCAGAGAGCGAGTCTGACGTCCAGAGCAGC | | |
| | AAATCTTCTCGCGGTATGAGCAGCCACGCGCAGTCGC | | |
| | AGCAGTCCCAAGCCCAGTCCCAGGAACAGGAGAGCCA | | |
| | AAGCGCTCAGAGCGGTAGCGATGTCGAGGTGAGCAAA | | |
| | AGCAGCAGCGGCCGTAGCAGCCAGGCACAATCGCAGC | | |
| | AACAGCAGAGCCAACTGCAACAAAGCCAAGCACAAAG | | |
| | CCAGGAACAAGAGTCCCAGTCTGCTGGTTCCGAGAGC | | |
| | GCCGTTCAGAGCTCCAAAAGCAGCCGTGGTATGAGCT | | |
| | CTCAAGCCCAATCCCAACAACAGCAGTCGCAACTGCAG | | |
| | CAATCGCAGGCGCAATCCCAGGAGCAAGAGTCTCAGA | | |
| | GCGCGGGTAGCGAGAGCGCGGTCCAGTCTAGCAAAA | | |
| | GCTCCCAGTGGAATGAACAGCCGAGCAGCAAGCCGAC | | |
| | CACGACCAGCAGCACCCCTACCGTCCCGTCCAGCGAA | | |
| | CCGCGTACGGGCATTCCGATTTGCAGCATCTGGATCC | | |
| | GCTGT | | |
| 50 | MRPQSPHHGHHGAKTTLKFHPHHGFRKHHGHPNHHKA | AA | HnM1M7-03 |
| | SNSEKELEWDDNVQQKYWNEKRLQSDRKHESEFE | | |
| | QSKSENKVTKSSSTHSSQHNENHTELSESKSTHTAHKS | | |
| | SGGKSSQMESQHQQAQNQQSQAQSQEQESQSAE | | |
| | SESAVQSSQSSSGMSSQAQSQQQQAQSQEQESQSMS | | |
| | SRAQSQQSQAQSQEQESQSAQSGSDVEVSKSSSGRSS | | |
| | QAQSQQQQSQLQQSQAQSQEQESQSAESESDVQSSK | | |
| | SSRGMSSHAQSQQSQAQSQEQESQSAESESAVQSSKS | | |
| | SRGMSSQAQSQQQQSQLQQSQAQSQEQESQSAQSGS | | |
| | DVEVSKSSSGRSSQAQSQQQQSQLQQSQAQSQEQES | | |
| | QSAESESDVQSSKSSRGMSSHAQSQQSQAQSQEQES | | |
| | QSAQSGSDVEVSKSSSGRSSQAQSQQQQSQLQQSQA | | |
| | QSQEQESQSAGSESAVQSSKSSRGMSSQAQSQQQQS | | |
| | QLQQSQAQSQEQESQSAGSESAVQSSKSSQWNEQPSS | | |
| | KPTTTSSTPTVPSSEPRTGIPICSIWIRC | | |
| 51 | ATGAGCAGCCGTGCGCAATCCCAGCAATCCCAAGCAC | NA | HnM7-06 |
| | AGTCCCAAGAACAGGAATCCCAGAGCGCGCAATCTGG | | |
| | TAGCGATGTCGAAGTGAGCAAATCCTCTAGCGGTCGC | | |
| | AGCAGCCAAGCCCAGTCTCAACAGCAGCAGTCTCAACT | | |
| | GCAGCAGTCGCAGGCGCAGTCACAAGAGCAAGAATCC | | |
| | CAGTCGGCGGAGAGCGAGAGCGATGTGCAGTCCAGC | | |
| | AAGAGCAGCCGCGGCATGTCTAGCCACGCGCAAAGCC | | |
| | AGCAATCTCAGGCACAAAGCCAGGAGCAAGAAAGCCA | | |
| | GTCTGCAGAAAGCGAGAGCGCTGTCCAGAGCTCTAAG | | |
| | TCCAGCCGTGGCATGTCCTCTCAGGCCCAGTCCCAACA | | |
| | ACAACAAAGCCAGTTGCAGCAAAGCCAAGCGCAGAGC | | |
| | CAAGAACAAGAATCGCAGAGCGCTCAGAGCGGCAGCG | | |
| | ATGTGGAAGTTTCTAAGAGCAGCAGCGGCCGTTCTAG | | |
| | CCAGGCACAGAGCCAGCAGCAACAATCTCAGCTGCAG | | |
| | CAATCGCAAGCCCAAAGCCAAGAGCAGGAATCTCAGA | | |
| | GCGCAGAGAGCGAAAGCGACGTGCAGAGCAGCAAGA | | |
| | GCTCACGTGGCATGAGCAGCCACGCCCAATCGCAGCA | | |
| | GAGCCAGGCTCAGTCGCAAGAGCAAGAGTCGCAAAGC | | |
| | GCGCAGAGCGGTAGCGACGTCGAAGTTTCCAAGTCCA | | |
| | GCAGCGGTCGTAGCAGCCAGGCCCAGAGCCAACAACA | | |
| | GCAGTCCCAACTGCAACAGTCCCAGGCGCAATCGCAG | | |
| | GAACAGGAAAGCCAGAGCGCAGGCAGCGAAAGCGCT | | |
| | GTTCAAAGCTCCAAATCTAGCCGTGGTATGTCCAGCCA | | |
| | AGCTCAGTCACAGCAGCAGCAATCACAGCTGCAACAAT | | |
| | CCCAGGCACAATCTCAGGAACAAGAGAGCCAAAGCGC | | |
| | GGGTTCCGAGAGCGCGGTTCAATCTAGCAAAAGCTCG | | |
| | CAGTGGAACGAACAGCCGTCCAGCAAACCGACCACTA | | |
| | CCAGCAGCACCCCAACGGTTCCGAGCAGCGAGCCGAG | | |
| | AACGGGTATTCCGATCTGTAGCATTTGGATCCGCTGC | | |
| 52 | MSSRAQSQQSQAQSQEQESQSAQSGSDVEVSKSSSG | AA | HnM7-06 |
| | RSSQAQSQQQQSQLQQSQAQSQEQESQSAESESDVQ | | |
| | SSKSSRGMSSHAQSQQSQAQSQEQESQSAESESAVQS | | |
| | SKSSRGMSSQAQSQQQQSQLQQSQAQSQEQESQSAQ | | |
| | SGSDVEVSKSSSGRSSQAQSQQQQSQLQQSQAQSQE | | |
| | QESQSAESESDVQSSKSSRGMSSHAQSQQSQAQSQE | | |
| | QESQSAQSGSDVEVSKSSSGRSSQAQSQQQQSQLQQ | | |
| | SQAQSQEQESQSAGSESAVQSSKSSRGMSSQAQSQQ | | |
| | QQSQLQQSQAQSQEQESQSAGSESAVQSSKSSQWNE | | |
| | QPSSKPTTTSSTPTVPSSEPRTGIPICSIWIRC | | |

### INDUSTRIAL APPLICATION

The invention has industrial application in the production of a polypeptide that is useful to provide an amphipathic, hydrophobic, hygroscopic and/or hydrophilic coating to a wide variety of articles of manufacture, including but not limited to synthetic fibers, textiles, and components thereof and biomedical devices and components thereof.

1. An isolated polynucleotide encoding an isolated polypeptide comprising at least 70% amino acid sequence identity to SEQ ID NO: 50, 2, 4, 6, 8, 10, 12, 14, 16, 18, 20, 22, or 52.

2. The isolated polynucleotide of claim 1 comprising at least 70% nucleic acid sequence identity to SEQ ID NO: 49, 1, 3, 5, 7, 9, 11, 13, 15, 17, 19, 21, or 51.

3. (canceled)

4. A proteinaceous polymer comprising at least a portion of a polypeptide as defined in claim 1.

5. A vector comprising an isolated polynucleotide of claim 1.

6. (canceled)

7. An isolated host cell comprising an isolated polynucleotide as defined in claim 1.

8. (canceled)

9. (canceled)

10. (canceled)

11. A composition comprising an isolated polynucleotide as defined in claim 1 a proteinaceous polymer of claim 4, a vector of claim 5 or an isolated host cell of claim 7 and a carrier, diluent or excipient.

12. (canceled)

13. (canceled)

14. A method of coating an article of manufacture or component or part thereof with an isolated polypeptide comprising at least 70% amino acid sequence identity to SEQ ID NO: 50, 2, 4, 6, 8, 10, 12, 14, 16, 18, 20, 22, and 52.

15. The method of claim 14 wherein the coating is a film.

16. The method of claim 14 wherein the article of manufacture is selected from the group consisting of textiles or components or parts thereof and biomedical devices or components or parts thereof.

17. The method of claim 14 wherein the article of manufacture or component or part thereof is a synthetic fiber.

18. (canceled)

19. The method of claim 17 wherein the synthetic fiber is selected from the group consisting of polyester, spandex, rayon, nylon, acrylic, microfiber, neoprene, polyamide, acetate, polyvinyl chloride (PVC) and synthetic or “faux” leather and fur fibers.

20. The method of claim 14 wherein the article of manufacture or component or part thereof is a natural fiber.

21. The method claim 20 wherein the natural fiber is selected from the group consisting of cotton, wool, silk, coir, alpaca, flax, hemp, bamboo, sisal and jute.

22. The method of claim 14 wherein the article of manufacture is a natural textile or a synthetic textile.

23. The method of claim 14 wherein the article of manufacture or component or part thereof is or is comprised in or on a biomedical device.

24. The method of claim 23 wherein the biomedical device is an implantable biomedical device.

25. The method of claim 24 wherein the implantable biomedical device is selected from the group consisting of cardiovascular devices including cardioverter defibrillators, pacemakers and left ventricular assist devices, breast implants, cochlear implants, intraocular lenses, joint replacements including hip implants, catheters, dialysis tubing, contraceptive intrauterine devices, stents, sutures, staples, bandages, and wound dressings.

26. The method of claim 14 wherein the article of manufacture is an air filtration device or component or part thereof.

27. (canceled)

28. (canceled)

29. (canceled)


SEQUENCE LISTING (52 sequences)

SEQ ID NO: 1 - DNA, 16353 bp, Hylaeus nubilosus
  source 1..16353: mol_type = other DNA
atgaaatgtgtcttgttattagttgtcatattcgcagtcgcggcttatgcccgacctcagtcgccacaccatggtcaccatggtgcgaagactactttgaaattccatccccaccatggattccgcaaacatcacggccatcctaatcatcataaagcttccaacagcgaaaaagaactagaatgggatgacaacgtacagcagaaatactggaatgagaaacgactccaatccgatagaaaacatgaatcagaattcgaacaatccaaatccgaaaacaaagtaacaaaatcgtccagtacacactctagccaacataatgagaaccatacggaactatctgagtcaaaatccacacatacggcacataaatcgtcaggaggaaaatcaagccaaatggaaagccaacaccaacaagctcaaaaccaacagtcccaagcccagagccaagaacaggaatcccagtctgcagaatctgaatccgctgttcagtcaagccaatcctccagtggaatgagcagccaagctcaaagccaacaacaacaagcccagagccaagaacaggaatcccagtcctgcagaatcagaatccgctgttcagtcaagccaatcctccagtggaatgagcagccaagctcaaagccaacaacaacaagcccagagccaagaacaggaatcccagtctgcagaatcatgagcagccaagctcaaagccaacaacagcaagctcaagtccaacagtctcaagctcagagccaagaacaggagtcccagtctgcaggatctgaatccgccgttcagtcaagcaaatcctccagtggaatgagcagccaagctcaaagccaacagtcccaagctcagagccaagaacaggaatcccaatctgctcaatctggatccgatgttcaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggagtcacagtctgcaggatctgaatccgccgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagtagccaagctcaaagccaacaacaacaatctcagctccaacagtcccaagctcagagccaagaacaggaatcccagtctgcagaatctgaatccgccgttcagtcaagcaaatcctccagtggaatgagcagccaagctcaaagccaacaacaacaagctcagagccaagaacaggaatcgcaatctgctcaatctgaatccgccattcagtcaagcaaatcctccagtggaatgagcagccaagctcaaagccaacaacagcaagctcaagtccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggagtcccagtctgcaggatctgaatccgccgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagcagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggagtcccagtctgcaggatctgaatccgccgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagtagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaagctcagagccaagaacaggaatcgcaatctgctcaatctgaatccgccattcagtcaagcaaatcctccagtggaatgagcagccaagctcaaagccaacaacagcaagctcaagtccaacagtcccaagctcaaagccaagaacaggaatcccagtctgcagaatctgaatccgatgttcagtcaagcaaatcctccaggggaatgagcagccatgctcaaagccaccagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaagctcaagtccaacagtctcaagctcagagccaagaacaggagtctcaagcaaatcctccagtggaaatgagcagccaagctcaaagccaacaacagcaatctcaactgcaacagtcccaagctcagagccaagaacaggaatctcaatccgaccaggctgaatccgatgttcagtcaagcaaatcctccaggggaatgagcagccacgctcaaagccaacagtctcaagctcaaagccaggaacaggaatcccagtcagcagaatctgagtccgctgttcagtcaagcaaatcctccagtggaaggagcagccaagctcaaagccaacaacaacaatctcagctccaacagtcccaagctcagagccaagaacaggaatcccaatctgctcaatctgcatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtctagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaatctcaactccaacagtcccaagctcagagccaagaacaggaatctcaatccgaccaggctgaatccgatgttcagtcaagcaaatcctccaggggaatgagcagccacgctcaaagccaacagtctcaagctcaaagccaggaacaggaatcccagtcagcagaatctgaatccgctgttcagtcaagcaaatcctccagtggaaggagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaatctcagctccaacagtctcaagctcagagccaagaacaggaatcccaatctgctcaatctggatccgatgtatgttgaagtcaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaatctcagctccaacagtcccaagctcagagccaagaacaggaatcccagtctgcaggatcatgagcagccaagctcaaagccaacaacaacaagctcaactccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgacagtaagcaaatcctccagtggaaggagcagccaagctcaaagccaacaacaacaatctcagctccaacagtcccaagctcagagccaagaacaggaatcccagtctgcaggatcatgagcagccaagctcaaagccaacaacaacaatctcagctccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaatctcaactccaacagtcccaagctcaaagccaggaacaggaatctcaatccgaccaggctgaatcagatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgacgtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaatctcagctccaacagtcccaagctcagagccaagaacaggaatcccagtctgcaggatctgaatccgctgttcagtcaagcaaatcctctcagtggaatgagcagccaagctcaaagccaacaacaacaagctcaactccaacagtcccaagctcaaagccaagaacaggaatcccagtctgcagaatcatgagcagccgtgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaagctcaactccaacagtcccaagctcagagccaagaacaggaatcccaatctgctcaatctggatccgatgtatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccagtctgcagaatctggatccgctgttgcagtcaagcaaatcctccagtggaaggagcagccaagctcaaagccaacaacaacaatctcagctccaacagtcccaagctcagagccaagaacaggaatcccaatctgctcaatctggatccgatgtatgagcagccatgctcaaagccaacagtctcaagctcaaagccaggaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggcatgagcagccaagctcaaagccaacaacaacaatctcaactccaacagtcccaagctcaaagccaggaacaggaatctcaatccgaccaggctgaatcagatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccaatctgctcaatctgaatccgatgttcagtcaagcaaatcctctcagtggaatgagcagccaagctcaaagccaacaacaacaagctcaactccaacagtcccaagctcaaagccaagaacaggaatcccagtctgcagaatcatgagcagccaagctcaaagccagcaacaacaatctcaactccaacagtcccaagctcaaagccaggaacaggaatcccagtcagcagaatctgagtccgctgttcagtcaagcaaatcctccagtggaatgagcagccaagctcaaagccaacaacagcaatcccaactgcaacagtcccaagctcagagccaagaacaggaatctcaatccgaccaggctgaatcagatgttcagtcaagcaaatcctccaggggaatgagcagccaagctcaaagccaacagtctcaagctcaaagccaggaacaggaatcccagtcagcagaatctgaatccgccgttcagtcaagcaaatcctccagtggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaatctcaactccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaagctcaactccaacagtcccaagctcagagccaagaacaggaatcccagtctgcagaatcatgagcagccatgctcaaagccaacagtctcaagctcaaagccaggaacaggaatcccagtcagcagaatctgaatccgctgttcagtcaagcaaatcctccagtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaatctcagctccaacagtcccaagctcagagccaagaacaggaatcccagtctgcaggatctgaatccgccgttgagtcaagcaaatcctctcgtggaatgagcagccatgctcaaagccaacaatctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcagaatctgaatccgccgttcagtcaagcaaatcctccagtggaatgagcagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaatctcagctccaacagtcccaagctcagagccaagaacaggaatcgcagtctgcagaatctgaatccgccgttgagtcaagcaaatcctctggaatgagcagccatgctcaaagccaacaatctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcagaatctgaatccgccgttcagtcaagcaaatcctccagtggaatgagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatcagccgttcagtcaagcaaatcctccagtggaatgagcagccaagctcaaagccaacaacaacaatctcagctccaacagtcccaagctcagagccaagaacaggaatcgcagtctgcagaatctgaatccgccgttgagtcaagcaaatcctctcgtggaatgagcagccatgctcaaagccaacaatctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatcagccgttcagtcaagcaaatcctccagtggaatgagcagtcaagctcaaagccaacagtctcaagctcaaagccaggaacaggaatcccaatctgctcaatctggatccgacgttcaagtaagcaaatcgtccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggaatcccaatctgctcaatctggatccgacgttcaagtaagcaaatcctccagtggtaggagcagccaagctcaaagccaacaacaacaatctcaagtccaacagtcccaagctcagagccaagaacaggaatcccagtctgcaggatctgaatcagccgttcagtcaagcaaatcctccagtggaatgagcagccaagctcaaagccaacaacaacaatctcagctccaacagtcccaagctcagagccaagaacaggaatcgcagtctgcagaatctgaatccgccgttgagtcaagcaaatcctctcgtggaatgagcagccatgctcaaagccaacagtctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatcagccgttcagtcaagcaaatcctccagtggaatgagcagtcaagctcaaagccaacagtctcaagctcaaagccaggaacaggaatcccaatctgctcaatctggatccgacgttcaagtcaagcaaatcctccagtggaatgagcagccaagctcaaagccaacaacagcaagctcaagtccaacagtctcaagctcagagccaagaacaggaatcccaatctgctcaatc

SEQ ID NO: 2 - protein, 5451 aa, Hylaeus nubilosus
MKCVLLLVVIFAVAAYARPQSPHHGHHGAKTTLKFHPHHGFRKHHGHPNHHKASNSEKELEWDDNVQQKYWNEKRLQSDRKHESEFEQSKSENKVTKSSSTHSSQHNENHTELSESKSTHTAHKSSGGKSSQMESQHQQAQNQQSQAQSQEQESQSAESESAVQSSQSSSGMSSQAQSQQQQAQSQEQESQSCRIRIRCSVKPILQWNEQPSSKPTTTSPEPRTGIPVCRIMSSQAQSQQQQAQVQQSQAQSQEQESQSAGSESAVQSSKSSSGMSSQAQSQQSQAQSQEQESQSAQSGSDVQVSKSSSGRSSQAQSQQQQSQLQQSQAQSQEQESQSAGSESAVQSSKSSSGMSSQAQSQQQQAQVQQSQAQSQEQESQSAQSESAIQSSKSSSGMSSQAQSQQQQAQVQQSQAQSQEQESQSAQSESAIQSSKSSSGMSSQAQSQQQQAQVQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSSGMSSQAQSQQQQAQVQQSQAQSQEQESQSAGSESAVQSSKSSSGMSSQAQSQQQQAQVQQSQAQSQEQESQSAGSESAVQSSKSSSGMSSQAQSQQQQAQSQEQESQSAQSESAIQSSKSSSGMSSQAQSQQQQSQLQQSQAQSQEQESQSAESESAVQSSKSSSGMSSQAQSQQQQAQSQEQESQSAQSESAIQSSKSSSGMSSQAQSQQQQAQVQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSSGMSSQAQSQQQQAQVQQSQAQSQEQESQSAGSESAVQSSKSSSGMSSQAQSQQQQAQSQEQESQSAQSESAIQSSKSSSGMSSQAQSQQQQSQLQQSQAQSQEQESQSAESESAVQSSKSSSGMSSQAQSQQQQAQSQEQESQSAQSESAIQSSKSSSGMSSQAQSQQQQAQVQQSQAQSQEQESQSAGSESAVQSSKSSSGMSSQAQSQQQQAQSQEQESQSAQSESAIQSSKSSSGMSSQAQSQQQQSQLQQSQAQSQEQESQSAESESAVQSSKSSSGMSSQAQSQQQQAQSQEQESQSAQSESAIQSSKSSSGMSSQAQSQQQQAQVQQSQAQSQEQESQSAESESDVQSSKSSRGMSSHAQSHQSQAQSQEQESQSAQSGSDVEVSKSSSGRSSQAQSQQQQSQLQQSQAQSQEQESQSAGSESAVQSSKSSSGMSSQAQSQQQQAQVQQSQAQSQEQESQANPPVEMSSQAQSQQQQSQLQQSQAQSQEQESQSDQAESDVQSSKSSRGMSSHAQSQQSQAQSQEQESQSAESESAVQSSKSSSGRSSQAQSQQQQSQLQQSQAQSQEQESQSAQSASDVEVSKSSSGRSSQAQSQQQQSQLQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAGSESAVQSSKSSSGMSSQAQSQQQQSQLQQSQAQSQEQESQSDQAESDVQSSKSSRGMSSHAQSQQSQAQSQEQESQSAESESAVQSSKSSSGRSSQAQSQQQQSQLQQSQAQSQEQESQSAQSGSDVEVSKSSSGRSSQAQSQQQQSQLQQSQAQSQEQESQSAGSESAVQSSKSSQWNEQPSSKPTTTISAPTVSSSEPRTGIPICSIWIRCMLKSSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSRAQSQQSQAQSQEQESQSAQSGSDVEVSKSSSGRSSQAQSQQQQSQLQQSQAQSQEQESQSAGSESAVQSSKSSQWNEQPSSKPTTTISAPTVPSSEPRTGIPVCRIMSSQAQSQQQQAQLQQSQAQSQEQESQSAESESDVQSSKSSRGMSSRAQSQQSQAQSQEQESQSAQSGSDVEVSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSHAQSQQSQAQSQEQESQSAQSGSDVEVSKSSSGRSSQAQSQQQQSQLQQSQAQSQEQESQSAESESDVQSSKSSRGMSSRAQSQQSQAQSQEQESQSAQSGSDVDSKQILQWKEQPSSKPTTTISAPTVPSSEPRTGIPVCRIMSSQAQSQQQQSQLQQSQAQSQEQESQSAQSGSDVEVSKSSSGRSSQAQSQQQQSQLQQSQAQSQEQESQSAESESAVQSSKSSSGMSSQAQSQQQQSQLQQSQAQSQEQESQSDQAESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSRAQSQQSQAQSQEQESQSAQSGSDVDVSKSSSGRSSQAQSQQQQSQLQQSQAQSQEQESQSAGSESAVQSSKSSRGMSSQAQSQQQQSQLQQSQAQSQEQESQSAGSESAVQSSKSSQWNEQPSSKPTTTSSTPTVPSSKPRTGIPVCRIMSSRAQSQQSQAQSQEQESQSAQSGSDVEVSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSRGMSSQAQSQQQQSQLQQSQAQSQEQESQSAGSESAVQSSKSSQWNEQPSSKPTTTSSTPTVPSSEPRTGIPICSIWIRCMSSHAQSQQSQAQSQEQESQSAESESAVQSSKSSRGMSSQAQSQQQQSQLQQSQAQSQEQESQSAESGSAVAVKQILQWKEQPSSKPTTTISAPTVPSSEPRTGIPICSIWIRCMSSHAQSQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSRGMSSQAQSQQQQSQLQQSQAQSQEQESQSDQAESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAQSESDVQSSKSSQWNEQPSSKPTTTSSTPTVPSSKPRTGIPVCRIMSSQAQSQQQQSQLQQSQAQSQEQESQSAESESAVQSSKSSSGMSSQAQSQQQQSQLQQSQAQSQEQESQSDQAESDVQSSKSSRGMSSQAQSQQSQAQSQEQESQSAESESAVQSSKSSSGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAGSESAVQSSKSSSGMSSQAQSQQQQSQLQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSQWNEQPSSKPTTTSSTPTVPSSEPRTGIPVCRIMSSHAQSQQSQAQSQEQESQSAESESAVQSSKSSSGMSSQAQSQQQQSQLQQSQAQSQEQESQSAQSGSDVEVSKSSSGRSSQAQSQQQQSQLQQSQAQSQEQESQSAGSESAVQSSKSSSGMSSQAQSQQQQSQLQQSQAQSQEQESQSAGSESAVESSKSSRGMSSHAQSQQSQAQSQEQESQSAGSESAVQSSKSSSGMSSQAQSQQQQAQLQQSQAQSQEQESQSAGSESAVQSSKSSSGMSSQAQSQQQQAQLQQSQAQSQEQESQSAESESAVQSSKSSSGMSSQAQSQQQQSQLQQSQAQSQEQESQSAESESAVQSSKSSSGMSSQAQSQQQQAQLQQSQAQSQEQESQSAGSESAVQSSKSSSGMSSQAQSQQQQAQLQQSQAQSQEQESQSAGSESAVQSSKSSSGMSSQAQSQQQQSQLQQSQAQSQEQESQSAESESAVESSKSSGMSSHAQSQQSQAQSQEQESQSAGSESAVQSSKSSSGMSSQAQSQQQQAQLQQSQAQSQEQESQSAGSESAVQSSKSSSGMSSQAQSQQQQAQLQQSQAQSQEQESQSAESESAVQSSKSSSGMSSQAQSQQQQSQLQQSQAQSQEQESQSAGSESAVQSSKSSSGMSSQAQSQQQQAQLQQSQAQSQEQESQSAGSESAVQSSKSSSGMSSQAQSQQQQSQLQQSQAQSQEQESQSAESESAVESSKSSRGMSSHAQSQQSQAQSQEQESQSAGSESAVQSSKSSSGMSSQAQSQQQQAQLQQSQAQSQEQESQSAGSESAVQSSKSSSGMSSQAQSQQSQAQSQEQESQSAQSGSDVQVSKSSSGMSSQAQSQQQQAQVQQSQAQSQEQESQSAQSESAIQSSKSSSGMSSQAQSQQQQAQVQQSQAQSQEQESQSAQSGSDVQVSKSSSGRSSQAQSQQQQSQVQQSQAQSQEQESQSAGSESAVQSSKSSSGMSSQAQSQQQQSQLQQSQAQSQEQESQSAESESAVESSKSSRGMSSHAQSQQSQAQSQEQESQSAGSESAVQSSKSSSGMSSQAQSQQQQAQLQQSQAQSQEQESQSAGSESAVQSSKSSSGMSSQAQSQQQQAQLQQSQAQSQEQESQSAGSESAVQSSKSSSGMSSQAQSQQSQAQSQEQESQSAQSGSDVQVKQILQWNEQPSSKPTTASSSPTVSSSEPRTGIPICSI

SEQ ID NO: 3 - DNA, 129 bp, Hylaeus nubilosus
  source 1..129: mol_type = other DNA
atgagcagccaggcccagagccagcagcagcagagccagctgcagcagagccaggcccagagccaggagcaggagagccagagcgcccagagcggcagcgacgtggaggtgagcaagagcagcagcggc

SEQ ID NO: 4 - protein, 43 aa, Hylaeus nubilosus
MSSQAQSQQQQSQLQQSQAQSQEQESQSAXSGSDVXXSKSSSG

SEQ ID NO: 5 - DNA, 129 bp, Hylaeus nubilosus
  source 1..129: mol_type = other DNA
agcgagagcgccgtgcagagcagcaagagcagcagcggcatgagcagccaggcccagagccagcagcagcagagccagctgcagcagagccaggcccagagccaggagcaggagagccagagcgcccag

SEQ ID NO: 6 - protein, 43 aa, Hylaeus nubilosus
  VARIANT 25: note = Any amino acid
  VARIANT 43: note = Any amino acid
SESAVQSSKSSSGMSSQAQSQQQQXQLQQSQAQSQEQESQSAX

SEQ ID NO: 7 - DNA, 114 bp, Hylaeus nubilosus
  source 1..114: mol_type = other DNA
agccagtggaacgagcagcccagcagcaagcccaccaccaccagcagcacccccaccgtgcccagcagcgagcccaggaccggcatccccatctgcagcatctggatcaggtgc

SEQ ID NO: 8 - protein, 38 aa, Hylaeus nubilosus
SQWNEQPSSKPTTTSSTPTVPSSEPRTGIPICSIWIRC

SEQ ID NO: 9 - DNA, 1704 bp, Hylaeus nubilosus
  source 1..1704: mol_type = other DNA
ggatccatgaagtgtgtcctcctgctcgttgttattttcgctgttgctgcttacgcccgcccccagtcgcaccatcaccaccatcacgattacgacatccccaccactgagaacctgtacttccagggcgctaagaccactctgaagttccacccacaccacggattcaggaagcaccacggtcaccctaaccaccacaaggccagcaactctgagaaggaactggagtgggacgacaacgtgcagcagaagtactggaacgaaaagcgcctgcagtctgaccgtaagcacgaatcagagttcgaacagtcaaagtccgagaacaaggtcactaagtccagctctacccactcatcccagcacaacgaaaaccacactgagctgagcgaatctaagtcaactcacaccgctcacaagtcctccggtggcaagtcctcccagatggagtcccagcaccagcaggctcagaaccagcagtcccaggcccagagccaggagcaggaatcccagagcgctgagtctgaatcagccgtgcagtcctcccagtcatccagcggaatgtcttcacaggctcagtctcaacagcagcaggctcaatctcaagaacaggaatcccagagcatgtccagcagggctcagtctcagcagtcacaggcccagtctcaggagcaggaatctcagtcagctcagtccggcagcgacgtggaggtgtccaagtcttcatccggaagaagctctcaggcccagtcccagcagcagcagagccagctgcagcagtctcaggctcagtcacaagaacaggaatctcagtcagccgagtccgaaagcgacgtgcagtcatccaagtcctcccgcggtatgtcatcccacgctcagtctcaacagagccaggctcagtcccaagaacaagaatctcagtcagctgagtccgaaagcgccgtccagtcctccaagtcatcccgtggcatgtcctcccaggctcagtctcagcagcagcagtcacagctgcagcagtcccaggctcaatctcaagagcaagaatctcagtcagcccagtccggtagcgatgtcgaggtgtccaagtcctcctccggtcgctcctcccaagctcaatctcaacaacaacaatctcaattgcaacaatcacaggctcagtcacaagagcaagagtcacaatctgctgagtccgaaagcgacgtccagtccagcaagtcttcaaggggaatgtccagccacgctcaatcacaacaatcacaggcccagtcccaagaacaggagtcacaatcagctcagtccggaagcgacgtggaggtcagcaagtcttcatccggtagaagctctcaagcccaatcacaacaacaacaatctcagttgcaacaatctcaagctcaaagccaagaacaggagtctcagtcagctggctccgaaagcgctgtccagtcatccaagtcctcccgcggtatgtcatcccaggctcaatctcagcaacaacaatctcaactccagcagtctcaggcccagtcacaagagcaagaatcacagtcagctggttccgagtctgctgtgcagtcctccaagtcatcccagtggaacgaacagcctagctctaagcccaccactacctcatccactcctaccgttccgagttctgagccccgcacaggtattccgatttgtagtatttggattcgttgctaaggatcc

SEQ ID NO: 10 - protein, 38 aa, Hylaeus nubilosus; identical to SEQ ID NO: 8

SEQ ID NO: 11 - DNA, 1647 bp, Hylaeus nubilosus
  source 1..1647: mol_type = other DNA
cgcccccagtcgcaccatcaccaccatcacgattacgacatccccaccactgagaacctgtacttccagggcgctaagaccactctgaagttccacccacaccacggattcaggaagcaccacggtcaccctaaccaccacaaggccagcaactctgagaaggaactggagtgggacgacaacgtgcagcagaagtactggaacgaaaagcgcctgcagtctgaccgtaagcacgaatcagagttcgaacagtcaaagtccgagaacaaggtcactaagtccagctctacccactcatcccagcacaacgaaaaccacactgagctgagcgaatctaagtcaactcacaccgctcacaagtcctccggtggcaagtcctcccagatggagtcccagcaccagcaggctcagaaccagcagtcccaggcccagagccaggagcaggaatcccagagcgctgagtctgaatcagccgtgcagtcctcccagtcatccagcggaatgtcttcacaggctcagtctcaacagcagcaggctcaatctcaagaacaggaatcccagagcatgtccagcagggctcagtctcagcagtcacaggcccagtctcaggagcaggaatctcagtcagctcagtccggcagcgacgtggaggtgtccaagtcttcatccggaagaagctctcaggcccagtcccagcagcagcagagccagctgcagcagtctcaggctcagtcacaagaacaggaatctcagtcagccgagtccgaaagcgacgtgcagtcatccaagtcctcccgcggtatgtcatcccacgctcagtctcaacagagccaggctcagtcccaagaacaagaatctcagtcagctgagtccgaaagcgccgtccagtcctccaagtcatcccgtggcatgtcctcccaggctcagtctcagcagcagcagtcacagctgcagcagtcccaggctcaatctcaagagcaagaatctcagtcagcccagtccggtagcgatgtcgaggtgtccaagtcctcctccggtcgctcctcccaagctcaatctcaacaacaacaatctcaattgcaacaatcacaggctcagtcacaagagcaagagtcacaatctgctgagtccgaaagcgacgtccagtccagcaagtcttcaaggggaatgtccagccacgctcaatcacaacaatcacaggcccagtcccaagaacaggagtcacaatcagctcagtccggaagcgacgtggaggtcagcaagtcttcatccggtagaagctctcaagcccaatcacaacaacaacaatctcagttgcaacaatctcaagctcaaagccaagaacaggagtctcagtcagctggctccgaaagcgctgtccagtcatccaagtcctcccgcggtatgtcatcccaggctcaatctcagcaacaacaatctcaactccagcagtctcaggcccagtcacaagagcaagaatcacagtcagctggttccgagtctgctgtgcagtcctccaagtcatcccagtggaacgaacagcctagctctaagcccaccactacctcatccactcctaccgttccgagttctgagccccgcacaggtattccgatttgtagtatttggattcgttgctaaggatcc

SEQ ID NO: 12 - protein, 546 aa, Hylaeus nubilosus
RPQSHHHHHHDYDIPTTENLYFQGAKTTLKFHPHHGFRKHHGHPNHHKASNSEKELEWDDNVQQKYWNEKRLQSDRKHESEFEQSKSENKVTKSSSTHSSQHNENHTELSESKSTHTAHKSSGGKSSQMESQHQQAQNQQSQAQSQEQESQSAESESAVQSSQSSSGMSSQAQSQQQQAQSQEQESQSMSSRAQSQQSQAQSQEQESQSAQSGSDVEVSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSRGMSSQAQSQQQQSQLQQSQAQSQEQESQSAGSESAVQSSKSSQWNEQPSSKPTTTSSTPTVPSSEPRTGIPICSIWIRC

SEQ ID NO: 13 - DNA, 1077 bp, Hylaeus nubilosus
  source 1..1077: mol_type = other DNA
atgtcttcacgtgctcagtcacagcagtcccaggcccagagccaggagcaggaatcacagtccgctcagagcggttctgacgtggaggtgtccaagtccagctctggcaggtcatcccaggcccagtcacagcagcagcagtcccagctgcagcagagccaggctcagtctcaagaacaggaatcacagtccgccgagagcgaatctgacgtgcagtcctccaagtcatccaggggaatgtcctcccacgctcagagccagcagtctcaggcccagtctcaagagcaagagtcacagtccgctgagagcgaatctgccgtccagtcatccaagtcctcccgcggtatgtcatcccaggctcagagccagcagcagcagtctcagctgcagcagtcacaggctcagtctcaagagcaagaatctcagtccgcccagagcggatctgatgtcgaggtgtccaagtcctcctccggtcgctcctcccaggctcaatctcaacaacaacaatctcaattgcaacaatctcaggctcagtctcaggaacaagagtctcaatctgctgaatctgagtcagacgtgcagtcttcaaagtccagccgcggcatgtcttcacacgctcaatctcaacaatcccaggcccagtctcaggaacaagaatctcagtccgctcagagcggctctgacgtggaggtcagcaagtccagctctggacgttcatcccaagcccaatctcaacaacaacagtcacaattgcagcagtcacaggcccagtctcaggagcaagaatcacagtccgctggtagcgaatctgctgtccagtcctccaagtcatccagaggcatgtcctcccaggctcaatcccaacaacaacaatctcagttgcagcagagtcaagcccagtctcaagagcaggagtcacagtccgctggaagcgagtcagctgtgcagtcatccaagtcctcccagtggaacgaacagccttcatccaagcccaccactaccagctctactcctaccgtcccatcttcggagccacgcacgggcattcctatctgttctatttggattcgctgctaa

SEQ ID NO: 14 - protein, 358 aa, Hylaeus nubilosus
MSSRAQSQQSQAQSQEQESQSAQSGSDVEVSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSRGMSSQAQSQQQQSQLQQSQAQSQEQESQSAGSESAVQSSKSSQWNEQPSSKPTTTSSTPTVPSSEPRTGIPICSIWIRC

SEQ ID NO: 15 - DNA, 1191 bp, Hylaeus nubilosus
  source 1..1191: mol_type = other DNA
atgagcagccaggcccagagccagcagcagcagagccagctgcagcagagccaggcccagagccaggagcaggagagccagagcgccgagagcgagagcgacgtgcagagcagcaagagcagcagcggcaggagcagccaggcccagagccagcagagccaggcccagagccaggagcaggagagccagagcgcccagagcgagagcgacgtggaggtgagcaagagcagcagcggcaggagcagccaggcccagagccagcagcagcagagccagctgcagcagagccaggcccagagccaggagcaggagagccagagcgccgagagcgagagcgacgtgcagagcagcaagagcagcaggggcatgagcagccacgcccagagccagcagagccaggcccagagccaggagcaggagagccagagcgccgagagcgagagcgccgtgcagagcagcaagagcagcaggggcatgagcagccaggcccagagccagcagcagcagagccagctgcagcagagccaggcccagagccaggagcaggagagccagagcgcccagagcggcagcgacgtggaggtgagcaagagcagcagcggcaggagcagccaggcccagagccagcagcagcagagccagctgcagcagagccaggcccagagccaggagcaggagagccagagcgccgagagcgagagcgacgtgcagagcagcaagagcagcaggggcatgagcagcagggcccagagccagcagagccaggcccagagccaggagcaggagagccagagcgcccagagcggcagcgacgtggaggtgagcaagagcagcagcggcaggagcagccaggcccagagccagcagcagcagagccagctgcagcagagccaggcccagagccaggagcaggagagccagagcgcccagagcgagagcgacgtggaggtgagcaagagcagcagcggcaggagcagccaggcccagagccagcagcagcagagccagctgcagcagagccaggcccagagccaggagcaggagagccagagcgccggcagcgagagcgccgtgcagagcagcaagagcagccagtggaacgagcagcccagcagcaagcccaccaccaccatcagcgcccccaccgtgcccagcagcgagcccaggaccggcatccccgtgtgcaggatc

SEQ ID NO: 16 - protein, 397 aa, Hylaeus nubilosus
MSSQAQSQQQQSQLQQSQAQSQEQESQSAESESDVQSSKSSSGRSSQAQSQQSQAQSQEQESQSAQSESDVEVSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSRAQSQQSQAQSQEQESQSAQSGSDVEVSKSSSGRSSQAQSQQQQSQLQQSQAQSQEQESQSAQSESDVEVSKSSSGRSSQAQSQQQQSQLQQSQAQSQEQESQSAGSESAVQSSKSSQWNEQPSSKPTTTISAPTVPSSEPRTGIPVCRI

SEQ ID NO: 17 - DNA, 108 bp, Hylaeus nubilosus
  source 1..108: mol_type = other DNA
agcgagagcgccgtgcagagcagcaagagcagcagcggcatgagcagccaggcccagagccagcagcagcaggcccagagccaggagcaggagagccagagcgcccag

SEQ ID NO: 18 - protein, 36 aa, Hylaeus nubilosus
SESAVQSSKSSSGMSSQAQSQQQQAQSQEQESQSAQ

SEQ ID NO: 19 - DNA, 108 bp, Hylaeus nubilosus
  source 1..108: mol_type = other DNA
agcgagagcgacgtgcagagcagcaagagcagcaggggcatgagcagccacgcccagagccagcagagccaggcccagagccaggagcaggagagccagagcgcccag

SEQ ID NO: 20 - protein, 36 aa, Hylaeus nubilosus
SESDVQSSKSSRGMSSHAQSQQSQAQSQEQESQSAQ

SEQ ID NO: 21 - DNA, 69 bp, Hylaeus nubilosus
  source 1..69: mol_type = other DNA
atgagcagcagggcccagagccagcagagccaggcccagagccaggagcaggagagccagagcgcccag

SEQ ID NO: 22 - protein, 23 aa, Hylaeus nubilosus
MSSRAQSQQSQAQSQEQESQSAQ

SEQ ID NO: 23 - DNA, 33 bp, Hylaeus nubilosus
  source 1..33: mol_type = other DNA
agcgagagcgccgtgcagagcagcaagagcagc

SEQ ID NO: 24 - protein, 11 aa, Hylaeus nubilosus
SESAVQSSKSS

SEQ ID NO: 25 - DNA, 20870 bp, Hylaeus nubilosus
  source 1..20870: mol_type = other DNA
tactcattcagtgcaaagatgaaatgtgtcttgttattagttgtcatattcgcagtcgcggcttatgcccgacctcagtcgccacaccatggtcaccatggtgcgaagactactttgaaattccatccccaccatggattccgcaaacatcacggccatcctaatcatcataaaatggtaagtctttatttgagacatactttaaagtgtccaccatatatacattcaaaacggagctaaacgtatgctgcaaggcttatgtagcacgtatgtcaactatgatcttaaatgattttagtcttatacaatctatatttaccaataaaacatcatttcaactttcaaacgtaattgggtcgtagtcacaccgagtatacagtactcgacgctactttagcatcactgtccatcaagttgtcataaacttatcactatcacctaactgtgggcattatcaacaaatgttatgtgttcgatgatgaccatgcagctaaccagtaacaaaagcaagcagaaacaaagttttagattctacatcttgtcattataactgtacaattaaaactaattgattatatttataccgaacaaataatattaaacataatcatatttgaacaatttggaataagaacataagcaattatacataaaaacgattagataccacaattttcaatataacgaataactcctcatcataacaatcaatcggttatttcttgacaaataattatatttaacaaaccaatgtaaaaaataaagaagaaaatagattcataatctctacagtcgtgccattatacgttatatattaggtcacgatgctattcccccaaaaataatgaattatatccaagtcaaaaaaatacaaaattgaaaattaggatggacgcaaaagcattacactgctataacatctactacggtgggtttgcaggcttccaacagcgaaaaagaactagaatgggatgacaacgtacagcagaaatactggaatgagaaacgactccaatccgatagaaaacatgaatcagaattcgaacaatccaaatccgaaaacaaagtaacaaaatcgtccagtacacactctagccaacataatgagaaccatacggaactatctgagtcaaaatccacacatacggcacataaatcgtcaggaggaaaatcaagccaaatggaaagccaacaccaacaagctcaaaaccaacagtcccaagcccagagccaagaacaggaatcccagtctgcagaatctgaatccgctgttcagtcaagccaatcctccagtggaatgagcagccaagctcaaagccaacaacaacaagcccagagccaagaacaggaatcccagtcctgcagaatcagaatccgctgttcagtcaagccaatcctccagtggaatgagcagccaagctcaaagccaacaacaacaagcccagagccaagaacaggaatcccagtctgcagaatctgaatccgctgttcagtcaagcaaatcctccagtggaatgagcagccaagctcaaagccaacaacagcaagctcaagtccaacagtctcaagctcagagccaagaacaggagtcccagtctgcaggatctgaatccgccgttcagtcaagcaaatcctccagtggaatgagcagccaagctcaaagccaacagtcccaagctcagagccaagaacaggaatcccaatctgctcaatctggatccgatgttcaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggagtcacagtctgcaggatctgaatccgccgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagtagccaagctcaaagccaacaacaacaatctcagctccaacagtcccaagctcagagccaagaacaggaatcccagtctgcagaatctgaatccgccgttcagtcaagcaaatcctccagtggaatgagcagccaagctcaaagccaacaacaacaagctcagagccaagaacaggaatcgcaatctgctcaatctgaatccgccattcagtcaagcaaatcctccagtggaatgagcagccaagctcaaagccaacaacagcaagctcaagtccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaagctcaagtccaacagtctcaagctcagagccaagaacaggagtcccagtctgcaggatctgaatccgccgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagcagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggagtcccagtctgcaggatctgaatccgccgttcagtcaagcaaatcctccagtggaatgagcagccaagctcaaagccaacaacaacaagctcagagccaagaacaggaatcccaatctgctcaatctgaatccgccattcagtcaagcaaatcctccagtggaatgagtagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaagctcagagccaagaacaggaatcgcaatctgctcaatctgaatccgccattcagtcaagcaaatcctccagtggaatgagcagccaagctcaaagccaacaacagcaagctcaagtccaacagtcccaagctcaaagccaagaacaggaatcccagtctgcagaatctgaatccgatgttcagtcaagcaaatcctccaggggaatgagcagccatgctcaaagccaccagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaagctcaagtccaacagtctcaagctcagagccaagaacaggagtcccagtctgcaggatctgaatccgccgttcagtcaagcaaatcctccagtggaatgagcagccaagctcaaagccaacaatcaatcaagctcagagccaagaacaggaatcccaatctgctcaatctgaatccgccattcagtcaagcaaatcctccagtggaatgagcagccaagctcaaagccaacaacagcaagctcaagctccaacagtctcaagctcagagccaagaacaggaatcccaatctgctcaatctggatccgacgttcaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacagcaatctcaactgcaacagtcccaagctcagagccaagaacaggaatctcaatccgaccaggctgaatccgatgttcagtcaagcaaatcctccaggggaatgagcagccacgctcaaagccaacagtctcaagctcaaagccaggaacaggaatcccagtcagcagaatctgagtccgctgttcagtcaagcaaatcctccagtggaaggagcagccaagctcaaagccaacaacaacaatctcagctccaacagtcccaagctcagagccaagaacaggaatcccaatctgctcaatctgcatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtctagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacagcaatctcaactccaacagtcccaagctcagagccaagaacaggaatctcaatccgaccaggctgaatccgatgttcagtcaagcaaatcctccaggggaatgagcagccacgctcaaagccaacagtctcaagctcaaagccaggaacaggaatcccagtcagcagaatctgaatccgctgttcagtcaagcaaatcctccagtggaaggagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccaatctgctcaatctggatccgatgttgaagtcaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaagctcaactccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgacagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaatctcagctccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaatctcaactccaacagtcccaagctcaaagccaggaacaggaatctcaatccgaccaggctgaatcagatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgacgtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaatctcagctccaacagtcccaagctcagagccaagaacaggaatcccagtctgcaggatctgaatccgctgttcagtcaagcaaatcctctcagtggaatgagcagccaagctcaaagccaacaacaacaagctcaactccaacagtcccaagctcaaagccaagaacaggaatcccagtctgcagaatctgaatccgatgttcagtcaagcaaatcctccaggggaatgagcagccgtgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggaatgagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaagctcaactccaacagtcccaagctcagagccaagaacaggaatcccaatctgctcaatctggatccgatgttgacgtaagcaaatcctccagtggaaggagcagccaagctcaaagccaacaacaacaatctcagctccaacagtctcaagctcagagccaagaacaggaatctcaatccgaccaggctgaatccgatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccagtctgcagaatctggatccgctgttgcagtcaagcaaatcctccagtggaaggagcagccaagctcaaagccaacaacaacaatctcagctccaacagtcccaagctcagagccaagaacaggaatcccaatctgctcaatctggatccgatgttgaagtaagcaaatcctccaggggaatgagcagccatgctcaaagccaacagtctcaagctcaaagccaggaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcgtggcatgagcagccaagctcaaagccaacaacaacaatctcaactccaacagtcccaagctcaaagccaggaacaggaatctcaatccgaccaggctgaatcagatgttcagtcaagcaaatcctccaggggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccaatctgctcaatctgaatccgatgttcagtcaagcaaatcctctcagtggaatgagcagccaagctcaaagccaacaacaacaagctcaactccaacagtcccaagctcaaagccaagaacaggaatcccagtctgcagaatctgaatccgctgttcagtcaagcaaatcctccagtggaatgagcagccaagctcaaagccagcaacaacaatctcaactccaacagtcccaagctcaaagccaggaacaggaatcccagtcagcagaatctgagtccgctgttcagtcaagcaaatcctccagtggaatgagcagccaagctcaaagccaacaacagcaatcccaactgcaacagtcccaagctcagagccaagaacaggaatctcaatccgaccaggctgaatcagatgttcagtcaagcaaatcctccaggggaatgagcagccaagctcaaagccaacagtctcaagctcaaagccaggaacaggaatcccagtcagcagaatctgaatccgccgttcagtcaagcaaatcctccagtggaatgagcagccatgctcaaagccaacagtctcaagctcaaagccaggaacaggaatcccagtcagcagaatctgaatccgccgttcagtcaagcaaatcctctcgtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaatctcaactccaacagtcccaagctcaaagccaagaacaggaatcccagtctgcagaatctgaatccgatgttcagtcaagcaaatcctccaggggaatgagcagccatgctcaaagccaacagtcccaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctctcagtggaatgagcagccaagctcaaagccaacaacaacaagctcaactccaacagtcccaagctcagagccaagaacaggaatcccagtctgcagaatctgaatccgatgttcagtcaagcaaatcctccaggggaatgagcagccatgctcaaagccaacagtctcaagctcaaagccaggaacaggaatcccagtcagcagaatctgaatccgctgttcagtcaagcaaatcctccagtggaatgagcagccaagctcaaagccaacaacaacaatctcagctccaacagtctcaagctcagagccaagaacaggaatcccaatctgctcaatctggatccgatgttgaagtaagcaaatcctccagtggaaggagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaatctcagctccaacagtcccaagctcagagccaagaacaggaatcccagtctgcaggatctgaatccgccgttgagtcaagcaaatcctctcgtggaatgagcagccatgctcaaagccaacaatctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcagaatctgaatccgccgttcagtcaagcaaatcctccagtggaatgagcagccaagctcaaagccaacaacaacaatctcagctccaacagtcccaagctcagagccaagaacaggaatcccagtctgcagaatctgaatccgctgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaatctcagctccaacagtcccaagctcagagccaagaacaggaatcgcagtctgcagaatctgaatccgccgttgagtcaagcaaatcctctcagtggaatgagcagccatgctcaaagccaacagtctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagtcaagctcaaagccaacagtctcaagctcaaagccaggaacaggaatcccaatctgctcaatctggatccgacgttcaagtaagcaaatcgtccagtggaatgagcagccaagctcaaagccaacaacagcaagctcaactccaacagtcccaagctcagagccaagaacaggaatcccagtctgcaggatctgaatccgccgttcagtcaagcaaatcctccagtggaatgagcagccaagctcaaagccaacaacaacaatctcagctccaacagtcccaagctcagagccaagaacaggaatcccagtctgcaggatctgaatccgccgttgagtcaagcaaatcctctcagtggaatgagcagccatgctcaaagccaacaatctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcagaatctgaatccgccgttcagtcaagcaaatcctccagtggaatgagcagccaagctcaaagccaacaacaacaatctcagctccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatcagccgttcagtcaagcaaatcctccagtggaatgagcagccaagctcaaagccaacaacaacaatctcagctccaacagtcccaagctcagagccaagaacaggaatcgcagtctgcagaatctgaatccgccgttgagtcaagcaaatcctctcgtggaatgagcagccatgctcaaagccaacaatctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatcagccgttcagtcaagcaaatcctccagtggaatgagcagtcaagctcaaagccaacagtctcaagctcaaagccaggaacaggaatcccaatctgctcaatctggatccgacgttcaagtaagcaaatcgtccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggaatcccaatctgctcaatctggatccgacgttcaagtaagcaaatcctccagtggtaggagcagccaagctcaaagccaacaacaacaatctcaagtccaacagtcccaagctcagagccaagaacaggaatcccagtctgcaggatctgaatcagccgttcagtcaagcaaatcctccagtggaatgagcagccaagctcaaagccaacaacaacaatctcagctccaacagtcccaagctcagagccaagaacaggaatcgcagtctgcagaatctgaatccgccgttgagtcaagcaaatcctctcgtggaatgagcagccatgctcaaagccaacagtctcaagctcaaagccaggaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatccgccgttcagtcaagcaaatcctccagtggaatgagcagccaagctcaaagccaacaacaacaagctcaactccaacagtcccaagctcagagccaagaacaggaatcccagtctgcaggatctgaatcagccgttcagtcaagcaaatcctccagtggaatgagcagtcaagctcaaagccaacagtctcaagctcaaagccaggaacaggaatcccaatctgctcaatctggatccgacgttcaagtcaagcaaatcctccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggaatcccaatctgctcaatctggatccgacgttcaagtcaagcaaatcctccagtggtaggagcagccaagctcaaagccaacaacaacaatctcaagtccaacagtcccaagctcagagccaagaacaggaatcccagtctgcaggatctgaatcagccgttcagtcaagcaaatcctccagtggaatgagcagccaagctcaaagccaacaacaacaagctcaactccaacagtcccaagctcagagccaagaacaggaatcccagtctgcaggatctgaatcagccgttcagtcaagcaaatcctccagtggaatgagcagtcaagctcaaagccaacagtctcaagctcaaagccaggaacaggaatctcaatctgctcaatctggatccgccgttcagtcaagtaaatcctccagtggaatgagcagccaagctcaaagccaacaacagcaagctcaagtccaacagtctcaagctcagagccaagaacaggaatcccaatctgctcaatctgaatccgccattcagtcaagcaaatcctccagtggaatgagcagccaagctcaaagccaacaacagcaagctcaagtccaacagtctcaagctcagagccaagaacaggaatcccaatctgctcaatctggatccgacgttcaagtaagcaaatcctccagtggtaggagcagccaagctcaaagccaacaacaacaatctcaagtccaacagtctcaagctcagagccaagaacaggaatcccaatctgccgaatctgaatcggctgtgcagtcaagcaaatcctccagtggaaggagcagccaggctcaaagccaacaacaacaagctcaactccaacagtctcaagctcagagtcaagaagaggaatctgaatctgctcagtctgaatccgaagttcatgtaagcaaatcgtcaggtggaaagagcagccaagtgcagagtgaagaacaagaatctgaagtcgaagaatcccaatctgaaaaacaagtagaagactccgaatctggtaaggccgtatctgttagtcatagtagcaaatctgcaagtaaaaagtgcagccattcttcacacaaacaccatggatctgagggtaaacattcgaaatccacacacaatgtacactcacattcaagccatgagagtagtcattccggacataaacatcacgagtctgaagttgaatctgaacaggaggaatcgttggtcggcgaacagatgaagtggaagcaactgccagcgttgaagcttaagttgaaccgaaaacgtttgtcccttaaccgaatcaaggtgcactaggaattcatgtgatgggtgaaaagaatgaatcctttttagatggatgacactacttttacactgaccggcttagatttaaggaacacgttagataattattatgaatttatataattcgtaatttgccacatttgcagtagatggtagtattagatagtagatgcaactgtgcaacgctttgtagtgaattaaaatagatgtatttacagcaatcactttttgtttttatttggttgatgcattgtgatgatctgtcctccttatactgtttgatattatacgagaagcaactttcgcatatcataatcgaggttcttctacacattatgttactatacagctactttataataacggttgaagcgataagagggaagacgaagttttccacgacaacatatctacctatggtgtcgtacagcaagcggtaccacaaaaaggaagtgtcaaacttttcctattccgtttattgttggacgctctattacgaatactgcaattctcctattagaagtttgagctgtatatcgttacgtatgcgacgatcgttgttctaacgcgtgaactttctgtggacatcggacgcgttatcgagccgacgttcgcattttacattttccctgtggctttgttcactcgggcgtcacttcgatccgattgtcgcgtctgaaaagcggcaacgatattttctctatttccatcgatgcgcaccgtctgtctggccggataattaaacggaacggactgacctccttccaatccccgagcaacatgg

SEQ ID NO: 26 - protein, 19 aa, Hylaeus nubilosus
SKSTHTAHKSSGGKSSQME

SEQ ID NO: 27 - protein, 16 aa, Hylaeus nubilosus
PTTTSSTPTVPSSEPR

SEQ ID NO: 28 - protein, 11 aa, Hylaeus nubilosus
TGIPICSIWIR

SEQ ID NO: 29 - protein, 11 aa, Hylaeus nubilosus
SSQWNEQPSSK

SEQ ID NO: 30 - protein, 8 aa, Hylaeus nubilosus
NLYFQGAK

SEQ ID NO: 31 - protein, 10 aa, Hylaeus nubilosus
KHHGHPNHHK

SEQ ID NO: 32 - protein, 8 aa, Hylaeus nubilosus
TLKFHPHH

SEQ ID NO: 33 - protein, 86 aa, Hylaeus nubilosus
SSSGMSSQAQSQQQQAQLQQSQDQRQEQESQSAGSESAVKSSKSSSGMSSQAQSQQQQAQLQQSQAQSQEEESESAQSESEVHVSK

SEQ ID NO: 34 - protein, 68 aa, Hylaeus nubilosus
SQEQESQSAGSESAVQSSKSSSGMSSQAQSQQQQSQLQQSQAQSQEQESQSAGSESAVESSKSSRGMS

SEQ ID NO: 35 - protein, 71 aa, Hylaeus nubilosus
SQLQQSQAQSQEQESQSAESESAVQSSKSSSGMSSQAQSQQQQAQLQQSQAQSQEEESESAQSESEVHVSK

SEQ ID NO: 36 - protein, 44 aa, Hylaeus nubilosus
KSSSGMSSQAQSQQQQSQLQQSQAQSQEQESQSAESESAVESSK

SEQ ID NO: 37 - protein, 25 aa, Hylaeus nubilosus
GMSSQAQSQQQQSQLQQSQAQSQEQ

SEQ ID NO: 38 - protein, 47 aa, Hylaeus nubilosus
ESQSAQSGMSSQAQSQQQQAQVQQSQAQRQEQESQSAGSESAVKSSK

SEQ ID NO: 39 - protein, 42 aa, Hylaeus nubilosus
SSVMISQSQSQQQQAQVQQSQAQSQEQESQSAQSGSDVQVSK

SEQ ID NO: 40 - protein, 58 aa, Hylaeus nubilosus
QAQVQQSQAQSQEQESQSAQSESAVQTSKSSSGMRSQSQSQQQQAKLQQSQAQSQEQE

SEQ ID NO: 41 - protein, 33 aa, Hylaeus nubilosus
GMSSHSQSQQSQAQSQEQESQSAGSESAVQSSK

SEQ ID NO: 42 - protein, 14 aa, Hylaeus nubilosus
SQQQQAQVQQSQAQ

SEQ ID NO: 43 - protein, 26 aa, Hylaeus nubilosus
IQQSQAQSQEQESQSAGSESAVQSSK

SEQ ID NO: 44 - protein, 53 aa, Hylaeus nubilosus
SSQAQSQQQQSQVQQSQAQRQEQESQSAGSESAVKSSKSSSGMSSQAQSQQQQ

SEQ ID NO: 45 - protein, 32 aa, Hylaeus nubilosus
SQQQSQLQQSQAQSQQQESQSDQAESDVQSSK

SEQ ID NO: 46 - protein, 39 aa, Hylaeus nubilosus
QSAGCESAVQSSHSSSGMSSLAQSQQQQSQLQQSQAQRK

SEQ ID NO: 47 - protein, 17 aa, Hylaeus nubilosus
MKCVLLLVVIFAVAAYA

SEQ ID NO: 48 - protein, 17 aa, Hylaeus nubilosus
MKCVLLLVVIFAVAAYA

SEQ ID NO: 49 - DNA, 1602 bp, Hylaeus nubilosus
  source 1..1602: mol_type = other DNA
atgcgccctcagtctccacatcacggccatcacggtgctaaaactacgctgaagtttcacccgcatcacggctttcgtaagcatcatggtcacccgaaccaccataaggccagcaacagcgagaaagaactggagtgggatgataacgttcagcagaaatattggaatgaaaagcgcctgcaaagcgaccgtaaacacgagagcgaattcgagcaatccaagagcgagaataaagtcaccaagagcagcagcacgcacagcagccaacacaacgaaaatcacactgagctgagcgagtcgaagtccacccataccgcgcataagtcgagcggtggcaagagcagccaaatggaaagccaacaccagcaagcacaaaatcagcaatcccaagcgcaaagccaagaacaagagagccaatccgcggaaagcgaatcggcggtgcagagctcacaatctagcagcggtatgtccagccaagctcaaagccaacagcaacaagcccaaagccaggaacaagagtcccagagcatgagcagccgtgcgcagtcccagcagtctcaagcacaatctcaggagcaggagtctcaaagcgcacagagcggctctgacgttgaggtgtccaagtccagctccggtcgctctagccaggcccagtctcagcagcagcaatctcaactgcaacaatctcaagctcagagccaagagcaagaatctcaatctgcagaaagcgagagcgatgttcaatccagcaaatctagccgtggcatgagctcgcatgcacagagccaacagagccaagcgcaatctcaagaacaggaatcccaaagcgccgagagcgaaagcgcagttcaaagctctaaaagctctcgcggcatgtcaagccaggctcagtcacaacagcagcaaagccagctgcaacagtctcaggctcaatcacaagagcaagaatcccagtccgcgcagtctggcagcgacgttgaagtgagcaaatcgagctctggtagatcttcacaagcgcagagccagcaacaacagtcccaattgcaacaatcccaggcacagtcgcaagagcaggaaagccagtccgcagagagcgagtctgacgtccagagcagcaaatcttctcgcggtatgagcagccacgcgcagtcgcagcagtcccaagcccagtcccaggaacaggagagccaaagcgctcagagcggtagcgatgtcgaggtgagcaaaagcagcagcggccgtagcagccaggcacaatcgcagcaacagcagagccaactgcaacaaagccaagcacaaagccaggaacaagagtcccagtctgctggttccgagagcgccgttcagagctccaaaagcagccgtggtatgagctctcaagcccaatcccaacaacagcagtcgcaactgcagcaatcgcaggcgcaatcccaggagcaagagtctcagagcgcgggtagcgagagcgcggtccagtctagcaaaagctcccagtggaatgaacagccgagcagcaagccgaccacgaccagcagcacccctaccgtcccgtccagcgaaccgcgtacgggcattccgatttgcagcatctggatccgctgt

SEQ ID NO: 50 - protein, 534 aa, Hylaeus nubilosus
MRPQSPHHGHHGAKTTLKFHPHHGFRKHHGHPNHHKASNSEKELEWDDNVQQKYWNEKRLQSDRKHESEFEQSKSENKVTKSSSTHSSQHNENHTELSESKSTHTAHKSSGGKSSQMESQHQQAQNQQSQAQSQEQESQSAESESAVQSSQSSSGMSSQAQSQQQQAQSQEQESQSMSSRAQSQQSQAQSQEQESQSAQSGSDVEVSKSSSGRSSQAQSQQQQSQLQQSQAQSQEQESQSAESESDVQSSKSSRGMSSHAQSQQSQAQSQEQESQSAESESAVQSSKSSRGMSSQAQSQQQQSQLQQSQAQSQEQESQSAQSGSDVEVSKSSSGRSSQAQSQQQQSQLQQSQAQSQEQESQSAESESDVQSSKSSRGMSSHAQSQQSQAQSQEQESQSAQSGSDVEVSKSSSGRSSQAQSQQQQSQLQQSQAQSQEQESQSAGSESAVQSSKSSRGMSSQAQSQQQQSQLQQSQAQSQEQESQSAGSESAVQSSKSSQWNEQPSSKPTTTSSTPTVPSSEPRTGIPICSIWIRC

SEQ ID NO: 51 - DNA, 1074 bp, Hylaeus nubilosus
  source 1..1074: mol_type = other DNA
atgagcagccgtgcgcaatcccagcaatcccaagcacagtcccaagaacaggaatcccagagcgcgcaatctggtagcgatgtcgaagtgagcaaatcctctagcggtcgcagcagccaagcccagtctcaacagcagcagtctcaactgcagcagtcgcaggcgcagtcacaagagcaagaatcccagtcggcggagagcgagagcgatgtgcagtccagcaagagcagccgcggcatgtctagccacgcgcaaagccagcaatctcaggcacaaagccaggagcaagaaagccagtctgcagaaagcgagagcgctgtccagagctctaagtccagccgtggcatgtcctctcaggcccagtcccaacaacaacaaagccagttgcagcaaagccaagcgcagagccaagaacaagaatcgcagagcgctcagagcggcagcgatgtggaagtttctaagagcagcagcggccgttctagccaggcacagagccagcagcaacaatctcagctgcagcaatcgcaagcccaaagccaagagcaggaatctcagagcgcagagagcgaaagcgacgtgcagagcagcaagagctcacgtggcatgagcagccacgcccaatcgcagcagagccaggctcagtcgcaagagcaagagtcgcaaagcgcgcagagcggtagcgacgtcgaagtttccaagtccagcagcggtcgtagcagccaggcccagagccaacaacagcagtcccaactgcaacagtcccaggcgcaatcgcaggaacaggaaagccagagcgcaggcagcgaaagcgctgttcaaagctccaaatctagccgtggtatgtccagccaagctcagtcacagcagcagcaatcacagctgcaacaatcccaggcacaatctcaggaacaagagagccaaagcgcgggttccgagagcgcggttcaatctagcaaaagctcgcagtggaacgaacagccgtccagcaaaccgaccactaccagcagcaccccaacggttccgagcagcgagccgagaacgggtattccgatctgtagcatttggatccgctgc

SEQ ID NO: 52 - protein, 358 aa, Hylaeus nubilosus; identical to SEQ ID NO: 14